(19) United States
(12) Patent Application Publication
(10) Pub. No.: US 2026/0360473 A1
(43) Pub. Date: Nov. 12, 2026

(54) A METHOD FOR PROVIDING A CANDIDATE BIOLOGICAL SEQUENCE AND RELATED ELECTRONIC DEVICE

(71) Applicant: Novozymes A/S, Bagsvaerd (DK)

(72) Inventors: Jean-Marie MOUILLON, SODRA SANDBY (SE); Peter Fischer HALLIN, Holte (DK); Pernille Hvid CHRISTENSEN, Roskilde (DK); Eik BRAENDSTRUP, Bagsvaerd (DK); Dennis PULTZ, Hilleroed (DK)

(73) Assignee: Novozymes A/S, Bagsvaerd (DK)

(21) Appl. No.: 19/139,645

(22) PCT Filed: Dec. 19, 2023
(86) PCT No.: PCT/EP2023/086760

§ 371 Date: Jun. 16, 2025

(30) Foreign Application Priority Data
Dec. 20, 2022 (EP) 22215075.7
Jul. 19, 2023 (EP) 23186457.0

(51) Int. Cl. G16B 40/30; G06N 3/0455; G06N 3/0475

(52) U.S. Cl. CPC G16B 40/30; G06N 3/0455; G06N 3/0475

(57) ABSTRACT

Disclosed is a method, performed by an electronic device, for providing a candidate biological sequence. The method comprises obtaining input data indicative of an input biological sequence. The method comprises determining the candidate biological sequence by applying a generative non-unidirectional model to the input data. The method comprises providing biological sequence data indicative of the candidate biological sequence.

The present disclosure pertains to the field of bioinformatics. The present disclosure relates to a method for providing a candidate biological sequence and related electronic device.

### REFERENCE TO A SEQUENCE LISTING

This application contains a Sequence Listing in computer readable form, which is incorporated herein by reference.

### BACKGROUND

Finding biological sequences is largely based on screening of libraries. However, the chances of finding satisfactory ‘partner’ biological sequences, using such strategies, are limited due to the combinatorial nature of the problem.

### SUMMARY

Accordingly, there is a need for an electronic device and a method for providing a candidate biological sequence, which mitigate, alleviate, or address the shortcomings existing and provides a candidate biological sequence for a host cell based on an input biological sequence. For example, the disclosed technique allows identifying sequence-to-sequence pairs for the host cell expressing a polypeptide of interest, e.g., pairs of sequences that are to be used in combination.

For example, given an input biological sequence, this disclosure allows providing ‘partner’ sequences with an increased possibility of being compatible, e.g.: a new signal peptide compatible with a given mature polypeptide of interest, and/or a codon sequence compatible with a given amino acid sequence of a polypeptide of interest.

Disclosed is a method, performed by an electronic device, for providing a candidate biological sequence. The method comprises obtaining input data indicative of an input biological sequence. The method comprises determining the candidate biological sequence by applying a generative non-unidirectional model to the input data. The method comprises providing biological sequence data indicative of the candidate biological sequence. The candidate biological sequence is increasing compatibility with a host cell.

Disclosed is an electronic device. The electronic device comprises a memory circuitry, a processor circuitry, and an interface. The electronic device is configured to perform any of the methods according to any of the disclosed methods.

Disclosed is a computer readable storage medium storing one or more programs, the one or more programs comprising instructions, which when executed by an electronic device cause the electronic device to perform any of the methods according to the disclosed methods.

Disclosed is a recombinant host cell comprising, extra-chromosomal and/or in its genome a first polynucleotide encoding a control sequence, and a second polynucleotide operably linked to the first polynucleotide encoding a polypeptide of interest, wherein the first or second polynucleotide is the candidate biological sequence obtained by the method disclosed herein. Disclosed is a method of producing a polypeptide of interest, comprising cultivating the recombinant host cell disclosed herein under conditions conducive for production of the polypeptide of interest, and optionally recovering the polypeptide of interest.

It is an advantage of the present disclosure that the disclosed electronic device and the disclosed method allow generating a candidate biological sequence that is predicted to achieve compatibility with the host cell to some extent. This may lead to an improved efficiency in testing candidate biological sequences. The disclosed technique can be applied to output multiple candidate biological sequences (instead of one). The candidate biological sequences determined can then be subjected to multiple sequence alignment and analyzed for the presence of positions where suggestions are repeatedly emphasized (e.g., ‘conserved’)—such positions are prime candidates for being significant for the sequence-to-sequence match. The disclosed technique provides the possibility to apply various types of machine learning techniques additionally or alternatively to multiple sequence alignments. The present disclosure allows analyzing input biological sequences and candidate biological sequences to extract compatibility rules using the generative model disclosed herein (such as pairs of mature polypeptide of interest sequence and its native signal-peptide sequence). The disclosed technique allows adapting the method based on experimental data. The disclosed technique allows to output one or more candidate biological sequences that can be experimentally validated and, subsequently, used to adapt the method in an iterative fashion.

Thus, in one embodiment the method is an iterative generative model. The iterative generative model has the ability to respond to experimental data. This response may be achieved, e.g., by setting up a “plurality” of generative models, where each member has its own ‘focus’. I.e., each member has a ‘focus’ on a certain subset of all possible sequences, e.g., all possible candidate biological sequences. This “plurality” of generative models is defined as part of the training process, and/or prior to the training process.

For example, using a trained model sequences are generated which sequences then are evaluated experimentally. For example, in the first round all generators are used. In another example, a random subset of the generators is used. Such experimental data gives hints as to which subset of these generators gives the most relevant output sequences, e.g., for the next round of iteration.

As an example, a set of signal peptides is generated in a first round (e.g., where all generators are applied or a random subset hereof). Then the signal peptides containing Proline are associated with a higher yield. In this case, sampling in the next iteration around signal peptides containing Proline is prioritized. Thus, in the next iteration, generators that had a special focus on proline-containing signal peptides are prioritized (i.e., containing at least 1 proline), see FIG. 15.

The generators, preferably during training, have been set up to have their individual focus on ‘proline content’. Alternatively, ‘proline content’ is part of the ‘focus’ of said generators.

‘A plurality of generators’ is defined by (in addition to the input sequences) conditioning the model on properties of the candidate biological sequences. In the context of the present invention, these properties are referred to as ‘predetermined criterion’, since these have to some extent to be prespecified. Thus, in one example, in addition to the noise ‘z’, the method utilizes two inputs to the model: one or more input biological sequence and one or more predetermined criterion.

In a preferred embodiment, during training the predetermined criterion corresponds to an actual property of the training output sequence.

In another embodiment, during training the predetermined criterion corresponds to a predicted and/or observed property. For example, the property is intrinsic to the output sequence and/or a property that cannot be evaluated in isolation. The reason for this is, for example, that the generative model needs to ‘learn’ how to interpret the meaning of the predetermined criterion. For instance, in the case of signal peptides, we might have a predetermined criterion that simply counts the number of Prolines in the output sequence. During training the model will then learn to interpret the predetermined criterion as a proline count, simply because it learns to associate this value with the actual training output sequence.

For example, during training, the model is trained to generate outputs that mimic the training output sequence, e.g., true training output sequence, whenever the model detects a match between an input sequence and a predetermined criterion. For example, during training, the predetermined criterion describes an actual property of the output sequence. Preferably, there is actual information in the predetermined criterion (and also in the input sequence) that the model learns to exploit in this ‘mimicking’ process. In one example, as soon as the model learns to exploit this information, the model has been ‘trained’. For instance, at some point the model will have learned that every time it receives as input a proline count of 1, this will correspond to situations where the model is also asked to mimic an output sequence that has exactly 1 proline. In other words, the model learns to assign meaning to the input.

Thus, in a preferred embodiment after training the model ‘knows’ how to generate new sequences with increased compatibility whenever it receives as input a new input biological sequence and a predetermined criterion. In one example, after training the model, the predetermined criterion is value that can be specified to control the process of generating new candidate biological sequences.

Thus, by applying a data analysis method to experimental data, values of a predetermined criterion associated with yield can be identified and be feed back to the model as mentioned above (e.g. see example with ‘prioritizing generators that had a special focus on proline-containing signal peptides’ above).

In a preferred example, the method generates sequences, which are subsequently validated experimentally. Afterwards, data analysis is applied to extract learnings from the experimental data. Accordingly, a subset of generators is prioritized and a new iteration is initiated.

Thus, in a preferred embodiment, the method comprises at least one iteration, wherein each iteration comprises (i) experimental validation of at least one biological sequence data indicative of the candidate biological sequence, and optionally (ii) prioritization of one or more generators of a plurality generators based on the experimental validation.

In one embodiment, the method comprises at least two iterations. In one embodiment, the method comprises at least three iterations, e.g., at least four iterations, at least five iterations, or at least six iterations.

In other examples, in addition to the above preferred example, there is the possibility of prioritizing a subset of generators by user defined values. For example, in situations where our learnings come from other sources (i.e., other than the iterative campaign itself). For example, an omics experiment from a Pilot scale or production scale fermentation reveals that signal peptide sequences comprising 5 lysines are associated with highly secreted proteins under certain cultivation conditions. As a result, the knowledge about prioritizing high lysine content in signal peptide sequences can be utilized to generate signal peptides with increased compatibility.

For example, as shown in FIG. 12, describes an individual generator having a certain ‘focus’. In FIG. 12 the generator, which generator preferably is identified by a certain value of a predetermined criterion, focuses on a certain subset of candidate biological sequences. Within this subset the generator actively ‘controls’ a subset of the nucleotides or amino acids of the subset of sequences. In some examples, some nucleotides or amino acids of the subset of sequences vary only due to ‘noise’ and their value does not depend on the conditions of the generator in question. On the other hand, other nucleotides or amino acids of the subset of sequences may depend on the conditions of the generator and, hence, variations at these positions would be ‘under the control’ of the generator.

Exemplary, as shown in the examples, the present invention can be used to create artificial signal peptide coding sequences and/or artificial signal peptides (candidate biological sequences) which outperform native signal peptides (FIG. 10). Further, the present invention can be utilized for codon-optimization of artificial and/or native signal peptides. Using the method of the invention, such codon-optimization results in an up to a 100-fold improvement of protein yield (FIGS. 7 to 9). Also, the method of the invention, e.g., by codon-optimization of signal peptides, can be used to regulate and fine-tune protein expression (FIGS. 7 and 8). Notably, biological duplicates confirm the validity of the method (FIG. 9).

### BRIEF DESCRIPTION OF THE DRAWINGS

The above and other features and advantages of the present disclosure will become readily apparent to those skilled in the art by the following detailed description of exemplary embodiments thereof with reference to the attached drawings, in which:

FIG. 1 is a diagram illustrating an example implementation according to this disclosure,

FIGS. 2A-C are diagrams illustrating schematically example implementations according to this disclosure,

FIGS. 3A-B are a flow-chart illustrating an exemplary method, performed by an electronic device, for providing a candidate biological sequence according to this disclosure,

FIG. 4 is a block diagram illustrating an exemplary electronic device according to this disclosure,

FIG. 5 is an illustration of example results according to this disclosure; and

FIG. 6 is an illustration of example results according to this disclosure.

FIG. 7 shows a comparison of protease activity of different codon variants for two artificial signal peptides.

FIG. 8 shows a comparison of protease activities for different artificial codon variants of wild-type signal peptides and artificial signal peptides.

FIG. 9 shows biological replicates for different artificial codon variants of an artificial signal peptide.

FIG. 10 shows protease activities for artificial signal peptide (SP) codon variants and aprL SP control strains.

FIG. 11 shows an example of a plurality of generators, each specified by a predetermined criterion.

FIG. 12 shows an individual generator among a plurality of generators and how this is associated with a subset of candidate biological sequences.

FIG. 13 shows an iterative aspect of the method.

FIG. 14 shows the benefits of a data-driven prioritization of experiments for screening.

FIG. 15 shows an exemplary method of how experimental learnings can adapt the method to determine a new round of candidate biological sequences.

### SEQUENCE OVERVIEW

SEQ ID NOs:1 to 247 are artificial DNA sequences encoding signal peptides.

SEQ ID NOs: 248 to 289 are amino acid sequences of signal peptides. \hat{ }

SEQ ID NO: 290 is a wild-type aprL control signal peptide (encoded by SEQ ID NO: 247).

SEQ ID NO: 292 is a protease amino acid sequence (encoded by SEQ ID NO: 291).

SEQ ID NO: 248 is a wild-type signal peptide, encoded by the artificial codon variants of SEQ ID NOs: 1-10.

SEQ ID NO: 249 is a wild-type signal peptide, encoded by the artificial codon variants of SEQ ID NOs: 11-15.

SEQ ID NO: 252 is a wild-type signal peptide, encoded by the artificial codon variants of SEQ ID NOs: 32-41.

SEQ ID NO: 253 is a wild-type signal peptide, encoded by the artificial codon variants of SEQ ID NOs: 42-52.

SEQ ID NO: 255 is a wild-type signal peptide, encoded by the artificial codon variants of SEQ ID NOs: 60-67.

SEQ ID NO: 256 is a wild-type signal peptide, encoded by the artificial codon variants of SEQ ID NOs: 68-74.

SEQ ID NO: 257 is a wild-type signal peptide, encoded by the artificial codon variants of SEQ ID NOs: 75-84.

SEQ ID NO: 258 is a wild-type signal peptide, encoded by the artificial codon variants of SEQ ID NOs: 85-98.

SEQ ID NO: 264 is a wild-type signal peptide, encoded by the artificial codon variants of SEQ ID NOs: 116-129.

SEQ ID NO: 265 is a wild-type signal peptide, encoded by the artificial codon variants of SEQ ID NOs: 130-137.

SEQ ID NO: 266 is a wild-type signal peptide, encoded by the artificial codon variants of SEQ ID NOs: 138-147.

SEQ ID NO: 269 is a wild-type signal peptide, encoded by the artificial codon variants of SEQ ID NOs: 162-167.

SEQ ID NO: 274 is a wild-type signal peptide, encoded by the artificial codon variants of SEQ ID NOs: 184-193.

SEQ ID NO: 276 is a wild-type signal peptide, encoded by the artificial codon variants of SEQ ID NOs: 196-209.

SEQ ID NO: 278 is a wild-type signal peptide, encoded by the artificial codon variants of SEQ ID NOs: 217-221.

SEQ ID NO: 279 and SEQ ID NO: 282 is an artificial signal peptide, encoded by the artificial codon variants of SEQ ID NOs: 222-228 and 237.

SEQ ID NO: 280 is an artificial signal peptide, encoded by the artificial codon variants of SEQ ID NOs: 229-235.

### DETAILED DESCRIPTION

Various exemplary embodiments and details are described hereinafter, with reference to the figures when relevant. It should be noted that the figures may or may not be drawn to scale and that elements of similar structures or functions are represented by like reference numerals throughout the figures. It should also be noted that the figures are only intended to facilitate the description of the embodiments. They are not intended as an exhaustive description of the disclosure or as a limitation on the scope of the disclosure. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

The figures are schematic and simplified for clarity, and they merely show details which aid understanding the disclosure, while other details have been left out. Throughout, the same reference numerals are used for identical or corresponding parts.

Unless defined otherwise or clearly indicated by context, all technical and scientific terms used herein have the same meaning as commonly understood by one of ordinary skill in the art to which this invention belongs.

Amylase: The term “amylase” means a polypeptide having amylase activity, such as an alpha-amylase (EC 3.2.1.1) that catalyzes the hydrolyzation of the 1,4-α-glucosidic linkages in amylose and amylopectin. Suitable amylases may be an alpha-amylase or a glucoamylase and may be of bacterial or fungal origin.

Cutinase: In one example the input sequence is a polynucleotide sequence encoding a cutinase, and/or an amino acid sequence comprising a cutinase. The term “cutinase” means a polypeptide having cutinase activity (EC 3.1.1.74), such as polyethylene terephthalate (PET) hydrolase activity, that catalyzes the hydrolysis of cutin and/or the hydrolysis of p-nitrophenyl esters of hexadecenoic acid.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon, such as ATG, GTG, or TTG, and ends with a stop codon, such as TAA, TAG, or TGA. The coding sequence may be a genomic DNA, cDNA, synthetic DNA, or a combination thereof.

Control sequences: The term “control sequences” means nucleic acid sequences and/or polypeptide sequences involved in regulation of expression of a polynucleotide in a specific organism or in vitro. Each control sequence may be native (i.e., from the same gene) or heterologous (i.e., from a different gene) to the polynucleotide encoding the polypeptide, and native or heterologous to each other. Such control sequences include, but are not limited to leader, polyadenylation, prepropeptide, propeptide, signal peptide, promoter, terminator, enhancer, and transcription or translation initiator and terminator sequences. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide. In one embodiment the control sequence is a nucleic acid sequence encoding a signal peptide.

Expression: The term “expression” means any step involved in the production of a polypeptide including, but not limited to, transcription, post-transcriptional modification, translation, post-translational modification, and secretion.

Compatibility: The term “compatibility” means improving, modifying and/or increasing one or more expression steps for a polypeptide of interest selected from a list including transcription, post-transcriptional modification, translation, post-translational modification, secretion, phenotypic trait, and yield.

Embedding: “Embedding” means an abstract numerical representation indicative of a biological object such as a biological sequence (or a protein structure). For example, an embedding is the result of applying a machine learning model to a large data set with the intent of doing transfer learning.

Expression vector: An “expression vector” refers to a linear or circular DNA construct comprising a DNA sequence encoding a polypeptide, which coding sequence is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

Extension: The term “extension” means an addition of one or more amino acids to the amino and/or carboxyl terminus of a polypeptide, or an addition of one or more nucleic acids to the 5′ and/or 3′ terminus of the nucleic acid sequence. Preferably, the “extended” polypeptide remains its activity and/or specificity.

Fragment: The term “fragment”, when referring to a protein, means a polypeptide, a catalytic domain, or a binding module having one or more amino acids absent from the amino and/or carboxyl terminus of the mature polypeptide, catalytic domain, or binding module. The term “fragment”, when referring to a nucleic acid sequence, means a nucleic acid sequence having one or more nucleic acids absent from the 5′ and/or 3′ terminus of the nucleic acid sequence. Preferably, the “fragmented” polypeptide remains its activity and/or specificity.

Fusion polypeptide: The term “fusion polypeptide” is a polypeptide in which one polypeptide is fused at the N-terminus and/or the C-terminus of a polypeptide of the present invention. A fusion polypeptide is produced by fusing a polynucleotide encoding another polypeptide to a polynucleotide of the present invention, or by fusing two or more polynucleotides of the present invention together. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779). A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

Heterologous: The term “heterologous” means, with respect to a host cell, that a polypeptide or nucleic acid does not naturally occur in the host cell. The term “heterologous” means, with respect to a polypeptide or nucleic acid, that a control sequence, e.g., promoter, of a polypeptide or nucleic acid is not naturally associated with the polypeptide or nucleic acid, i.e., the control sequence is from a gene other than the gene encoding the mature polypeptide.

Host Strain or Host Cell: A “host strain” or “host cell” is an organism into which an expression vector, phage, virus, or other DNA construct, including a polynucleotide encoding a polypeptide of the present invention has been introduced. Exemplary host strains are microorganism cells (e.g., bacteria, filamentous fungi, and yeast) capable of expressing the polypeptide of interest and/or fermenting saccharides. The term “host cell” includes protoplasts created from cells.

Introduced: The term “introduced” in the context of inserting a nucleic acid sequence into a cell, means “transfection”, “transformation” or “transduction,” as known in the art.

Mature polypeptide: The term “mature polypeptide” means a polypeptide in its mature form following N-terminal and/or C-terminal processing (e.g., removal of signal peptide). In one aspect, the mature polypeptide is an amylase. Suitable amylases include amylases having SEQ ID NO: 3 in WO 95/10603 or variants having 90% sequence identity to SEQ ID NO: 3. Preferred variants are described in WO 94/02597, WO 94/18314, WO 97/43424 and SEQ ID NO: 4 of WO 99/019467.

In one aspect the mature polypeptide is a protease.

Mature polypeptide coding sequence: The term “mature polypeptide coding sequence” means a polynucleotide that encodes a mature polypeptide. In one aspect, the mature polypeptide coding sequence encodes an amylase having SEQ ID NO: 3 in WO 95/10603 or variants having 90% sequence identity to SEQ ID NO: 3. In one aspect the mature polypeptide coding sequence encodes a protease.

Native: The term “native” means a nucleic acid or polypeptide naturally occurring in a host cell.

Nucleic acid: The term “nucleic acid” encompasses DNA, RNA, heteroduplexes, and synthetic molecules capable of encoding a polypeptide. Nucleic acids may be single stranded or double stranded, and may be chemical modifications. The terms “nucleic acid” and “polynucleotide” are used interchangeably. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5′-to-3′ orientation.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature, or which is synthetic, and which comprises one or more control sequences operably linked to the nucleic acid sequence.

Operably linked: The term “operably linked” means that specified components are in a relationship (including but not limited to juxtaposition) permitting them to function in an intended manner. For example, a regulatory sequence is operably linked to a coding sequence such that expression of the coding sequence is under control of the regulatory sequence.

Protease: In one aspect preferred polypeptides of interest and/or input biological sequences include a protease. Suitable proteases include those of bacterial, fungal, plant, viral or animal origin e.g. microbial or vegetable origin. Microbial origin is preferred. Chemically modified or protein engineered variants are included. It may be an alkaline protease, such as a serine protease or a metalloprotease. A serine protease may for example be of the S1 family, such as trypsin, or the S8 family such as subtilisin. A metalloproteases protease may for example be a thermolysin from e.g. family M4 or other metalloprotease such as those from M5, M7 or M8 families. A non-limiting example of a protease is shown in SEQ ID NO: 292.

The term “subtilases” refers to a sub-group of serine protease according to Siezen et al., Protein Engng. 4 (1991) 719-737 and Siezen et al. Protein Science 6 (1997) 501-523. Serine proteases are a subgroup of proteases characterized by having a serine in the active site, which forms a covalent adduct with the substrate. The subtilases may be divided into 6 sub-divisions, i.e. the Subtilisin family, the Thermitase family, the Proteinase K family, the Lantibiotic peptidase family, the Kexin family and the Pyrolysin family.

Examples of subtilases are those derived from Bacillus such as Bacillus lentus, B. alkalophilus, B. subtilis, B. amyloliquefaciens, Bacillus pumilus and Bacillus gibsonii described in; U.S. Pat. No. 7,262,042 and WO09/021867, and subtilisin lentus, subtilisin Novo, subtilisin Carlsberg, Bacillus licheniformis, subtilisin BPN′, subtilisin 309, subtilisin 147 and subtilisin 168 described in WO89/06279 and protease PD138 described in (WO93/18140). Other useful proteases may be those described in WO92/175177, WO01/016285, WO02/026024 and WO02/016547. Examples of trypsin-like proteases are trypsin (e.g. of porcine or bovine origin) and the Fusarium protease described in WO89/06270, WO94/25583 and WO05/040372, and the chymotrypsin proteases derived from Cellulomonas described in WO05/052161 and WO05/052146.

A further preferred protease is the alkaline protease from Bacillus lentus DSM 5483, as described for example in WO95/23221, and variants thereof which are described in WO92/21760, WO95/23221, EP1921147 and EP1921148.

Examples of metalloproteases are the neutral metalloprotease as described in WO07/044993 (Genencor Int.) such as those derived from Bacillus amyloliquefaciens.

Suitable commercially available protease enzymes include those sold under the trade names Alcalase®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Neutrase®, Everlase® and Esperase® (Novozymes A/S), those sold under the tradename Maxatase®, Maxacal®, Maxapem®, Purafect®, Purafect Prime®, Preferenz™ Purafect MA®, Purafect Ox®, Purafect OxP®, Puramax®, Properase®, Effectenz™, FN2®, FN3®, FN4®, Excellase®, Opticlean® and Optimase® (Danisco/DuPont), Axapem™ (Gist-Brocases N.V.), BLAP (sequence shown in FIG. 29 of U.S. Pat. No. 5,352,604) and variants hereof (Henkel AG) and KAP (Bacillus alkalophilus subtilisin) from Kao.

Purified: The term “purified” means a nucleic acid, polypeptide or cell that is substantially free from other components as determined by analytical techniques well known in the art (e.g., a purified polypeptide or nucleic acid may form a discrete band in an electrophoretic gel, chromatographic eluate, and/or a media subjected to density gradient centrifugation). A purified nucleic acid or polypeptide is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight or on a molar basis). In a related sense, a composition is enriched for a molecule when there is a substantial increase in the concentration of the molecule after application of a purification or enrichment technique. The term “enriched” refers to a compound, polypeptide, cell, nucleic acid, amino acid, or other specified material or component that is present in a composition at a relative or absolute concentration that is higher than a starting composition.

In one aspect, the term “purified” as used herein refers to the polypeptide or cell being essentially free from components (especially insoluble components) from the production organism. In other aspects. the term “purified” refers to the polypeptide being essentially free of insoluble components (especially insoluble components) from the native organism from which it is obtained. In one aspect, the polypeptide is separated from some of the soluble components of the organism and culture medium from which it is recovered. The polypeptide may be purified (i.e., separated) by one or more of the unit operations filtration, precipitation, or chromatography.

Accordingly, the polypeptide may be purified such that only minor amounts of other proteins, in particular, other polypeptides, are present. The term “purified” as used herein may refer to removal of other components, particularly other proteins and most particularly other enzymes present in the cell of origin of the polypeptide. The polypeptide may be “substantially pure”, i.e., free from other components from the organism in which it is produced, e.g., a host organism for recombinantly produced polypeptide. In one aspect, the polypeptide is at least 40% pure by weight of the total polypeptide material present in the preparation. In one aspect, the polypeptide is at least 50%, 60%, 70%, 80% or 90% pure by weight of the total polypeptide material present in the preparation. As used herein. a “substantially pure polypeptide” may denote a polypeptide preparation that contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polypeptide material with which the polypeptide is natively or recombinantly associated.

It is, therefore, preferred that the substantially pure polypeptide is at least 92% pure, preferably at least 94% pure, more preferably at least 95% pure, more preferably at least 96% pure, more preferably at least 97% pure, more preferably at least 98% pure, even more preferably at least 99% pure, most preferably at least 99.5% pure by weight of the total polypeptide material present in the preparation. The polypeptide of the present invention is preferably in a substantially pure form (i.e., the preparation is essentially free of other polypeptide material with which it is natively or recombinantly associated). This can be accomplished, for example by preparing the polypeptide by well-known recombinant methods or by classical purification methods.

Recombinant: The term “recombinant” is used in its conventional meaning to refer to the manipulation, e.g., cutting and rejoining, of nucleic acid sequences to form constellations different from those found in nature. The term recombinant refers to a cell, nucleic acid, polypeptide or vector that has been modified from its native state. Thus, for example, recombinant cells express genes that are not found within the native (non-recombinant) form of the cell, or express native genes at different levels or under different conditions than found in nature. The term “recombinant” is synonymous with “genetically modified” and “transgenic”.

Recover: The terms “recover” or “recovery” means the removal of a polypeptide from at least one fermentation broth component selected from the list of a cell, a nucleic acid, or other specified material, e.g., recovery of the polypeptide from the whole fermentation broth, or from the cell-free fermentation broth, by polypeptide crystal harvest, by filtration, e.g., depth filtration (by use of filter aids or packed filter medias, cloth filtration in chamber filters, rotary-drum filtration, drum filtration, rotary vacuum-drum filters, candle filters, horizontal leaf filters or similar, using sheed or pad filtration in framed or modular setups) or membrane filtration (using sheet filtration, module filtration, candle filtration, microfiltration, ultrafiltration in either cross flow, dynamic cross flow or dead end operation), or by centrifugation (using decanter centrifuges, disc stack centrifuges, hydro cyclones or similar), or by precipitating the polypeptide and using relevant solid-liquid separation methods to harvest the polypeptide from the broth media by use of classification separation by particle sizes. Recovery encompasses isolation and/or purification of the polypeptide.

Sequence identity: The relatedness between two amino acid sequences or between two nucleotide sequences is described by the parameter “sequence identity”.

For purposes of the present invention, the sequence identity between two amino acid sequences is determined as the output of “longest identity” using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the -nobrief option must be specified in the command line. The output of Needle labeled “longest identity” is calculated as follows:

(Identical Residues×100)/(Length of Alignment−Total Number of Gaps in Alignment)

For purposes of the present invention, the sequence identity between two polynucleotide sequences is determined as the output of “longest identity” using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 6.6.0 or later. The parameters used are a gap open penalty of 10, a gap extension penalty of 0.5, and the EDNAFULL (EMBOSS version of NCBI NUC4.4) substitution matrix. In order for the Needle program to report the longest identity, the nobrief option must be specified in the command line. The output of Needle labeled “longest identity” is calculated as follows:

(Identical Deoxyribonucleotides×100)/(Length of Alignment−Total Number of Gaps in Alignment)

Signal Peptide: A “signal peptide” is a sequence of amino acids attached to the N-terminal portion of a protein, which facilitates the secretion of the protein outside the cell. The mature form of an extracellular protein lacks the signal peptide, which is cleaved off during the secretion process. A non-limiting example for a well-known signal peptide is the aprL signal peptide shown in SEQ ID NO: 290.

Subsequence: The term “subsequence” means a polynucleotide having one or more nucleotides absent from the 5′ and/or 3′ end of a mature polypeptide coding sequence.

Variant: The term “variant” means a polypeptide a man-made mutation, i.e., a substitution, insertion (including extension), and/or deletion (e.g., truncation), at one or more positions. A substitution means replacement of the amino acid occupying a position with a different amino acid; a deletion means removal of the amino acid occupying a position; and an insertion means adding 1-5 amino acids (e.g., 1-3 amino acids, in particular, 1 amino acid) adjacent to and immediately following the amino acid occupying a position.

Wild-type: The term “wild-type” in reference to an amino acid sequence or nucleic acid sequence means that the amino acid sequence or nucleic acid sequence is a native or naturally-occurring sequence. As used herein, the term “naturally-occurring” refers to anything (e.g., proteins, amino acids, or nucleic acid sequences) that is found in nature. Conversely, the term “non-naturally occurring” refers to anything that is not found in nature (e.g., recombinant nucleic acids and protein sequences produced in the laboratory or modification of the wild-type sequence).

The chances of finding satisfactory ‘partner’ biological sequences, using screening strategies, are limited due to the combinatorial nature of the problem. For example, finding the best 10-mer peptide (e.g., a peptide consisting of 10 amino acids) that is to be used in combination with some biological sequence of interest requires screening 20\hat{ }10 different biological sequences (if only the 20 different amino acids from the standard genetic code are considered). This illustrates that finding satisfactory sequences by screening clearly is a tedious task and possibly an unfeasible task in a reasonable time frame, especially as biological sequences most often include more than 10 amino acids.

The present disclosure allows extracting learnings from native biological sequence pairs, e.g. as the biological sequences occur in nature. For example, the biological sequence pairs can be found in a database, such as a public database and/or a private database (such as National Center for Biotechnology Information NCBI database and/or a Nucleotide Archive e.g. EMBL). It may be envisaged to transfer the extracted learning to the experimental settings. The present disclosure allows learning compatibility rules from native sequence pairs provided by a database and providing the learned compatibility rules. It may be envisaged that the compatibility rules are further adapted to experimental settings.

The present disclosure allows some interaction between machine learning approaches and experimental approaches, e.g. using an iterative generative model as described above, and/or at least one iteration as described above. Machine learning-based analysis of biological sequences and experimental screening approaches contribute with two different layers of learnings. For example, the first layer of learnings allows extraction of complex biological rules that need to be obeyed while the second layer of learning accumulates data specific to the experimental settings.

In the disclosed technique, for example, the learning extracted is used to provide (using a Deep Learning approach, such as a generative model) a relevant subset of candidate biological sequences that can now be feasibly screened using experimental methods.

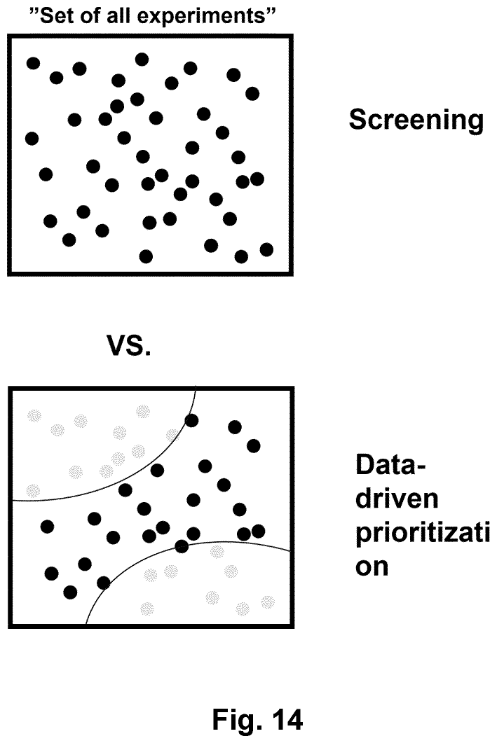

By applying the generative model, the disclosed technique may lead to unlocking the potential of experimental screening approaches by markedly reducing the complexity of the process of finding satisfactory ‘partner’ biological sequences. An example for such approach is shown in FIG. 14, where the model prioritizes according to experimental data, e.g., by doing one or more iteration. In other words, the actual quality of a candidate biological sequence is validated by experiments. In some examples, the learnings can be used as feedback to update the generative model (thus ‘informing’ the generative model about the quality of the suggestions).

The present disclosure provides a method, performed by an electronic device, for providing a candidate biological sequence. In other words, the method can be a computer-implemented method.

The method comprises obtaining input data indicative of an input biological sequence. The input data can be associated with the input biological sequence and/or be representative of the input biological sequence. The input data comprises data representative of the input biological sequence, such as data representative of one or more properties of the input biological sequence. In some examples, the one or more properties of the input data include one or more of: a sequence of amino acids, a sequence of nucleic acids, a three-dimensional structure of the input biological polypeptide sequence (e.g. obtained by Alpha-Fold2), a folding of the input biological sequence, and a pairing of nucleic acids.

In one embodiment the input biological sequence is the amino acid sequence shown in SEQ ID NO: 292.

In one embodiment the input biological sequence is the polynucleotide sequence shown in SEQ ID NO: 291.

In one embodiment the input biological sequence is an amino acid sequence selected from the list of SEQ ID NOs: 248 to 290.

In one embodiment the input biological sequence is a polynucleotide sequence selected from the list of SEQ ID NOs: 1 to 247.

In some examples, the method comprises obtaining host cell data associated with a host cell of interest. For example, the host cell data can indicate that the host cell is any of the host cells disclosed herein, such as a Bacillus host cell.

In some examples, the method comprises obtaining information indicating the type of biological sequence to be determined as candidate biological sequence. For example, a user can provide information indicating that the candidate biological sequences to be determined are one or more of: a control sequence, a nucleic acid sequence encoding a control sequence, an amino acid sequence of a polypeptide of interest, and a nucleic acid sequence encoding a polypeptide of interest.

The method comprises determining the candidate biological sequence by applying a generative model to the input data. For example, the candidate biological sequence is determined for compatibility with a host cell, e.g., targeting compatibility with a given host cell, and/or for increasing compatibility with the given host cell. In other words, for example, the generative model applied to the input data aims at increasing one or more expression steps for a polypeptide of interest in a host cell, e.g. increasing or modifying one or more of: transcription, post-transcriptional modification, translation, post-translational modification, folding, secretion, phenotypic trait, and yield for a polypeptide of interest in a host cell. Yield may be intra-cellular and/or extra-cellular. In other words, yield may be seen as a target performance parameter to optimise when determining the candidate biological sequence. It may be noted that yield may be optimized via various steps, such as modified secretion, modified transcription, modified translation, separately or jointly.

In some examples, the generative model generates, based on the input data, the candidate biological sequence. In some examples, the candidate biological sequence may be determined based on one or more of: host cell data, input data, and information indicating the type of biological sequence to be determined as candidate biological sequence.

The method comprises providing biological sequence data indicative of the candidate biological sequence. In some examples, the biological sequence data is associated with and/or representative of the candidate biological sequence. In some examples, the biological sequence data indicative of the candidate biological sequence is provided to a user device for experiments and/or for production. In some examples, the biological sequence data is associated with and/or representative of the candidate biological sequence. The biological sequence data comprises data representative of the candidate biological sequence, such as data representative of one or more properties of the candidate biological sequence. In some examples, the one or more properties of the candidate biological sequence include one or more of: a sequence of amino acids, a sequence of nucleic acids, a three-dimensional structure of the input biological polypeptide sequence, a folding of the input biological sequence, and a pairing of nucleic acids.

In one or more preferred example methods, the input biological sequence is associated with a donor organism that is not related to the host cell. For example, for a Bacillus host cell, the input biological sequence is derived from a donor organism that is not Bacillus. In one example the input biological sequence is derived from Alkalihalobacillus clausii.

In some examples, the input biological sequence may be derived from metagenomics.

In one or more example methods, the candidate biological sequence is a non-native biological sequence, for example a sequence that has not been referenced.

In some examples, the generative model is a generative non-unidirectional model. A generative non-unidirectional model may be seen as model that maps the input data to one or more candidate biological sequences, e.g. in one go. In other words, in some examples, the candidate biological sequence is not generated unidirectionally. For example, a generative unidirectional model generates a sequence of four nucleotides in the following sequential manner, nucleotide by nucleotide, e.g.: ‘G’, and then, ‘GC’ and then, ‘GCA’ and then, ‘GCAC’. In other words, for example, in a generative unidirectional model, the first nucleotide is outputted independently of the nucleotide coming next and only the later nucleotides are allowed to be dependent on the earlier nucleotides in the sequence.

In some examples, the generative non-unidirectional model generates sequence of four nucleotides all at once, e.g., without taking into account previous and subsequent nucleotides, e.g.: ‘GCAC’. In some examples, the model generates sequences of four nucleotides all at once, with taking into account previous and subsequent nucleotides. In some examples, the generative non-unidirectional model determines the entire sequence all at once and therefore does not need to rely on the dependencies between the first nucleotide and the subsequent nucleotides. In one particular embodiment, the generative non-unidirectional model does not need to learn the interdependence between the positions of a nucleotide in a sequence.

In one or more example methods, the input biological sequence is one or more of: an amino acid sequence of a polypeptide of interest, a nucleic acid sequence encoding a polypeptide of interest, a control sequence, and a nucleic acid sequence encoding a control sequence. In some examples, the polypeptide of interest is the amylase polypeptide shown in SEQ ID NO: 3 in WO 95/10603 or variants having 90% sequence identity to SEQ ID NO: 3. In some examples, the nucleic acid sequence encoding a polypeptide of interest is the nucleic acid sequence encoding the amylase of SEQ ID NO: 3 in WO 95/10603. In some examples, the control sequence is the signal peptide sequence of Bacillus stearothermophilus alpha-amylase. In some examples, the nucleic acid sequence encoding control sequence is nucleic acid sequence encoding the signal peptide of Bacillus stearothermophilus alpha-amylase.

In some examples, when the input biological sequence is a nucleic acid sequence, the 5′ end portion of the biological encoding the polypeptide of interest, preferably positions 1-75 of the input biological sequence (such as positions 1-60, such as positions 1-50, such as positions 1-35 etc.) can replace the input biological sequence of a full gene encoding a full polypeptide of interest.

In some examples, when the input biological sequence is an amino acid sequence, the N-terminal portion of the sequence of the polypeptide of interest, preferably positions 1-25 of the input biological sequence (such as positions 1-20, such as positions 1-15, such as positions 1-10, such as positions 1-7, etc.) can replace the biological input sequence of a full sequence of the polypeptide of interest or of a part of a sequence of the polypeptide of interest.

In one or more example methods, the candidate biological sequence is one or more of: a control sequence, e.g., an expression control sequence, a nucleic acid sequence encoding a control sequence, e.g., an expression control sequence, an amino acid sequence of a polypeptide of interest, and a nucleic acid sequence encoding a polypeptide of interest.

In one or more example methods, the input biological sequence is an amino acid sequence of a polypeptide of interest and/or a nucleic acid sequence encoding a polypeptide of interest. In one or more example methods, the candidate biological sequence is a nucleic acid sequence increasing compatibility with a host cell. Compatibility may be based on the context of the experiment with the host cell. Compatibility may be characterized by one or more compatibility rules which may be learned by the generative model disclosed herein. In some examples, the candidate biological sequence is determined such that the candidate biological sequence increases and/or modifies one or more of: transcription, post-transcriptional modification, translation, post-translational modification, folding, secretion, phenotypic trait, and yield for a polypeptide of interest in the host cell.

In one or more example methods, the input biological sequence is an amino acid sequence of a polypeptide of interest and/or a nucleic acid sequence encoding a polypeptide of interest. In one or more example methods, the candidate biological sequence is a control sequence and/or a nucleic acid sequence encoding a control sequence. In some examples, the input biological sequence is an amino acid sequence of a polypeptide of interest and/or a nucleic acid sequence encoding a polypeptide of interest and the candidate biological sequence determined is a control sequence and/or a nucleic acid sequence encoding a control sequence. Stated differently, in some examples where the input data is indicative of an amino acid sequence of a polypeptide of interest and/or a nucleic acid sequence encoding a polypeptide of interest, the candidate biological sequence which is determined by applying the generative model to the input data is a control sequence and/or a nucleic acid sequence encoding a control sequence.

In one or more example methods, the input biological sequence is a control sequence and/or a nucleic acid sequence encoding a control sequence. In one or more example methods, the candidate biological sequence is a nucleic acid sequence encoding a polypeptide of interest. In some examples, the input biological sequence is a control sequence and/or a nucleic acid sequence encoding a control sequence, and the candidate biological sequence determined is a nucleic acid sequence encoding a polypeptide of interest (such as a codon sequence). Stated differently, in some examples where the input data is indicative of a control sequence and/or a nucleic acid sequence encoding a control sequence, the candidate biological sequence which is determined by applying the generative model to the input data is a nucleic acid sequence encoding a polypeptide of interest.

In some examples, where the input data is indicative of an amino acid sequence of a polypeptide of interest, and a first candidate biological sequence determined is a control sequence, the method applies the generative model to the first candidate biological sequence determined as a control sequence (such as a signal peptide) for generating a second candidate biological sequence being a nucleic acid sequence encoding a polypeptide of interest (such as a codon sequence).

In some examples, the generative model may be based on a model that can take as input a representation of a molecule, e.g., in space (such as 2D structure, 3D structure) and/or with respect to various properties and/or medias (such as audio, text, and/or visual media).

In some examples, the generative model may be based on a natural language processing model.

The generative model may be seen as a deep generative model. The generative model can be associated with a loss function to be optimized. The generative model can be used for unsupervised learning. In one or more example methods, the generative model comprises a generator and/or a discriminator.

In one or more example methods, the generative model is one or more of: a generative adversarial network model, a Wasserstein generative adversarial network model, a diffusion model, and a variational autoencoder. For example, the generative non-unidirectional model is one or more of: a generative adversarial network model, a Wasserstein generative adversarial network model, a diffusion model, and a variational autoencoder.

In some examples, the generative model is a generative adversarial network (GAN) model having at least one generator and at least one discriminator. For example, in the generative adversarial network model, during training, the generator is configured to generate training candidate biological sequences while the discriminator is configured to evaluate the training candidate biological sequences generated. For example, the generator maps from a latent space of input data to a data distribution of interest for biological sequences, while the discriminative network distinguishes training candidate biological sequences produced by the generator from a true distribution or real biological sequences. In other words, the generator may determine a joint probability of the training candidate biological sequence conditioned on the input biological sequence, while the discriminator may determine a conditional probability of the training candidate biological sequence being real or fake knowing the input biological sequence. For example, during training, the generator and discriminator can be optimized, e.g. simultaneously or alternatively in a loop manner. The generator is for example trained to produce data that is as realistic as possible, while the discriminator is trained to be as good as possible at distinguishing the generated candidate biological sequence(s) from the real biological sequence(s). This can be done using a loss function that measures the error of the discriminator. For example, during training, the generator improves in producing candidate biological sequences closer to the real ones, and the discriminator becomes better at distinguishing the generated data from the real data. This process can continue until the generator is able to produce data that is indistinguishable from the real data, at which point the GAN has reached an equilibrium.

In some examples, e.g., after training, the generator generates new data points, e.g., new candidate biological sequence(s). For example, the generator takes as input the input data (which can be a random noise signal and data indicative of an input biological sequence and/or a predetermined criterion) and tries to generate a candidate biological sequence that looks similar to those provided in the training data.

The Wasserstein generative adversarial network model (WGAN) can be seen as a variant of the GAN model. For example, the Wasserstein GAN allows the discriminator to output values that are not constrained to be between 0 and 1 and are therefore not to be understood as probabilities. For example, the discriminator is trained to maximize a difference in outputs on real biological sequences and generated candidate biological sequences, respectively. For example, the generator is trained to generate candidate biological sequences that maximizes the discriminator outputs. For example, WGAN uses a different loss function called the Wasserstein loss, which is based on the Wasserstein distance. For example, the Wasserstein loss function is derived to improve the stability of the training process. This can result in a faster and more robust optimization process.

In some examples, the generative model is based on a Wasserstein GAN with gradient penalty (WGAN-GP). For example, the Wasserstein loss to optimize may be expressed as e.g.,

$${min}_{G} {max}_{D ∈ 𝒟} {𝔼}_{{x}_{r} ∼ {p}_{r} ( {x}_{r} )} [ D ( {x}_{r} ) ] - {𝔼}_{z ∼ p ( z )} [ D ( G ( z ) ) ] \qquad ( 1 )$$

where the generator G simulates ‘fake’ examples from noisy inputs z (e.g., random variable), e.g.:

x_{f}=G(z) (2)

and where [symbol] denotes the set of 1-Lipschitz continuous functions and where D denotes the discriminator, xᵣ denotes the real biological sequences, pᵣ denotes a distribution of the real biological sequences (e.g., as referenced biological sequences for example in a database) and x_{f} denotes the candidate biological sequences generated by the generator G.

In some examples, the discriminator is trained and the generator is trained, simultaneously or alternatively. Training the discriminator may be called the ‘Critic’ for WGANs. For example, for training the discriminator, a gradient descent is performed on the following critic loss function, L, to update parameters φ of the discriminator network with the gradient ∇_{φ}L, e.g.,

$$L = \frac{1}{m} {\sum }_{i = 1}^{m} {D}_{φ} ( {x}_{{f}_{i}} ) - {D}_{φ} ( {x}_{{r}_{i}} ) + λ \cdot {( {‖ {∇}_{{\hat{x}}_{i}} {D}_{φ} ( {\hat{x}}_{i} ) ‖}_{2} - 1 )}^{2} \qquad ( 3 )$$

where the last term of the loss function is a regularization term, a gradient penalty that encourages 1-Lipschitz continuity of the critic, with respect to the inputs (e.g. input biological sequences); where, for samples i=1, . . . , m:

zᵢ~p(z)

xᵣ_{ᵢ}~pᵣ(xᵣ)

x_{f}_{ᵢ}~G_{θ}(zᵢ)

\hat{x}ᵢ=εᵢ·xᵣ_{ᵢ}+(1−εᵢ)·x_{f}_{ᵢ}

εᵢ~U[0,1], (Uniform distribution between 0 and 1)

where xᵣ_{ᵢ} and x_{f}_{ᵢ} are numerical representations of real and generated candidate biological sequences, respectively; where λ denotes a hyperparameter (e.g., an arbitrary value set for the domain of application),
where p(z) is a multivariate normal distribution with a diagonal covariance matrix and pᵣ(xᵣ) is the distribution of the real examples of input biological sequences, implicitly represented by the database.

In one or more examples, gradient can be updated to minimize the following critic loss function, e.g., used in training the Generator G with parameter θ. For example, during training of the generator, the gradient descent is performed using the gradient, e.g.:

$${∇}_{θ} \frac{1}{m} {\sum }_{i = 1}^{m} - {D}_{φ} ( {G}_{θ} ( {z}_{i} ) ) \qquad ( 4 )$$

where, for samples i=1, . . . , m:

zᵢ~p(z) (5)

For example, for the WGAN, the discriminator network (e.g., ‘Critic’) is not constrained to output values between 0 and 1 as in conventional GANs. A conditional WGAN-GP (CWGAN-GP) can be achieved using the disclosed functions by appending the conditional information to the input. In some examples, the generative model applies is the CWGAN-GP.

The diffusion model may aim at learning an underlying structure of the data set of input biological sequence (such as compatibility rules) by modelling how data points diffuse in a latent space associated with input biological sequences. For example, the diffusion model may be based on a Markov chain that performs a diffusion process by gradually adding noise to the training candidate biological sequence(s) and/or to the training output biological sequence(s). For example, another Markov chain is then trained to reverse this diffusion process, thus learning to generate candidate biological sequences from noise. The training may use variational inference (such as Bayesian inference). For example, the variational inference is used to derive a variational bound on the negative log likelihood over the training candidate biological sequences, which is a function of the forward and reverse processes.

The variational autoencoder (VAE) may be seen as a generative model using a prior distribution and a noise distribution for the input data associated with the input biological sequences. For example, the variational autoencoder is based on neural networks, such as an encoder neural network and a decoder neural network. For example, the encoder maps the input data to a latent space that corresponds to the variational distribution of the input data. In other words, for example, an encoder neural network maps the input data point to a latent representation, and a decoder neural network that maps the latent representation back to the original input data. For example, the encoder neural network takes the input data and maps it to a lower-dimensional latent representation. For example, the decoder neural network takes the latent representation and tries to reconstruct the original input data from the latent representation, for providing the candidate biological sequence. For example, the decoder neural network provides an opposite function to the encoder neural network, e.g. mapping from the latent space to a space of biological sequences, in order to generate a candidate biological sequence. In some examples, during training, the VAE is optimized to minimize the difference between the original input data and the reconstructed output (which is the candidate biological sequence). For example, this can be done using a loss function that measures the reconstruction error, which can be expressed as e.g.:

$$log {P}_{θ} ( x ) = {𝔼}_{z ∼ {q}_{φ} ( z \vert x )} [ log \frac{{P}_{θ} ( x \vert z ) \cdot {P}_{θ} ( z )}{{q}_{φ} ( z \vert x )} ] + K L [ {q}_{φ} ( z \vert x ) ‖ {P}_{θ} ( z \vert x ) ] \qquad ( 6 )$$

where x denotes a numerical representation of real biological sequences, distribution q_{φ}(z|x) denotes the encoder neural network (which may be seen as an approximation fo the true posterior for the encoder), KL denotes Kullback-Leibler divergence term, P_{θ}(x|z) denotes the decoder neural network, and P_{θ}(z|x) denotes an exact true posterior for the encoder. For example, after training, the decoder neural network can be used to generate candidate biological sequences. A conditional VAE can be achieved from the disclosed functions by appending the conditional information to the input. In some examples, the conditional VAE is applied to generate candidate biological sequences.

It may be appreciated that VAEs can learn to represent data in a continuous latent space, which allows them to generate new data points for candidate biological sequences that are similar to the training data. For example, this can be done by sampling from the latent space and passing the samples through the decoder network to generate the candidate biological sequence and related data as output data.

In some examples, the training data may include training data from a public database such as UniProt, and/or SwissProt and/or National Center for Biotechnology Information database and/or a Nucleotide Archive. In some examples, the training data may include training data from a private database.

In this disclosure, a “real” biological sequence may be seen as a referenced biological sequence, e.g. a biological sequence referenced in a database.

In one or more example methods, applying the generative model to the input data comprises partitioning the generative model into a plurality of generators. In one or more example methods, each generator of the plurality of generators is configured to determine, based on the input data, one or more candidate biological sequences for a subset of nucleotides and/or a subset of amino acids and a predetermined criterion.

In one embodiment, each generator among the plurality of generators is capable of generating candidate biological sequences from a certain subset of the entire sequence space. For example, each generator has a particular ‘focus’ on a subset of the sequence space. Preferably, this ‘focus’ is dictated by the value of the ‘predetermined criterion’. The example shown herein (i.e., regarding GC content) shows exactly this: each generator is defined by the predetermined criterion (here ‘threshold’ and aspect type, ‘GC/AT’).

In one embodiment, each generator, preferably depending on the predetermined criterion, then generates candidate biological sequences from a certain subset of the entire sequence space, e.g., with certain GC-content levels.

In some examples, the generative model can be partitioned into a plurality of generators, where each generator is associated with a subset of nucleotide and/or a subset of amino acids. For example, the generators can include generators for any combination of nucleotides or amino acids. For example, the generators can include a generator for aspects related to “GC” (with G being Guanine, and C being Cytosine), and/or a generator for aspects related to “AT” (with A being Adenosine, and T being Thymine). In some examples, each generator is associated with a subset of nucleotide and/or a subset of amino acid sequences and associated with a predetermined criterion. For example, the generators can include a generator for a content of subset of nucleotides or amino acids being higher than a threshold in generating the candidate biological sequence. For example, the generators can include a generator for a “GC” content higher than a threshold in generating the candidate biological sequence. For example, the generators can include a generator for a “GC” content lower than a threshold in generating the candidate biological sequence.

In one or more example methods, determining the candidate biological sequence comprises predicting (e.g., directly predicting, or indirectly predicting), using the generator, a compatibility of the candidate biological sequence with the host cell. In one or more example methods, determining the candidate biological sequence comprises determining the candidate biological sequence having a predicted compatibility meeting the predetermined criterion. In some examples, the method comprises determining for each candidate biological sequence whether the predicted compatibility meets the predetermined criterion. In some examples, in response to the predicted compatibility meeting the predetermined criterion, the candidate biological sequence is selected to be part of the biological sequence data provided as output. For example, when the compatibility is related to the GC content of the candidate biological sequence, the predetermined criterion is related to the GC content of the candidate biological sequence being below or above a threshold, and the disclosed technique determines and provides the candidate biological sequence which has the GC content of the candidate biological sequence being below or above a threshold.

In some examples, a data analysis method is applied to experimental validation data of the candidate biological sequences. As a result, properties of the candidate biological sequences associated with yield can be identified. In other words, the application of such a data analysis method leads to extracting learnings from experimental validation data. In some examples, such learnings are used as feedback to update the generative model by specifying a certain subset of generative models among a plurality of generative models. In some examples, specifying such a subset of generative models corresponds to specifying one or more values of a predetermined criterion of the generative model. For example, applying a data analysis method to experimental validation data results in learnings in the form of a specification of a predetermined criterion. Such a specification of a predetermined criterion may then be used as feedback to update the generative model, e.g., by specifying a subset of generative models among a plurality of generative models.

In some examples, the predetermined criterion is indicative of properties of a candidate biological sequence. For example, the property of a candidate biological is one or more of data indicative of observable descriptive properties, data indicative of machine derived predicted properties, and data indicative of experimentally measured properties. In one or more example methods, the predetermined criterion is based on one or more of: an embedding, a proportion of the set of nucleotides in the candidate biological sequence, a proportion of amino acids in the candidate biological sequence, a proportion of amino acids and/or nucleotides in certain subsets of the candidate biological sequence, a class of host cell, a host cell genus or species, a GC content of a host cell genome, a GC content of the candidate biological sequence, and a parameter associated with a property of the candidate biological sequence. For example, the class of the host cell can include a mammalian class for mammalian host cells, a bacterial class for bacterial host cells, a fungal class for fungal host cells, and/or a yeast class for a yeast host cell. For example, the parameter associated with a property of the candidate biological sequence includes temperature, and/or thermostability of the candidate biological sequence. For example, the predetermined criterion may be associated with a property of the candidate biological sequence that may depend on some context, such as the host and/or experimental conditions. In some examples, this property cannot be evaluated in isolation and this property is not an intrinsic property of the candidate biological sequence. An example of this could be ‘strength’ of a promoter sequence (e.g., low, medium, high strength) which depends on the actual host cell and/or cultivation conditions.

For example, the predetermined criterion may be an embedding from a deep learning model, e.g., a language model. Additionally or alternatively, the predetermined criterion is based on a subset of an embedding, or derived from an embedding.

For example, the parameter associated with a property of the candidate biological sequence includes binding properties, and/or enzyme activity.

For example, the parameter associated with a property of the candidate biological sequence includes physico-chemical properties of the candidate biological sequence, such as hydrophobicity, hydrophilicity, and charges. Such properties may be predicted and/or experimentally derived. For example, the parameter associated with a property of the candidate biological sequence includes physico-chemical properties, as described above, of certain subsets of the candidate biological sequence.

For example, an embedding could come from a large language model (LLM).

In one or more examples, such a parameter of the candidate biological sequence could be derived from an embedding. It may be noted that such an embedding could be any numerical representation, possibly random, that only makes sense when applying another learning task to it.

In one or more example methods, the method comprises training the generative model based on a training set of biological sequences. In one or more example methods, the training set of biological sequences includes training data indicative of one or more biological sequences related to the host cell, such as biological sequences that are endogenous and/or experimentally referenced. In other words, the training data is representative of training set of biological sequences. For example, the training set can be seen as a set of training biological sequences.

In one or more example methods, the training set of biological sequences is homologous to the genus of the host cell, preferably homologous to the species of the host cell. In one or more example methods, the training set of biological sequences is heterologous to the genus of the host cell, preferably heterologous to the species of the host cell.

The training set and/or training data can be obtained from a database, such as a database referencing the tree of life, such as a database referencing a genus of the host cell, and/or a species of a genus of the host cell.

For example, during training of the generator, the generator aims at deceiving the discriminator. The training may be based on providing training data to the generative model until acceptable accuracy is reached. The training set may be seen as providing a set of the conditional variables of the generative model. In one or more example methods, the training data comprises training input data indicative of one or more of: an amino acid sequence of a polypeptide of interest, a nucleic acid sequence encoding a polypeptide of interest, a control sequence, a nucleic acid sequence encoding a control sequence, properties of the candidate biological sequence. In other words, the training input data can be seen as input data used for training. In one or more example methods, the training data comprises training output data indicative of one or more of: a control sequence, a nucleic acid sequence encoding a control sequence, an amino acid sequence of a polypeptide of interest, and a nucleic acid sequence encoding a polypeptide of interest. In other words, the training input data can be seen as output data (e.g. candidate biological sequences) used for training. Alternatively, the training output data can be seen as output data (e.g. candidate biological sequences). In one or more example methods, the training input data is paired with the training output data. In some examples, the training input data is paired with the training output data in such a way that these together correspond to a pair referenced in a database. For example, training input data and training output data can be referenced as a pair if these are seen together in nature. For example, when the training input data contains an amino acid sequence of a polypeptide of interest and training output data contains a control sequence it is understood that the control sequence is an observed ‘partner’ sequence to the amino acid sequence. For example, when the training input data contains data indicative of properties of the candidate biological sequence (e.g., one or more predetermined criterion) it is understood that these properties are associated with the corresponding training output data. For example, a property of training output data could be its observed ‘GC’ content, e.g., where the training output data contains a nucleic acid sequence encoding a control sequence. In some examples, the training set can be augmented based on experimental data and candidate biological sequences that have been validated by experimental data. The candidate biological sequences may not be native biological sequences. In some examples, the candidate biological sequence may be a native biological sequence.

In one embodiment “training input data” means input data for modelling, e.g., during training of the model.

In one embodiment “training output data” means output data for modelling, e.g., during training of the model.

In one embodiment input data used for training comprises or consists of training input data and training output data.

In one or more example methods, training the generative model comprises predicting, using a discriminator taking as input the training set of biological sequences, and a training candidate biological sequence, a score indicative of the training candidate biological sequence being a referenced biological sequence. For example, when the generative model (such as a generative non-unidirectional model, such as a GAN) comprises a discriminator and a generator, the discriminator is used for training the generator and takes as input the training set of biological sequences, and a training candidate biological sequence, to predict a score indicative of the training candidate biological sequence being a referenced biological sequence. In some examples, the score indicative of the training candidate biological sequence being a referenced biological sequence may be seen as a likelihood that the training candidate biological sequence is referenced e.g., in a database referencing native biological sequences (e.g., referencing to sequences existing in nature, such as sequences from wildtype cells). In some examples, the score can estimate a likelihood that the training candidate biological sequence is compatible with the host cell. During training, the discriminator provides a conditional probability indicating how “real” the candidate biological sequence is as feedback to the generator.

In one or more example methods, the method comprises obtaining, from a test environment data repository, experimental data associated with the candidate biological sequence and the host cell.

In one or more example methods, the experimental data indicates a compatibility, e.g., a yield performance, of the candidate biological sequence associated with the host cell. In some examples, the yield performance can indicate an amount of product of interest secreted per volume unit of host cell broth or cultivation supernatant.

In one or more example methods, the method comprises validating the candidate biological sequence based on the experimental data. For example, the candidate biological sequence provided by the generative model is validated using the experimental data resulting from experiments of the candidate biological sequence with the host cell.

In one or more example methods, the method comprises selecting one or more generators based on the experimental data. It may be envisaged that the experimental data is used to select the generator(s) that are providing satisfactory experimental results in terms of yield etc.

In one or more example methods, the method comprises adapting the generative model based on the experimental data. For example, the experimental data and the resulting satisfactory candidate biological sequence(s) can be used to re-train the generative model (such as the GAN, and/or the one or more generators and/or one or more discriminators). For example, this allows GAN to be based on a quality of the nucleic acid sequence (e.g., secretion and/or translation from the host cell) in experimental settings.

The present disclosure provides an electronic device. The electronic device comprises a memory circuitry, a processor circuitry, and an interface. The electronic device is configured to perform any of the methods according to any of the disclosed methods.

The present disclosure provides a recombinant host cell comprising in its genome a first polynucleotide encoding a control sequence, and a second polynucleotide operably linked to the first polynucleotide encoding a polypeptide of interest, wherein the first or second polynucleotide is the candidate biological sequence obtained by the method disclosed herein.

The present disclosure provides a method of producing a polypeptide of interest, comprising cultivating the recombinant host cell disclosed herein under conditions conducive for production of the polypeptide of interest, and optionally recovering the polypeptide of interest.

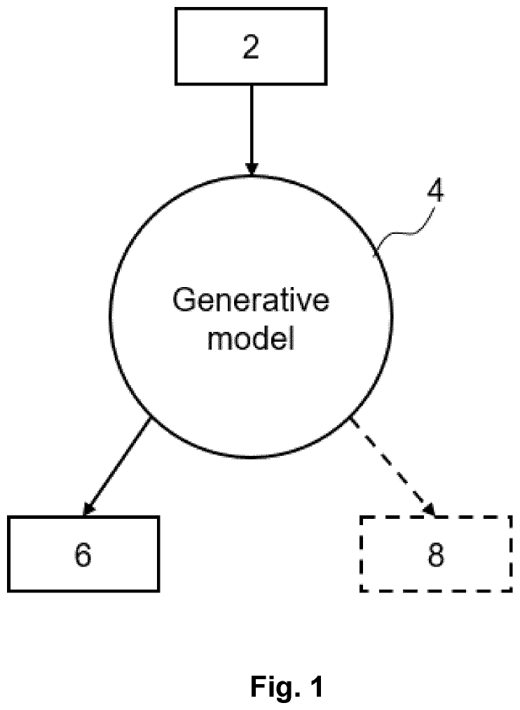

FIG. 1 is a diagram illustrating schematically an example implementation according to this disclosure. FIG. 1 shows example input data 2 indicative of an input biological sequence, a generative model 4, and biological sequence data 6 indicative of an example candidate biological sequence. The generative model 4 disclosed herein takes as input the input data 2 representative of the input biological sequence. For example, the input data 2 can be representative of one or more of: an amino acid sequence of a polypeptide of interest, a nucleic acid sequence encoding a polypeptide of interest, a control sequence, and a nucleic acid sequence encoding a control sequence.

A candidate biological sequence is determined based on the input data, e.g., by applying the generative model 4 to the input data 2. The generative model 4 provides biological sequence data 6 indicative of the candidate biological sequence and optionally biological sequence data 8 indicative of an additional candidate biological sequence. For example, biological sequence data can be representative of one or more of: a control sequence, a nucleic acid sequence encoding a control sequence, an amino acid sequence of a polypeptide of interest, and a nucleic acid sequence encoding a polypeptide of interest.

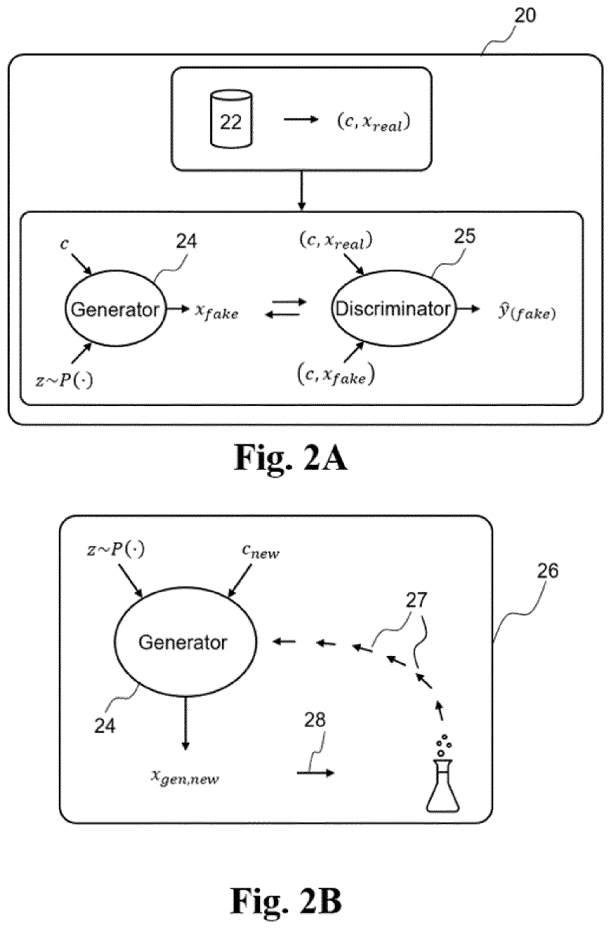

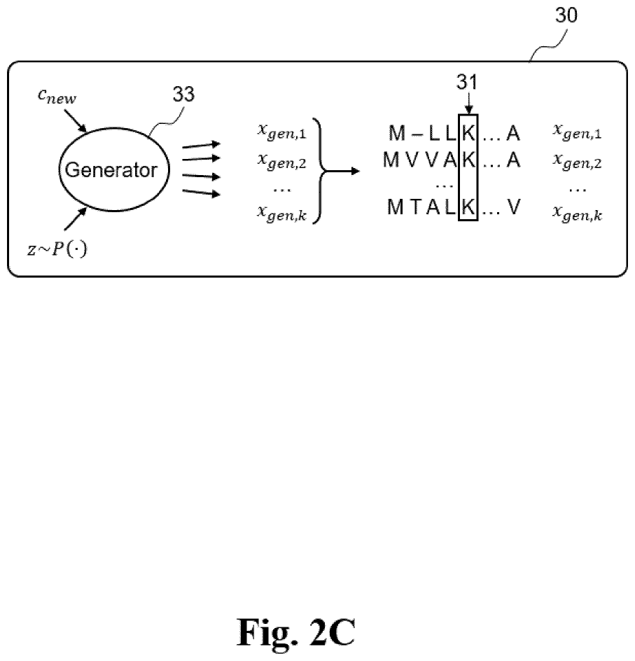

FIGS. 2A-C are diagrams illustrating schematically example implementations according to this disclosure. FIG. 2A shows an example implementation 20 for training and/or developing the generative model disclosed herein. FIG. 2A shows a database or data repository 22 for providing a training set of biological sequences (c, xᵣₑₐₗ) where xᵣₑₐₗ denotes the native partner biological sequence of c and c denotes an input biological sequence or a conditional variable (used for the training). For example, xᵣₑₐₗ denotes an actual signal peptide belonging to a protein c. Additionally, or alternatively, c comprises one or more predetermined criteria.

The training set is used for training the generative model. For example, the generative model comprises a generator 24 and a discriminator 25.

The generator 24 takes as input: (i) training data including data indicative of a biological sequence c (such as one or more of: an amino acid sequence of a polypeptide of interest, a nucleic acid sequence encoding a polypeptide of interest, a control sequence, and a nucleic acid sequence encoding a control sequence), optionally c further comprises one or more predetermined criterion, (ii) a random variable z in the latent space of biological sequences from 22 (e.g., correlating an input position of the input biological sequence with an output position of the training biological sequence), and (iii) a probability distribution P of the random variable z.

During the training phase, the generator 24 provides a training candidate biological sequence x_{fake} to the discriminator 25. x_{fake} denotes a training candidate biological sequence generated which does not necessarily exist in nature and is predicted to be compatible with c, e.g., by learning from xᵣₑₐₗ.

The discriminator 25 takes as input: (c, xᵣₑₐₗ), (c, x_{fake}) where (c, x_{fake}) denotes a fake pair in the sense that x_{fake} is a simulated partner of the conditional variable, i.e. the input biological sequence.

In a preferred embodiment, the discriminator 25 further takes as input one or more predetermined criterion.

The discriminator 25 predicts, based on the input, a score ŷ_{(fake)} indicative of the training candidate biological sequence being a referenced biological sequence, e.g., a likelihood that the training candidate biological sequence is a referenced biological sequence (e.g., in a database) and/or exists in nature.

The discriminator 25 may feedback ŷ_{fake} to the generator 24.

FIG. 2B shows an example implementation 26 illustrating the generator 24 during execution, e.g., after training. The generator 24 takes as input: (i) input data indicative of an input biological sequence c_{new} (such as a new input biological sequence to be studied, such as one or more of: an amino acid sequence of a polypeptide of interest, a nucleic acid sequence encoding a polypeptide of interest, a control sequence, and a nucleic acid sequence encoding a control sequence), and optionally c_{new} also comprises one or more predetermined criterion, (ii) a random variable z in the latent space of biological sequences (e.g., correlating an input position of the input biological sequence with an output position of the candidate biological sequence) and (iii) the probability distribution P of the random variable.

The generator 24 determines a candidate biological sequence x_{gen, new} which is predicted to be compatible with c_{new} and provides biological sequence data indicative of the candidate biological sequence.

The biological sequence data is provided in 28 for experiments to test the compatibility and/or performance of the candidate biological sequence.

The experimental data can be used to validate the candidate biological sequence.

The experimental data can be used to select a generator amongst a plurality of generators.

The experiments result in experimental data that can be used in 27 to update, adapt and/or retrain the generator.

FIG. 2C shows an example implementation 30 illustrating a mode of action using the generator 33.

The generator 33 takes as input: (i) input data indicative of an input biological sequence in c_{new} (such as a new input biological sequence to be studied, such as one or more of: an amino acid sequence of a polypeptide of interest, a nucleic acid sequence encoding a polypeptide of interest, a control sequence, and a nucleic acid sequence encoding a control sequence), optionally c_{new} also comprises one or more predetermined criterion, (ii) a random variable z in the latent space of biological sequences (e.g., correlating an input position of the input biological sequence with an output position of the candidate biological sequence) and (iii) the probability distribution P of the random variable.

The generator 33 determines a plurality of candidate biological sequences X_{gen,1}, X_{gen,2}, . . . , X_{gen,k} (where k is a positive integer) which is predicted to be compatible with C_{new} and provides biological sequence data indicative of the candidate biological sequence.

The candidate biological sequences X_{gen,1}, X_{gen,2}, . . . , X_{gen,k} are illustrated in FIG. 2C showing an amino acid 31 present in each of the candidate biological sequences.

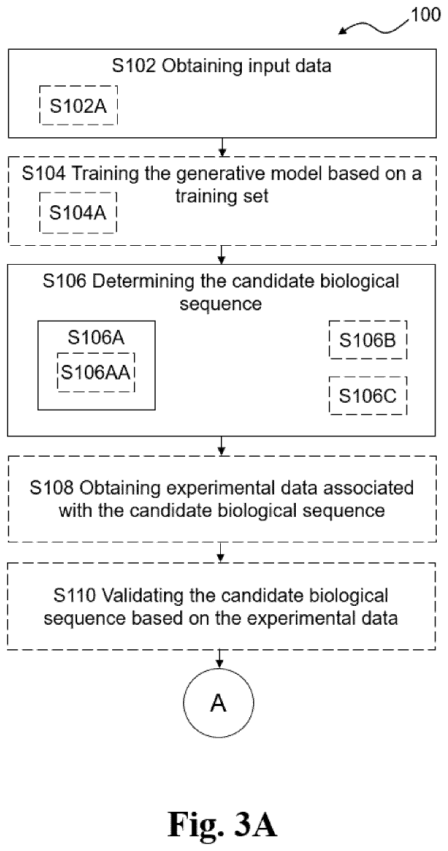

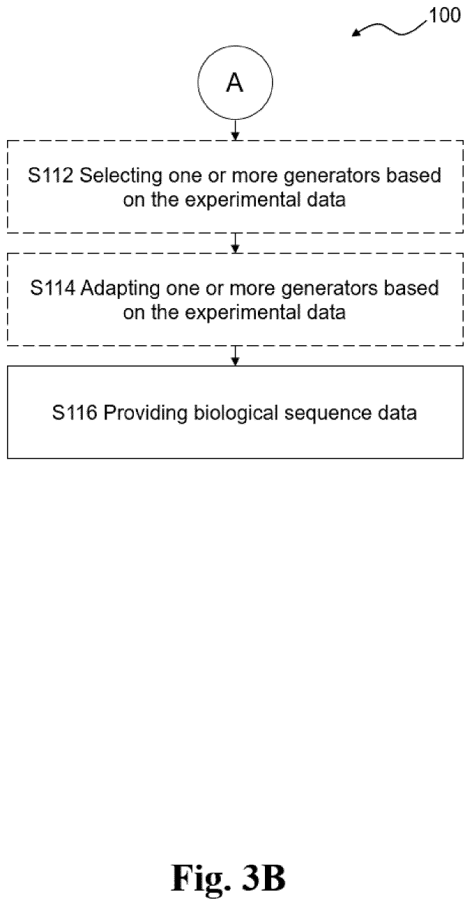

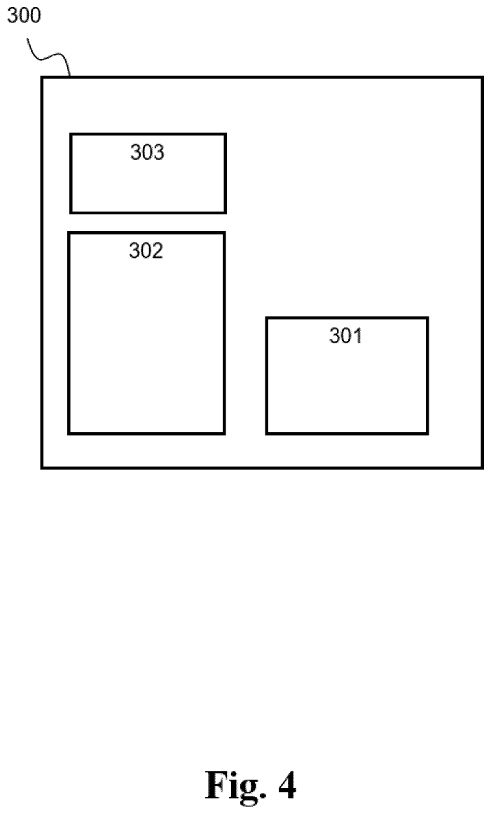

FIGS. 3A-B are a flow-chart illustrating an exemplary method 100, performed by an electronic device, for providing a candidate biological sequence according to this disclosure. The method 100 is performed by an electronic device, such as the electronic device disclosed herein, such as electronic device 300 of FIG. 4.

The method 100 comprises obtaining S102 input data indicative of an input biological sequence. In one or more example methods, obtaining input data indicative of an input biological sequence comprises obtaining (e.g., receiving and/or retrieving) the input data for the input biological sequence, optionally from a database and/or a memory of the electronic device.

The method 100 comprises determining S106 the candidate biological sequence by applying S106A a generative model to the input data. In some examples, the generative model is a generative non-unidirectional model. For example, the candidate biological sequence is determined based on the input data. For example, the candidate biological sequence is determined for compatibility with a host cell, e.g., targeting compatibility with a given host cell, and/or for increasing compatibility with the given host cell. Compatibility may be characterized by one or more compatibility rules which may be learned by the generative model disclosed herein.

In other words, the generative model is configured to characterize, and/or learn compatibility rules and/or compatibility patterns.

The method 100 comprises providing S116 biological sequence data indicative of the candidate biological sequence. For examples, the biological sequence data is transmitted to an external device, e.g., for experiments and/or for production. This is for example illustrated in FIG. 1 and FIGS. 2B-C.

In one or more example methods, the input biological sequence is one or more of: an amino acid sequence of a polypeptide of interest, a nucleic acid sequence encoding a polypeptide of interest, a control sequence, and a nucleic acid sequence encoding a control sequence.

In one or more example methods, the candidate biological sequence is one or more of: a control sequence, a nucleic acid sequence encoding a control sequence, an amino acid sequence of a polypeptide of interest, and a nucleic acid sequence encoding a polypeptide of interest.

In one or more example methods, the candidate biological sequence is extended by one or more nucleic acids or by one or more amino acids, thus providing an extended candidate biological sequence.

In one or more example methods, the candidate biological sequence is shortened by one or more nucleic acids or by one or more amino acids, thus providing a fragment candidate biological sequence.

In one or more example methods, the candidate biological sequence is fused to another biological sequence, thus providing a fusion polypeptide or a coding sequence encoding a fusion polypeptide.

In one or more example methods, the input biological sequence is an amino acid sequence of a polypeptide of interest and/or a nucleic acid sequence encoding a polypeptide of interest. In one or more example methods, the candidate biological sequence is a nucleic acid sequence increasing compatibility with a host cell.

In one or more example methods, the input biological sequence is an amino acid sequence of a polypeptide of interest and/or a nucleic acid sequence encoding a polypeptide of interest. In one or more example methods, the candidate biological sequence is a control sequence, e.g., an expression control sequence, and/or a nucleic acid sequence encoding a control sequence, e.g., an expression control sequence.

In one or more example methods, the input biological sequence is a control sequence, e.g., an expression control sequence, and/or a nucleic acid sequence encoding a control sequence, e.g., an expression control sequence. In one or more example methods, the candidate biological sequence is a nucleic acid sequence encoding a polypeptide of interest.

In one or more example methods, the generative model is one or more of: a generative adversarial network model, a Wasserstein generative adversarial network model, a diffusion model, and a variational autoencoder. For example, the generative non-unidirectional model is one or more of: a generative adversarial network model, a Wasserstein generative adversarial network model, a diffusion model, and a variational autoencoder.

In one or more example methods, the generative model comprises a generator and optionally a discriminator. In some examples, the generative model is a generative adversarial network (GAN) model having at least one generator and at least one discriminator. For example, in the generative adversarial network model, during training (e.g., illustrated in FIG. 2A), the generator is configured to generate training candidate biological sequences while the discriminator is configured to evaluate the training candidate biological sequences generated. For example, the generator maps from a latent space of input data to a data distribution of interest for biological sequences, while the discriminative network distinguishes training candidate biological sequences produced by the generator from the true data distribution. In other words, the generator may determine a joint probability of the training candidate biological sequence conditioned on the input biological sequence, while the discriminator may determine a conditional probability of the training candidate biological sequence being real or fake knowing the input biological sequence. In some examples, during training, the generator learns rules (e.g., compatibility rules) characterizing a relation between input biological sequence space and candidate biological sequence space. For example, during execution of the GAN (e.g., illustrated in FIG. 2B-C), the generator determines, based on the input data, one or more candidate biological sequences. For example, during execution of the GAN (e.g., illustrated in FIG. 2B-C), the generator applies the learned rules to the input data to generate the one or more candidate biological sequences.

In one or more example methods, applying S106A the generative model to the input data comprises partitioning S106AA the generative model into a plurality of generators. In one or more example methods, each generator of the plurality of generators is configured to determine, based on the input data, one or more candidate biological sequences for a subset of nucleotides and/or a subset of amino acids and a predetermined criterion. For example, the generators can include generators for any combination of nucleotides or amino acids. For example, the generators can include a generator for aspects related to “GC”. For example, the generator for “GC” determines candidate biological sequences that can include particular levels of GC. For example, the generators can include a generator for a content of “GC” being higher than a threshold, which determines candidate biological sequences that show a content of “GC” higher than the threshold. For example, the generators can be used when the input biological sequence is a control sequence (such as a signal peptide) and the candidate biological sequence is a nucleic acid sequence encoding a polypeptide of interest (such as a codon).

In one or more example methods, determining S106 the candidate biological sequence comprises predicting S106B, using the generator, a compatibility of the candidate biological sequence with the host cell. In one or more example methods, determining S106 the candidate biological sequence comprises determining S106C the candidate biological sequence having a predicted compatibility meeting the predetermined criterion. In some examples, the method comprises determining for each candidate biological sequence whether the predicted compatibility meets the predetermined criterion (such as showing a proportion of a set of nucleotides in the candidate biological sequence higher than a threshold). In some examples, in response to the predicted compatibility meeting the predetermined criterion, the candidate biological sequence is selected to be part of the biological sequence data provided as output.

In one or more example methods, the predetermined criterion is based on one or more of: a proportion of the set of nucleotides in the candidate biological sequence, a proportion of amino acids in the candidate biological sequence, a proportion of amino acids and/or nucleotides in certain subsets of the candidate biological sequence, a class of host cell, a host cell genus or species, a GC content of a host cell genome, a GC content of the candidate biological sequence, and a parameter associated with a property of the candidate biological sequence. In some examples, in response to a proportion of a set of nucleotides in the candidate biological sequence being higher than a threshold (thereby having predicted compatibility meeting the predetermined criterion), the candidate biological sequence is selected to be part of the biological sequence data provided as output. In some examples, in response to a proportion of a set of nucleotides in a certain subset of the candidate biological sequence being higher than a threshold (thereby having predicted compatibility meeting the predetermined criterion), the candidate biological sequence is selected to be part of the biological sequence data provided as output. In some examples, in response to the candidate biological sequence showing physico-chemical properties satisfying a condition (thereby having predicted compatibility meeting the predetermined criterion), the candidate biological sequence is selected to be part of the biological sequence data provided as output. In some examples, in response to the candidate biological sequence being in a predetermined class of host cell (thereby having predicted compatibility meeting the predetermined criterion), the candidate biological sequence is selected to be part of the biological sequence data provided as output. In some examples, in response to the candidate biological sequence being a host cell genus or species (thereby having predicted compatibility meeting the predetermined criterion), the candidate biological sequence is selected to be part of the biological sequence data provided as output. In some examples, in response to the candidate biological sequence showing a parameter associated with a property of the candidate biological sequence satisfying a condition (thereby having predicted compatibility meeting the predetermined criterion), the candidate biological sequence is selected to be part of the biological sequence data provided as output.

In one or more example methods, the method comprises training S104 the generative model based on a training set of biological sequences. In one or more example methods, the training set of biological sequences includes training data indicative of one or more biological sequences related to the host cell. An example training of the generative model is provided in FIG. 2A. In one or more example methods, the training set of biological sequences or parts thereof is heterologous to the genus of the host cell, preferably heterologous to one or more species of the host cell. In a preferred example method, a subset of the training set of biological sequences is heterologous to the genus of the host cell.

In one or more example methods, the training data comprises training input data indicative of one or more of: an amino acid sequence of a polypeptide of interest, a nucleic acid sequence encoding a polypeptide of interest, a control sequence, and a nucleic acid sequence encoding a control sequence. In one or more example methods, the training data comprises training output data indicative of one or more of: a control sequence, a nucleic acid sequence encoding a control sequence, an amino acid sequence of a polypeptide of interest, and a nucleic acid sequence encoding a polypeptide of interest.

In one or more example methods, training S104 the generative model comprises predicting S104A, using a discriminator taking as input the training set of biological sequences, and a training candidate biological sequence, a score indicative of the training candidate biological sequence being a referenced biological sequence. For example, during training, the score is predicted using a discriminator taking as input the training set of biological sequences, and a training candidate biological sequence. In some examples, the training candidate biological sequence can be a real biological sequence or a fake biological sequence. A real biological sequence is a sequence that exists in nature. A fake biological sequence does not necessarily exist in nature or is not referenced in a database of experimentally validated sequences. This is for example illustrated in FIG. 2A. In some examples, the discriminator evaluates the training candidate biological sequences generated by distinguishes the training candidate biological sequence produced by the generator from the true distribution.

In one or more example methods, the method comprises obtaining S108, from a test environment data repository, experimental data associated with the candidate biological sequence and the host cell. In one or more example methods, the experimental data indicates a yield performance of the candidate biological sequence associated with the host cell. The experimental data can be obtained from experimental setting as illustrated in FIG. 2B.

In one or more example methods, the method comprises validating S110 the candidate biological sequence based on the experimental data. For example, validation of the candidate biological sequence is performed using the experimental data resulting from experiments of the candidate biological sequence with the host cell. In one or more example methods, the method comprises selecting S112 one or more generators based on the experimental data. In one or more example methods, the method comprises adapting S114 the generative model (such as one or more generators) based on the experimental data. For example, adapting S114 comprises retraining the generative mode using results, and/or validation based on the experimental data.

FIG. 4 shows a block diagram of an exemplary electronic device 300 according to the disclosure. The electronic device 300 comprises a memory circuitry 301, a processor circuitry 302, and an interface 303. The electronic device 300 is configured to perform any of the methods disclosed in FIGS. 3A-B. In other words, the electronic device 300 is configured for providing a candidate biological sequence.

The electronic device 300 is configured to obtain (e.g., via processor circuitry 302 and/or interface 303) input data indicative of an input biological sequence.

The electronic device 300 is configured to determine (e.g., via processor circuitry 302) the candidate biological sequence by applying a generative non-unidirectional model to the input data.

The electronic device 300 is configured to provide (e.g., via processor circuitry 302 and/or interface 303) biological sequence data indicative of the candidate biological sequence.

In some examples, the electronic device 300 is a server device configured to communicate with a user device, via a wired and/or a wireless system. For example, the electronic device 300, as a server device, is configured to receive input data indicative of an input biological sequence from a user device, such as a client device. For example, the electronic device 300, as a server device, is configured to provide (e.g., transmit) biological sequence data indicative of the candidate biological sequence.

In some examples, the electronic device 300 is a user device configured to obtain input data indicative of an input biological sequence from user input. In some examples, the user device is a portable electronic device, such as a laptop.

In some examples, the processor circuitry 302 comprises one or more processors configured to perform any of the methods disclosed in FIGS. 3A-B.

The processor circuitry 302 is optionally configured to perform any of the operations disclosed in FIGS. 3A-B (such as any one or more of: S102, S102A, S104, S104A, S106, S106A, S106AA, S106B, S106C, S108, S110, S112, S114, S116). The operations of the electronic device 300 may be embodied in the form of executable logic routines (e.g., lines of code, software programs, etc.) that are stored on a non-transitory computer readable medium (e.g., the memory circuitry 301) and are executed by the processor circuitry 302).

Furthermore, the operations of the electronic device 300 may be considered a method that the electronic device 300 is configured to carry out. Also, while the described functions and operations may be implemented in software, such functionality may as well be carried out via dedicated hardware or firmware, or some combination of hardware, firmware and/or software.

The memory circuitry 301 may be one or more of a buffer, a flash memory, a hard drive, a removable media, a volatile memory, a non-volatile memory, a random-access memory (RAM), or other suitable device. In a typical arrangement, the memory circuitry 301 may include a non-volatile memory for long term data storage and a volatile memory that functions as system memory for the processor circuitry 302. The memory circuitry 301 may exchange data with the processor circuitry 302 over a data bus. Control lines and an address bus between the memory circuitry 301 and the processor circuitry 302 also may be present (not shown in FIG. 4). The memory circuitry 301 is considered a non-transitory computer readable medium.

The memory circuitry 301 may be configured to store input data, input biological sequence, candidate biological sequence, biological sequence data, generative model, a proportion of the set of nucleotides in the candidate biological sequence, a class of host cell, a host cell genus or species, a GC content of a host cell genome, a GC content of the candidate biological sequence, a parameter associated with a property of the candidate biological sequence, training set of biological sequences, a discriminator, experimental data, in a part of the memory.

The present disclosure provides a computer readable storage medium. The computer readable storage medium stores one or more programs, the one or more programs comprising instructions, which when executed by an electronic device cause the electronic device to perform any of the methods according to the disclosed methods.

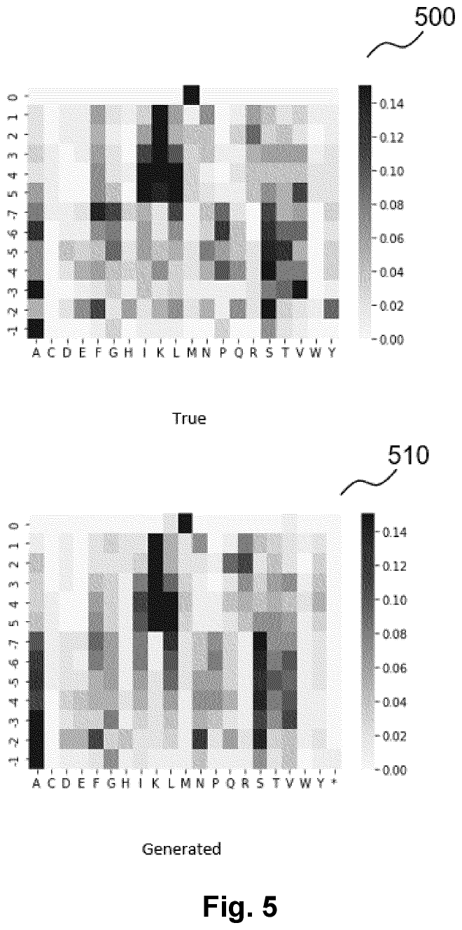

FIG. 5 shows a comparison between true position-wise amino acid frequencies 500 and position-wise amino acid frequencies generated 510 according to the disclosed method. It is to be noted that only parts of the amino acid sequence are shown. For example, only the 6 most N terminal residues and the 7 most C terminal residues are shown on FIG. 5. FIG. 5 shows similarities between true and generated position-wise amino acid frequencies.

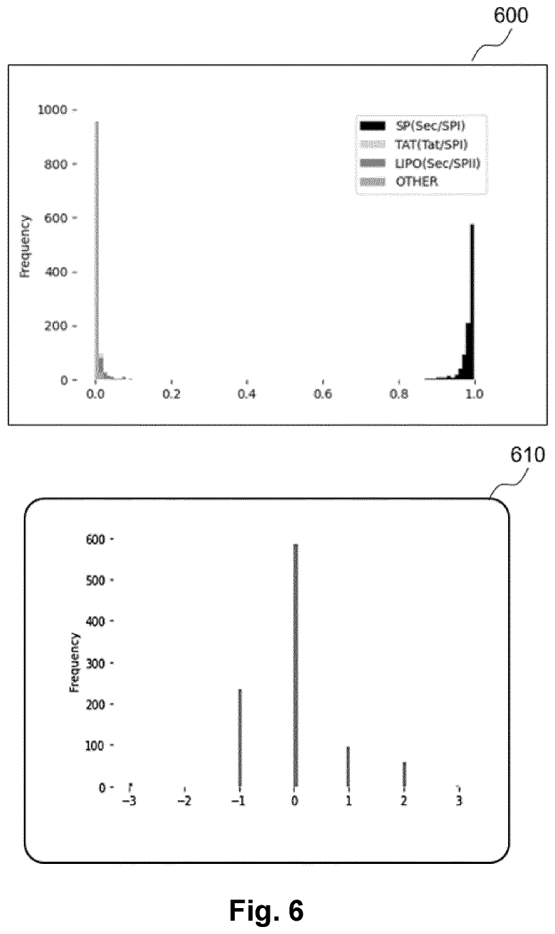

FIG. 6 is an illustration of example results 600, 610 according to this disclosure.

Results 600 illustrate confidence distributions per class of secretion pathways (and ‘other’) vs. frequencies, when testing the generated candidate biological sequences with SignalP 5.0. In other words, the classes illustrated can be of secretion pathways or other.

The results 600 show that the disclosed method predicts generated signal peptides (which are translated from DNA sequence for a Savinase protease) to be of intended class, i.e. secretion pathway, ‘SP(Sec/SPI)’, with high probability (e.g. above 0.8). The results 600 show that the generated signal peptides are determined with low probability (e.g. below 0.1) of being of the class “TAT(TAT/SPI)” or of class “LIPO(Sec/SPII)” or of other classes.

Results 610 illustrate a cleavage site mismatch distribution between where the candidate biological sequence was generated to be localized vs. where SignalP predicts the candidate biological sequence to be localized. Thus, this shows where SignalP detects the cleavage site relative to where it was generated to be for a candidate biological sequence generated by the disclosed technique. A value of 0 indicates no mismatch while values of e.g., +/−1 indicates a mismatch of 1 position either towards the N- or C-terminal side relative to the cleavage site.

The results 610 show that the majority of the generated signal peptides by the disclosed method are identified with the ‘correct’ cleavage site (shown as mismatch=0 in results 610).

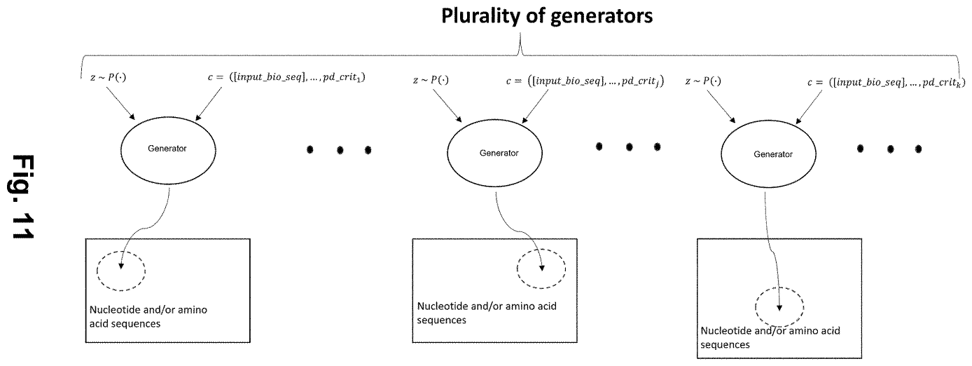

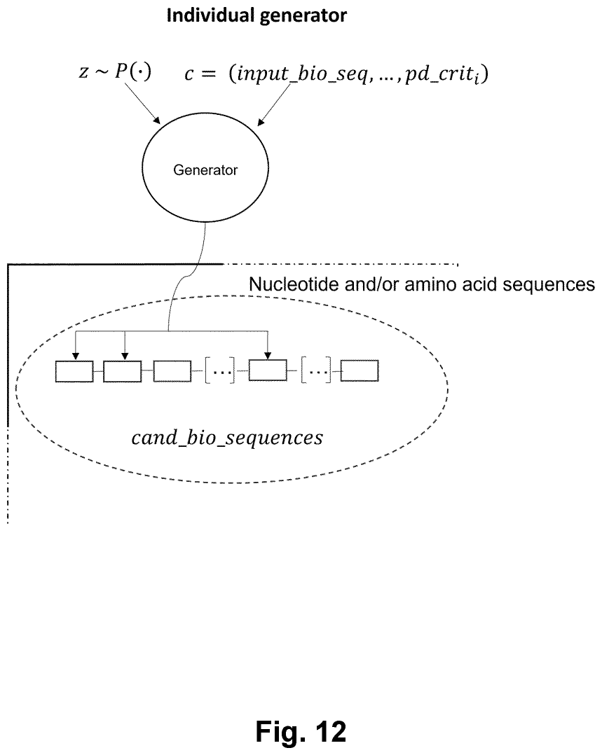

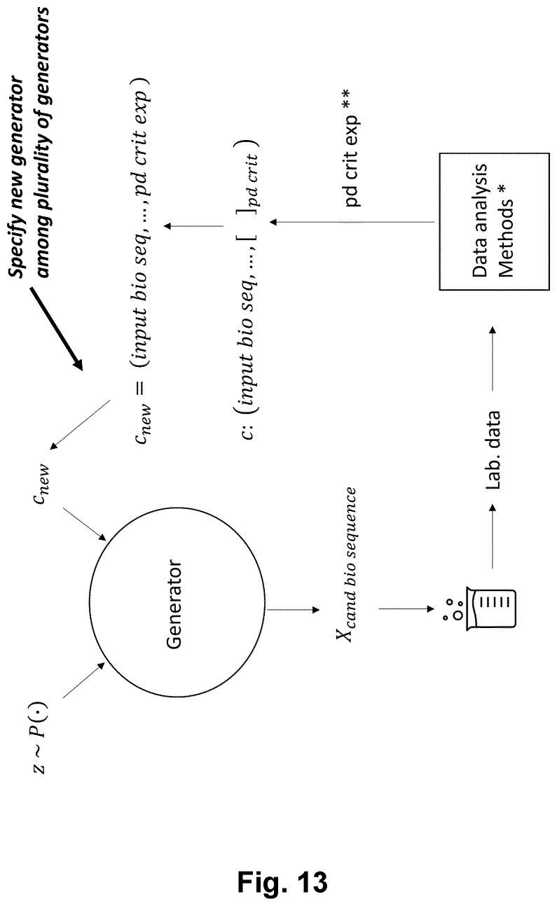

FIG. 11 shows one embodiment of the invention, comprising partitioning of a generative model into a plurality of generators. As shown in FIG. 11, each generator is configured to determine, based on the input data, one or more candidate biological sequences for a subset of nucleotides and/or a subset of amino acids and a predetermined criterion. In some examples, each such generator is configured to generate from a certain subspace of candidate biological sequences (thereby having predicted compatibility meeting the predetermined criterion). In some examples, for each such generator, a subset of nucleotides and/or a subset of amino acids will be under the control of the generator. In other words, and as shown in FIG. 12 representing one embodiment of the invention, for each such generator a subset of nucleotides and/or a subset of amino acids will depend on the input biological sequence and/or a predetermined criterion specifying the generator. In some examples, the partitioning of a generative model into a plurality of generators is defined prior to and/or as part of the training process. In some examples, each generator among a plurality of generators is specified by a predetermined criterion. In some examples, a result of the training process is a configuration of each generator among a plurality of generators to determine, based on the input data, one or more candidate biological sequences for a subset of nucleotides and/or a subset of amino acids and a predetermined criterion (FIG. 11). FIG. 13 shows one embodiment of the invention, i.e., navigating in a plurality of generators using experimentally guided iterations. In FIG. 13 the method is comprising a generator among a plurality of generators determining one or more candidate biological sequences (X_{cand bio sequence}) having a predicted compatibility meeting the predetermined criterion, for a given input biological sequence (input bio seq). ** in FIG. 13 indicates experimentally derived predetermined criterion, iteration k, which suggested predetermined criterion can be input to the generator for the next iteration. * in FIG. 13 indicates unspecified data analysis methods, but can for example be a downstream analysis method (e.g. Random Forrest, or Neural Network).

In one example, the data analysis method comprises an outlier detector and/or is derived from an outlier detector.

In one or more examples, the candidate biological sequences are validated based on experimental data in a host. In one or more examples, the experimental data indicates a compatibility, e.g., a yield performance. In some examples, a data analysis method can be applied to the experimental validation data of the candidate biological sequences. In some examples, this may lead to the identification of properties of the candidate biological sequences associated with yield. In some examples, such learnings can be used as feedback to update the generative model by specifying a certain subset of generative models among a plurality of generative models. In some examples, specifying such a subset of generative models corresponds to specifying a predetermined criterion of the generative model. In other words, the outcome of applying a data analysis method to experimental validation data could be learnings in the form of a specification of a predetermined criterion. Such a specification of a predetermined criterion can be used as feedback to update the generative model, by specifying a subset of generative models among a plurality of generative models. In some examples, and as shown in FIG. 14, this process unlocks the potential of screening by facilitating a data-driven prioritization of which experiments to conduct among the set of all experiments.

In an exemplary method, the generative model determines a candidate biological sequences (e.g. signal peptide amino acid sequences), from input biological sequences (e.g. mature polypeptide of interest) and a predetermined criterion (e.g. number of occurrences of amino acids in the following specified subsequences of the signal peptides: K/R in the 6 first amino acids, Y/W/F after the first 6 and before the 3 amino acids, total number of P. All positions are counted from the N-terminal end).

In an exemplary method, experimental validation is applied to candidate biological sequences determined from input biological sequences. In an exemplary method, a data analysis method is applied to experimental validation data to specify values of a predetermined criterion for a new round of generation.

In an exemplary method, the relevant training data is identified by specifying a certain subset of the tree of life, i.e., related to the host (e.g., from the same genus such as Bacillus).

In an exemplary method, selecting the relevant training data comprises selecting biological sequences, from this subset of the tree of life, meeting a specified category (e.g. amino acid sequences having a predicted signal peptide, according to SignalP).

In an exemplary method, the training input data comprises the set of mature polypeptides and values (e.g. a threshold) according to the predetermined criterion.

In an exemplary method, the training output data comprises the set of signal peptide amino acids sequences.

In an exemplary method, the training input data and training output data is paired. In other words, any signal peptide in the training output data is a partner sequence with the respective mature polypeptide of interest and the value of the predetermined criterion. In other words, the signal peptide amino acid sequence is paired with the mature polypeptide of interest in the sense that these are referenced as a pair in a database (e.g., a database containing wild type biological sequences). Also, the signal peptide amino acid sequence is paired with a value of the predetermined criterion in such a way that the value of the predetermined criterion is indicative of properties of the signal peptide amino acid sequence (e.g. number of occurrences of amino acids as specified above, in the signal peptide amino acid sequence). In other words, the signal peptide in the training output data is paired with a predetermined criterion that is indicative of observed and/or calculated and/or known properties of the signal peptide.

In one or more examples, the generative model is trained using such training data.

In some examples, training the generative model comprises selecting at random (with probability between 0 and 1, e.g. 0.5) whether or not to randomly change the value of the predetermined criterion, for any given training input/training output pair.

In an exemplary method, a plurality of generators is achieved from the set of all possible values for the predetermined criterion (e.g. as defined in the training input data).

In an exemplary method, biological sequences in the training data are numerically encoded in such a way that these are indicative of the biological sequences. In an exemplary method, a numerical encoding could be in the form of a one-hot encoding. In some examples, such a one-hot encoding could be achieved by representing each amino acid (in an amino acid sequence) by a numerical vector. In some examples, the size of such a numerical vector could be equal to the size of a prespecified dictionary, such as a dictionary mapping amino acids to 1-20 (in bijective fashion) as well as mapping a start and end token to 0 and 21, respectively. A one-hot encoding of any character (e.g. amino acid, start tokens, stop tokens) can then be achieved by assigning the value of zero in each position in the numerical vector, except for the position corresponding to the value of the character in question (an integer value, as defined by the dictionary above), at which the value of one is assigned. In some examples, the one-hot encoding of an entire amino acid sequence is achieved by concatenating all one-hot encodings for all positions in the sequence. The sequence of the concatenation is done to reflect the sequence of the characters of the biological sequence in question. In some examples, the biological sequences are encoded by instead representing each position by the corresponding value (an integer) of the above dictionary (e.g. any occurrence of ‘A’ is mapped to the value of the above dictionary corresponding to ‘A’ e.g. the integer 2). As before the sequence is encoded sequentially to reflect the biological sequence in question. In some examples, such an integer encoding could be combined with the presence of an embedding layer in neural network. It may be noted that, in some examples, biological sequences in training input data and training output data might be encoded by different strategies (e.g., biological sequences in the training input data might be encoded by the integer encoding strategy and the biological sequences in the training output data might be encoded by the one-hot encoding strategy).

In an exemplary method, a Wasserstein Generative Adversarial Network (WGAN) was trained to generate training output biological sequences from training input biological sequences and a predetermined criterion.

In an exemplary method, a training of a WGAN to generate training output biological sequences from input biological sequences can be seen as learning to generate sequences according to certain compatibility rules related to the host, since the training data comprises sequences related to the host.

In an exemplary method, after the end of training, the generative model is configured to determine candidate biological sequences from new input biological sequences and a predetermined criterion, in accordance with learned compatibility rules related to the host.

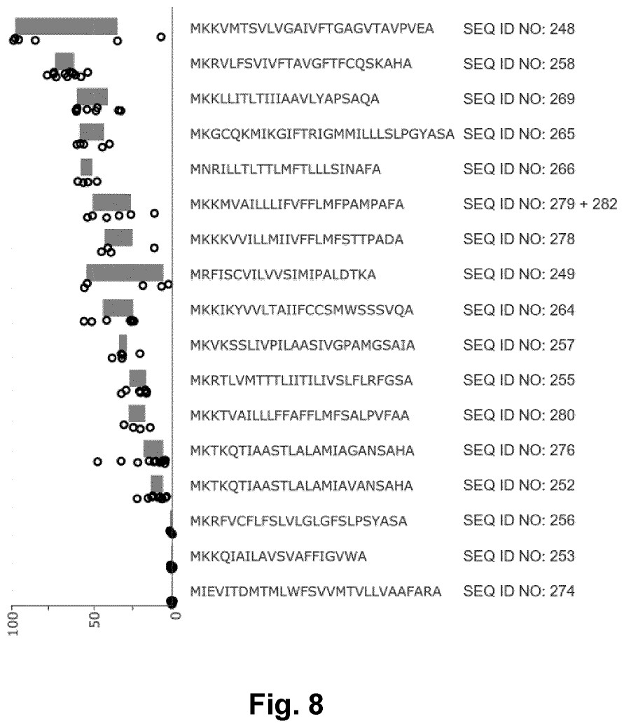

In an exemplary method, the new input biological sequence is a polypeptide of interest (e.g., the generated signal peptide sequences shown in FIG. 8).

In one or more examples, after end of training, specifying a value of a predetermined criterion is applied to control the process of determining candidate biological sequences from new input biological sequences.

In one or more examples, after end of training, the predetermined criterion is one or more of: a user defined value, an experimentally derived value, a value predicted from candidate biological sequences (from one or more previous rounds of generation) and its associated experimental validation data, a stochastically selected value.

In some examples, a first round of generation could be achieved by stochastically assigning the values of the predetermined criterion (e.g., by stochastically selecting number of occurrences of the amino acids as described above) to determine one or more candidate biological sequences, from an input biological sequence of interest (e.g. as shown in FIG. 8).

In some examples, the one or more candidate biological sequences can be experimentally validated in the context of e.g., polypeptide yield.

In an exemplary method, a data analysis method (e.g., a random forest classifier) is applied to extract learnings from experimental validation data. In an exemplary method, such learnings comprise a specification of a predetermined criterion associated with yield (e.g., number of Prolines, P, in the signal peptide amino acid sequences).

In an exemplary method, the specification of a predetermined criterion is used to adapt the generative model by specifying a subset of generators among a plurality of generators. In some examples, specifying a subset of generators means selecting a subset of generators. In some examples, specifying a subset of generators means prioritizing a subset of generators (e.g. a subset is used with a higher probability and/or weight, compared to the remaining set).

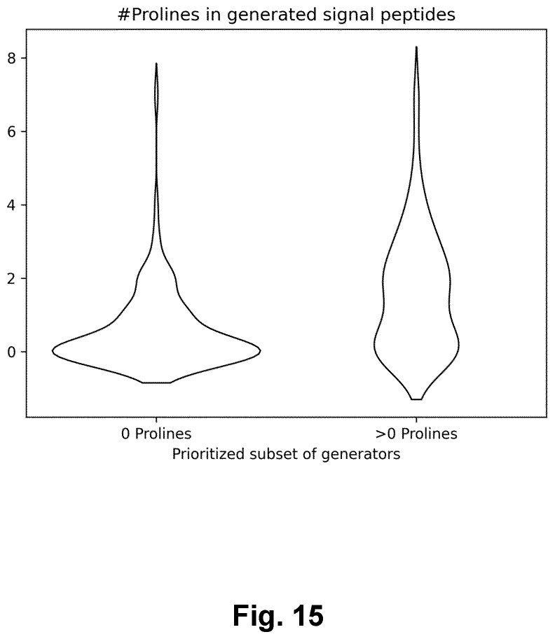

In an exemplary method, a new round of generation is achieved by prioritizing a subset of generators, namely those with high values of the predetermined criterion related to Proline content (e.g. higher than or equal to 1). FIG. 15 shows a violin plot illustrating how prioritizing different generators (i.e. those focusing on 0 Prolines vs more than 0 Prolines) leads to differences in the Proline content among generated signal peptides.

### Candidate Biological Sequence May be a Polypeptide

The present invention also relates to polypeptides, e.g. control sequences or polypeptide of interest, obtained using the method of the invention. In an aspect, the invention relates to polypeptides which are variants of a input biological sequence, either on nucleic acid sequence level or on amino acid sequence level, selected from the group consisting of:
(a) a polypeptide having at least 60% sequence identity to the amino acid sequence of the input biological sequence;
(b) a nucleic acid sequence encoding a polypeptide having at least 60% sequence identity to the nucleic acid sequence of the input biological sequence;
(c) a polypeptide having at least 60% sequence identity to the amino acid sequence of the input biological sequence of the mature polypeptide;
(d) a polypeptide encoded by a polynucleotide having at least 60% sequence identity to the mature polypeptide coding sequence of the input biological sequence;
(e) a polypeptide derived from the biological input sequence, a mature polypeptide of interest of the biological input sequence by substitution, deletion or addition of one or several amino acids;
(f) a polypeptide derived from the polypeptide of (a), (b), (c), (d) or (e) wherein the N- and/or C-terminal end has been extended by the addition of one or more amino acids; and
(g) a fragment of the polypeptide of (a), (b), (c), (d), or (e).

In one embodiment the polypeptide is an amylase.

In one embodiment the polypeptide is a chaperone.

In one embodiment the polypeptide is a protease.

In another embodiment the polypeptide is a cutinase.

In one embodiment the polypeptide is a signal peptide.

In an aspect, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the input biological sequence or a mature polypeptide thereof.

In another aspect, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the biological input sequence.

In another aspect, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 292.

In another aspect, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to any one of SEQ ID NO: 248 to 289.

The polypeptide may have an N-terminal and/or C-terminal extension of one or more amino acids, e.g., 1-5 amino acids.

In some embodiments, the polypeptide is encoded by a polynucleotide having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of the biological input sequence.

In some embodiments, the polypeptide is encoded by a polynucleotide having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 291.

In some embodiments, the polypeptide is encoded by a polynucleotide having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of any one of SEQ ID NO: 1 to 247.

In one embodiment, the candidate sequence comprises or encodes for an enzyme. The enzyme being selected from the list of a hydrolase, isomerase, ligase, lyase, oxidoreductase, or transferase, e.g., an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase, xylanase, or beta-xylosidase.

In another aspect, the polypeptide is derived from the input biological sequence by substitution, deletion or addition of one or several amino acids. In another aspect, the polypeptide is derived from a mature polypeptide of the input biological sequence by substitution, deletion or addition of one or several amino acids. In one aspect, the number of amino acid substitutions, deletions and/or insertions introduced into the polypeptide of the input biological sequence is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant molecules are tested for enzyme activity or binding activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labelling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide, and/or be inferred from sequence homology and conserved catalytic machinery with a related polypeptide or within a polypeptide or protein family with polypeptides/proteins descending from a common ancestor, typically having similar three-dimensional structures, functions, and significant sequence similarity. Additionally or alternatively, protein structure prediction tools can be used for protein structure modelling to identify essential amino acids and/or active sites of polypeptides. See, for example, Jumper et al., 2021, “Highly accurate protein structure prediction with AlphaFold”, Nature 596: 583-589.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

The polypeptide may be a fusion polypeptide.

In an aspect, the polypeptide is isolated.

In another aspect, the polypeptide is purified.

### Sources of Polypeptides to be Used as Biological Input Sequences

A biological input sequence of the present invention may be obtained from microorganisms of any genus. For purposes of the present invention, the term “obtained from” as used herein in connection with a given source shall mean that the polypeptide encoded by a polynucleotide is produced by the source or by a strain in which the polynucleotide of the invention has been inserted. In one aspect, the polypeptide obtained from a given source is secreted extracellularly.

In one aspect, the biological input sequence is obtained from a microbial cell, e.g., a prokaryotic cell or a fungal cell.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells. In an embodiment, the Bacillus cell is a Bacillus amyloliquefaciens, Bacillus licheniformis and Bacillus subtilis cell.

In one embodiment the biological input sequence is obtained from Alkalihalobacillus clausii.

For purposes of this invention, Bacillus classes/genera/species shall be defined as described in Patel and Gupta, 2020, Int. J. Syst. Evol. Microbiol. 70: 406-438.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

Methods for introducing DNA into prokaryotic host cells are well-known in the art, and any suitable method can be used including but not limited to protoplast transformation, competent cell transformation, electroporation, conjugation, transduction, with DNA introduced as linearized or as circular polynucleotide. Persons skilled in the art will be readily able to identify a suitable method for introducing DNA into a given prokaryotic cell depending, e.g., on the genus. Methods for introducing DNA into prokaryotic host cells are for example described in Heinze et al., 2018, BMC Microbiology 18:56, Burke et al., 2001, Proc. Natl. Acad. Sci. USA 98: 6289-6294, Choi et al., 2006, J. Microbiol. Methods 64: 391-397, and Donald et al., 2013, J. Bacteriol. 195(11): 2612-2620.

The host cell from which the biological input sequence is obtained may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). For purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell. In a preferred embodiment, the yeast host cell is a Pichia or Komagataella cell, e.g., a Pichia pastoris cell (Komagataella phaffii).

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell. In a preferred embodiment, the filamentous fungal host cell is an Aspergillus, Trichoderma or Fusarium cell. In a further preferred embodiment, the filamentous fungal host cell is an Aspergillus niger, Aspergillus oryzae, Trichoderma reesei, or Fusarium venenatum cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

The biological input sequence may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Davis et al., 2012, Basic Methods in Molecular Biology, Elsevier).

### Candidate Biological Sequence May be a Polynucleotide

The present invention also relates to polynucleotides obtained by the method of the invention.

The polynucleotide may be a genomic DNA, a cDNA, a synthetic DNA, a synthetic RNA, a mRNA, or a combination thereof. The polynucleotide may be cloned from a strain of any eukaryotic or procaryotic origin, or a related organism and thus, for example, may be a polynucleotide sequence encoding a variant of a polypeptide.

In an embodiment, the polynucleotide is a subsequence encoding a fragment of the biological input sequence.

In some embodiments, the candidate biological sequence is a DNA sequence having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to any one of SEQ ID NOs: 1 to 247.

In some embodiments, the candidate biological sequence is polynucleotide encoding a polypeptide having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to any one of SEQ ID NOs: 248 to 289.

The polynucleotide may also be mutated by introduction of nucleotide substitutions that do not result in a change in the amino acid sequence of the polypeptide, but which correspond to the codon usage of the host organism intended for production of the enzyme, or by introduction of nucleotide substitutions that may give rise to a different amino acid sequence. For a general description of nucleotide substitution, see, e.g., Ford et al., 1991, Protein Expression and Purification 2: 95-107.

In an aspect, the polynucleotide is isolated.

In another aspect, the polynucleotide is purified.

### Nucleic Acid Constructs

The present invention also relates to nucleic acid constructs comprising a polynucleotide (candidate biological sequence) generated with the method of the present invention. The polynucleotide may be operably linked to one or more control sequences that direct the expression of the coding sequence in a suitable host cell under conditions compatible with the control sequences. Alternatively, the polynucleotide may comprise or consist of a control sequence, e.g., an expression control sequence, or encode a control sequence, which is operably linked to one or more polypeptides of interest.

In one embodiment the polynucleotide comprises or consists of a signal peptide coding sequence.

In some embodiments, the polynucleotide comprises a polynucleotide sequence having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to any one of SEQ ID NOs: 1 to 247.

In some embodiments, the polynucleotide comprises a polynucleotide sequence encoding an polypeptide having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to any one of SEQ ID NOs: 248 to 289.

The polynucleotide may be manipulated in a variety of ways to provide for expression of the polypeptide. Manipulation of the polynucleotide prior to its insertion into a vector may be desirable or necessary depending on the expression vector. Techniques for modifying polynucleotides utilizing recombinant DNA methods are well known in the art.

### Promoters

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the polynucleotide of the present invention in a bacterial host cell are described in Sambrook et al., 1989, Molecular Cloning: A Laboratory Manual, Cold Spring Harbor Lab., NY, Davis et al., 2012, supra, and Song et al., 2016, PLOS One 11(7): e0158447.

Examples of suitable promoters for directing transcription of the polynucleotide of the present invention in a filamentous fungal host cell are promoters obtained from Aspergillus, Fusarium, Rhizomucor and Trichoderma cells, such as the promoters described in Mukherjee et al., 2013, “Trichoderma: Biology and Applications”, and by Schmoll and Dattenböck, 2016, “Gene Expression Systems in Fungi: Advancements and Applications”, Fungal Biology.

For expression in a yeast host, examples of useful promoters are described by Smolke et al., 2018, “Synthetic Biology: Parts, Devices and Applications” (Chapter 6: Constitutive and Regulated Promoters in Yeast: How to Design and Make Use of Promoters in S. cerevisiae), and by Schmoll and Dattenböck, 2016, “Gene Expression Systems in Fungi: Advancements and Applications”, Fungal Biology.

### Terminators

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells may be obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells may be obtained from Aspergillus or Trichoderma species, such as obtained from the genes for Aspergillus niger glucoamylase, Trichoderma reesei beta-glucosidase, Trichoderma reesei cellobiohydrolase I, and Trichoderma reesei endoglucanase I, such as the terminators described in Mukherjee et al., 2013, “Trichoderma: Biology and Applications”, and by Schmoll and Dattenböck, 2016, “Gene Expression Systems in Fungi: Advancements and Applications”, Fungal Biology.

Preferred terminators for yeast host cells may be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, Yeast 8: 423-488.

mRNA Stabilizers

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, J. Bacteriol. 177: 3465-3471).

Examples of mRNA stabilizer regions for fungal cells are described in Geisberg et al., 2014, Cell 156(4): 812-824, and in Morozov et al., 2006, Eukaryotic Cell 5(11): 1838-1846.

### Leader Sequences

The control sequence may also be a leader, a non-translated region of an mRNA that is important for translation by the host cell. The leader is operably linked to the 5′-terminus of the polynucleotide encoding the polypeptide. Any leader that is functional in the host cell may be used.

Suitable leaders for bacterial host cells are described by Hambraeus et al., 2000, Microbiology 146(12): 3051-3059, and by Kaberdin and Blssi, 2006, FEMS Microbiol. Rev. 30(6): 967-979.

leaders for filamentous fungal host cells may be obtained from the genes for Aspergillus oryzae TAKA amylase and Aspergillus nidulans triose phosphate isomerase.

Suitable leaders for yeast host cells may be obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

### Polyadenylation Sequences

The control sequence may also be a polyadenylation sequence, a sequence operably linked to the 3′-terminus of the polynucleotide which, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

### Signal Peptides

The control sequence, e.g. expression control sequence, may also be a signal peptide coding region that encodes a signal peptide linked to the N-terminus of a polypeptide and directs the polypeptide into the cell's secretory pathway.

Non limiting examples for signal peptides are the signal peptides encoded by SEQ ID NOs: 1 to 247. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is heterologous to the coding sequence. A heterologous signal peptide coding sequence may be required where the coding sequence does not naturally contain a signal peptide coding sequence. Alternatively, a heterologous signal peptide coding sequence may simply replace the natural signal peptide coding sequence to enhance secretion of the polypeptide. Any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Freudl, 2018, Microbial Cell Factories 17: 52.

Effective signal peptide coding sequences for filamentous fungal host cells are the signal peptide coding sequences obtained from the genes for Aspergillus niger neutral amylase, Aspergillus nigerglucoamylase, Aspergillus oryzae TAKA amylase, Humicola insolens cellulase, Humicola insolens endoglucanase V, Humicola lanuginosa lipase, and Rhizomucor miehei aspartic proteinase, such as the signal peptide described by Xu et al., 2018, Biotechnology Letters 40: 949-955.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

### Propeptides

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence. Additionally or alternatively, when both signal peptide and propeptide sequences are present, the polypeptide may comprise only a part of the signal peptide sequence and/or only a part of the propeptide sequence. Alternatively, the final or isolated polypeptide may comprise a mixture of mature polypeptides and polypeptides which comprise, either partly or in full length, a propeptide sequence and/or a signal peptide sequence.

### Regulatory Sequences

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In fungal systems, these regulatory sequences include the dihydrofolate reductase gene that is amplified in the presence of methotrexate, and the metallothionein genes that are amplified with heavy metals.

### Transcription Factors

The control sequence may also be a transcription factor, a polynucleotide encoding a polynucleotide-specific DNA-binding polypeptide that controls the rate of the transcription of genetic information from DNA to mRNA by binding to a specific polynucleotide sequence. The transcription factor may function alone and/or together with one or more other polypeptides or transcription factors in a complex by promoting or blocking the recruitment of RNA polymerase. Transcription factors are characterized by comprising at least one DNA-binding domain which often attaches to a specific DNA sequence adjacent to the genetic elements which are regulated by the transcription factor. The transcription factor may regulate the expression of a protein of interest either directly, i.e., by activating the transcription of the gene encoding the protein of interest by binding to its promoter, or indirectly, i.e., by activating the transcription of a further transcription factor which regulates the transcription of the gene encoding the protein of interest, such as by binding to the promoter of the further transcription factor. Suitable transcription factors for fungal host cells are described in WO 2017/144177. Suitable transcription factors for prokaryotic host cells are described in Seshasayee et al., 2011, Subcellular Biochemistry 52: 7-23, as well in Balleza et al., 2009, FEMS Microbiol. Rev. 33(1): 133-151.

### Expression Vectors

The present invention also relates to recombinant expression vectors comprising a polynucleotide (candidate sequence) obtained by the method of the present invention. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

The vector preferably contains at least one element that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous recombination, such as homology-directed repair (HDR), or non-homologous recombination, such as non-homologous end-joining (NHEJ).

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

More than one copy of a polynucleotide of the present invention may be inserted into a host cell to increase production of a polypeptide. For example, 2 or 3 or 4 or 5 or more copies are inserted into a host cell. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

### Host Cells

The present invention also relates to recombinant host cells, comprising a polynucleotide (candidate biological sequence). The polynucleotide may be operably linked to one or more control sequences that direct the production of a polypeptide of the present invention. Alternatively, when the polynucleotide is a control sequence, the polynucleotide may be operably linked to one or more nucleic acid sequences encoding a polypeptide of interest.

A construct or vector comprising a polynucleotide is introduced into a host cell so that the construct or vector is maintained as a chromosomal integrant or as a self-replicating extra-chromosomal vector as described earlier. The choice of a host cell will to a large extent depend upon the gene encoding the polypeptide and its source. The polypeptide can be native or heterologous to the recombinant host cell. Also, at least one of the one or more control sequences can be heterologous to the polynucleotide encoding the polypeptide. The recombinant host cell may comprise a single copy, or at least two copies, e.g., three, four, five, or more copies of the polynucleotide of the present invention.

The host cell may be any microbial cell useful in the recombinant production of a polypeptide of the present invention, e.g., a prokaryotic cell or a fungal cell.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells. In an embodiment, the Bacillus cell is a Bacillus amyloliquefaciens, Bacillus licheniformis and Bacillus subtilis cell.

For purposes of this invention, Bacillus classes/genera/species shall be defined as described in Patel and Gupta, 2020, Int. J. Syst. Evol. Microbiol. 70: 406-438.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

Methods for introducing DNA into prokaryotic host cells are well-known in the art, and any suitable method can be used including but not limited to protoplast transformation, competent cell transformation, electroporation, conjugation, transduction, with DNA introduced as linearized or as circular polynucleotide. Persons skilled in the art will be readily able to identify a suitable method for introducing DNA into a given prokaryotic cell depending, e.g., on the genus. Methods for introducing DNA into prokaryotic host cells are for example described in Heinze et al., 2018, BMC Microbiology 18:56, Burke et al., 2001, Proc. Natl. Acad. Sci. USA 98: 6289-6294, Choi et al., 2006, J. Microbiol. Methods 64: 391-397, and Donald et al., 2013, J. Bacteriol. 195(11): 2612-2620.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

Fungal cells may be transformed by a process involving protoplast-mediated transformation, Agrobacterium-mediated transformation, electroporation, biolistic method and shock-wave-mediated transformation as reviewed by Li et al., 2017, Microbial Cell Factories 16: 168 and procedures described in EP 238023, Yelton et al., 1984, Proc. Natl. Acad. Sci. USA 81: 1470-1474, Christensen et al., 1988, Bio/Technology 6: 1419-1422, and Lubertozzi and Keasling, 2009, Biotechn. Advances 27: 53-75. However, any method known in the art for introducing DNA into a fungal host cell can be used, and the DNA can be introduced as linearized or as circular polynucleotide.

The fungal host cell may be a yeast cell. “Yeast” as used herein includes ascosporogenous yeast (Endomycetales), basidiosporogenous yeast, and yeast belonging to the Fungi Imperfecti (Blastomycetes). For purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell. In a preferred embodiment, the yeast host cell is a Pichia or Komagataella cell, e.g., a Pichia pastoris cell (Komagataella phaffii).

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell. In a preferred embodiment, the filamentous fungal host cell is an Aspergillus, Trichoderma or Fusarium cell. In a further preferred embodiment, the filamentous fungal host cell is an Aspergillus niger, Aspergillus oryzae, Trichoderma reesei, or Fusarium venenatum cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.

In an aspect, the host cell is isolated.

In another aspect, the host cell is purified.

### Methods of Production

The present invention also relates to methods of producing a polypeptide of interest, comprising (a) cultivating a cell, which produces the polypeptide, under conditions conducive for production of the polypeptide; and optionally, (b) recovering the polypeptide. In one aspect, the cell is a Bacillus cell. In another aspect, the cell is a Bacillus licheniformis cell. In another aspect, the cell is an Aspergillus cell, such as an Aspergillus oryzae, such as an Aspergillus oryzae. In another aspect, the cell is a Trichoderma cell, such as a Trichoderma reesei.

The host cell is cultivated in a nutrient medium suitable for production of the polypeptide using methods known in the art. For example, the cell may be cultivated by shake flask cultivation, or small-scale or large-scale fermentation (including continuous, batch, fed-batch, or solid-state, and/or microcarrier-based fermentations) in laboratory or industrial fermentors in a suitable medium and under conditions allowing the polypeptide to be expressed and/or isolated. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptide, including, but not limited to, the use of specific antibodies, formation of an enzyme product, disappearance of an enzyme substrate, or an assay determining the relative or specific activity of the polypeptide.

The polypeptide may be recovered from the medium using methods known in the art, including, but not limited to, collection, centrifugation, filtration, extraction, spray-drying, evaporation, or precipitation. In one aspect, a whole fermentation broth comprising the polypeptide is recovered. In another aspect, a cell-free fermentation broth comprising the polypeptide is recovered.

The polypeptide may be purified by a variety of procedures known in the art to obtain substantially pure polypeptides and/or polypeptide fragments (see, e.g., Wingfield, 2015, Current Protocols in Protein Science; 80(1): 6.1.1-6.1.35; Labrou, 2014, Protein Downstream Processing, 1129: 3-10).

In an alternative aspect, the polypeptide is not recovered.

### The Candidate Sequence May be Signal Peptide or Encode a Signal Peptide

The present invention also relates to a polynucleotide (candidate sequence) encoding a signal peptide. The polynucleotides may further comprise a gene encoding a protein (polypeptide of interest), which is operably linked to the signal peptide. The protein is preferably heterologous to the signal peptide.

The present invention also relates to nucleic acid constructs, expression vectors and recombinant host cells comprising such polynucleotides.

The present invention also relates to methods of producing a protein, comprising (a) cultivating a recombinant host cell comprising such polynucleotide; and optionally (b) recovering the protein.

The protein may be native or heterologous to a host cell. The term “protein” and “polypeptide of interest” is not meant herein to refer to a specific length of the encoded product and, therefore, encompasses peptides, oligopeptides, and polypeptides. The term “protein” and “polypeptide of interest” also encompasses two or more polypeptides combined to form the encoded product. The proteins also include hybrid polypeptides and fusion polypeptides.

Preferably, the protein is a hormone, enzyme, receptor or portion thereof, antibody or portion thereof, or reporter. For example, the protein may be a hydrolase, isomerase, ligase, lyase, oxidoreductase, or transferase, e.g., an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase.

The gene may be obtained from any prokaryotic, eukaryotic, or other source.

The use of the terms “first”, “second”, “third” and “fourth”, “primary”, “secondary”, “tertiary” etc. does not imply any particular order, but are included to identify individual elements. Moreover, the use of the terms “first”, “second”, “third” and “fourth”, “primary”, “secondary”, “tertiary” etc. does not denote any order or importance, but rather the terms “first”, “second”, “third” and “fourth”, “primary”, “secondary”, “tertiary” etc. are used to distinguish one element from another. Note that the words “first”, “second”, “third” and “fourth”, “primary”, “secondary”, “tertiary” etc. are used here and elsewhere for labelling purposes only and are not intended to denote any specific spatial or temporal ordering. Furthermore, the labelling of a first element does not imply the presence of a second element and vice versa.

It may be appreciated that the Figures comprise some circuitries or operations which are illustrated with a solid line and some circuitries or operations which are illustrated with a dashed line. The circuitries or operations which are comprised in a solid line are circuitries or operations which are comprised in the broadest example embodiment. The circuitries or operations which are comprised in a dashed line are example embodiments which may be comprised in, or a part of, or are further circuitries or operations which may be taken in addition to the circuitries or operations of the solid line example embodiments. It should be appreciated that these operations need not be performed in order presented. Furthermore, it should be appreciated that not all of the operations need to be performed. The exemplary operations may be performed in any order and in any combination.

It is to be noted that the word “comprising” does not necessarily exclude the presence of other elements or steps than those listed.

It is to be noted that the words “a” or “an” preceding an element do not exclude the presence of a plurality of such elements.

It should further be noted that any reference signs do not limit the scope of the claims, that the exemplary embodiments may be implemented at least in part by means of both hardware and software, and that several “means”, “units” or “devices” may be represented by the same item of hardware.

The various exemplary methods, devices, nodes, and systems described herein are described in the general context of method steps or processes, which may be implemented in one aspect by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), etc. Generally, program circuitries may include routines, programs, objects, components, data structures, etc. that perform specified tasks or implement specific abstract data types. Computer-executable instructions, associated data structures, and program circuitries represent examples of program code for executing steps of the methods disclosed herein. The particular sequence of such executable instructions or associated data structures represents examples of corresponding acts for implementing the functions described in such steps or processes.

The present invention is further described by the following examples that should not be construed as limiting the scope of the invention.

### EXAMPLES

### Molecular Biological Methods

DNA manipulations and transformations were performed by standard molecular biology methods as described in:
Sambrook et al. (1989): Molecular cloning: A laboratory manual. Cold Spring Harbor laboratory, Cold Spring Harbor, NY.
Ausubel et al. (eds) (1995): Current protocols in Molecular Biology. John Wiley and Sons.
Harwood and Cutting (eds) (1990): Molecular Biological Methods for Bacillus. John Wiley and Sons.

Enzymes for DNA manipulation were obtained from New England Biolabs, Inc. and used essentially as recommended by the supplier.

Direct transformation into B. licheniformis was one as previously described in patent US 2019/0185847 A1. Conjugation into B. licheniformis was performed as described in WO 2018/077796 A1.

Genomic DNA was prepared by using the commercially available QIAamp DNA Blood Kit from Qiagen. The respective DNA fragments were amplified by PCR using the Phusion Hot Start DNA Polymerase system (Thermo Scientific). PCR amplification reaction mixtures contained 1 μL (0.1 pg) of template DNA, 1 μL of sense primer (20 pmol/μL), 1 μL of anti-sense primer (20 pmol/μL), 10 μL of 5×PCR buffer with 7.5 mM MgCl₂, 8 μL of dNTP mix (1.25 mM each), 39 μL water, and 0.5 μL (2 U/) DNA polymerase. A thermocycler was used to amplify the fragment. The PCR products were purified from a 1.2% agarose gel with 1×TBE buffer using the Qiagen QIAquick Gel Extraction Kit (Qiagen, Inc., Valencia, CA) according to the manufacturer's instructions.

The condition for POE-PCR is as follows: purified PCR products were used in a subsequent PCR reaction to create a single fragment using splice overlapping PCR (SOE) using the Phusion Hot Start DNA Polymerase system (Thermo Scientific) as follows. The very 5′ end fragment and the very 3′ end fragment have complementary end which will allow the SOE to concatemer into the POE PCR product. The PCR amplification reaction mixture contained 50 ng of each of the three gel purified PCR products. POE PCR was performed as described in (You, C et al (2017) Methods Mol. Biol. 116, 183-92).

### Media

Bacillus strains were grown on LB agar (10 g/L Tryptone, 5 g/L yeast extract, 5 g/L NaCl, 15 g/L agar) plates or in TY liquid medium (20 g/L Tryptone, 5 g/L yeast extract, 7 mg/L FeCl₂, 1 mg/L MnCl₂, 15 mg/L MgCl₂). To select for erythromycin resistance, agar and liquid media were supplemented with 5 μg/ml erythromycin.

LB agar: 10 g/l peptone from casein; 5 g/l yeast extract, 10 g/l sodium chloride; 12 g/l Bacto-agar adjusted to pH 7.0+/−0.2. Premix from Merck was used (LB-agar (Miller) 110283)

### Fermentation

Strains were fermented in microtiter plates with nutrient controlled media at 37 °C, 1000 rpm.

### Protease Assay

The serine endopeptidase hydrolyses the substrate N-Succinyl-Ala-Ala-Pro-Phe p-nitroanilide. The reaction was performed at Room Temperature at pH 9.0. The release of pNA results in an increase of absorbance at 405 nm and this increase is proportional to the enzymatic activity measured against a standard.

### Strains

| | |
|---|---|
| MOL3320: | Bacillus licheniformis, amyL, aprL, bglC, cypX, |
| | forD, gntP, lacA2, mprL, sacB, spoIIAC, xylA, |
| | ara::Mad7d-sgRNA::mecA-ERM (as described in |
| | patent US 2019/0185847 A1) |

### Example 1: Generation of Artificial Signal Peptides and Codon Variants Encoding the Same

Using the GAN of the invention, the Alkalihalobacillus clausii protease (SEQ ID NO: 292) was used as input biological sequence to first generate two artificial SP amino acid sequences (SEQ ID NO: 280, and SEQ ID NO: 279), and then subsequently generate codon variants encoding the two artificial signal peptides. SEQ ID NOs: 229-235 show generated codon variants encoding the SP of SEQ ID NO: 280. SEQ ID NOs: 222-228, and SEQ ID NO: 237 show generated codon variants encoding the SP of SEQ ID NO: 279 and 282.

Synthetic DNA was ordered to contain the protease expression cassette under control of the triple promoter (as described in WO 99/43835) and fused to a polynucleotide sequence encoding one of the generated signal peptides. The protease expression cassette was combined with an upstream ara flanking region including the triple promoter and a downstream flanking region of the ara locus, including the ERM selection marker, in a POE PCR (patent US 2019/0185847 A1). The generated material was used for transformation into MOL3320 as described in patent US 2019/0185847 A1. Selection was done on ERM.

The performance of any given candidate biological sequence (signal peptide) is evaluated in the laboratory by ligating the candidate biological sequence in front of a nucleotide sequence encoding the protease.

Two rounds of generations were applied:
1) In the first round, a generative model is applied to input biological sequences=protease amino acid sequence of SEQ ID NO: 292 (in the following we refer to this set as X) to obtain a plurality of candidate biological sequences=signal peptide amino acid sequences SEQ ID NOs: 280, 279, and 282 (in the following we refer to this set as Y).
2) In the second round, a generative model is applied to the signal peptide amino acid sequences of set Y (such that these are themselves input biological sequences in this second round) to obtain a plurality of candidate biological sequences=DNA sequences encoding the signal peptides (in the following we refer to this set as Z, Z is consisting of SEQ ID NOs: 222-235 and SEQ ID NO: 237).

In the first round, the sequence of set X is an input biological sequence and the sequences of set Y are candidate biological sequences. In the second round, the sequences of set Y are input biological sequences and the sequences of set Z are candidate biological sequences. The sequence of set X is a polypeptide, the sequences of set Y are polypeptides (signal peptides), and the sequences of set Z are polynucleotide sequences encoding signal peptides.

In this example, in the first round, the candidate biological sequences of set Y are a plurality of signal peptide sequences compatible with the input biological sequence of set X. In the second round, the candidate biological sequences of set Z are polynucleotide sequences encoding signal peptides and the input biological sequences of set Y are polypeptides in the form of signal peptides.

In the first round, the input amino acid sequence of set X is a mature peptide (i.e., mature protease) and the candidate biological sequences of set Y are a plurality of signal peptide amino acid sequences (polypeptides) compatible with the mature peptides, in such a way that the mature peptide is paired with a plurality of signal peptides. In the second round, the input biological sequences of set Y are signal peptide amino acid sequences (polypeptides), and the candidate biological sequences of set Z are the corresponding codon-optimized DNA sequences. The candidate biological sequences are a plurality of codon-optimized DNA sequences obtained by applying a generative model (in this example, a GAN) to the input signal peptide amino acid sequences. Alternatively, the input biological sequence is an entire protein amino acid sequence (protease), and the candidate biological sequences are corresponding codon-optimized DNA sequences.

In the first round, signal peptide amino acid sequences are generated corresponding to the input mature peptides and then, in the second round, the generated signal peptides are codon encoded.

In this example, two rounds have been applied sequentially from sequences of sets X to Y, and of sets Y to Z. Note that in a more general case going from set X to set Y does not need to be followed by going from set Y to set Z. Alternatively or additionally, it is possible to directly start at set Y to directly obtain a set Z.

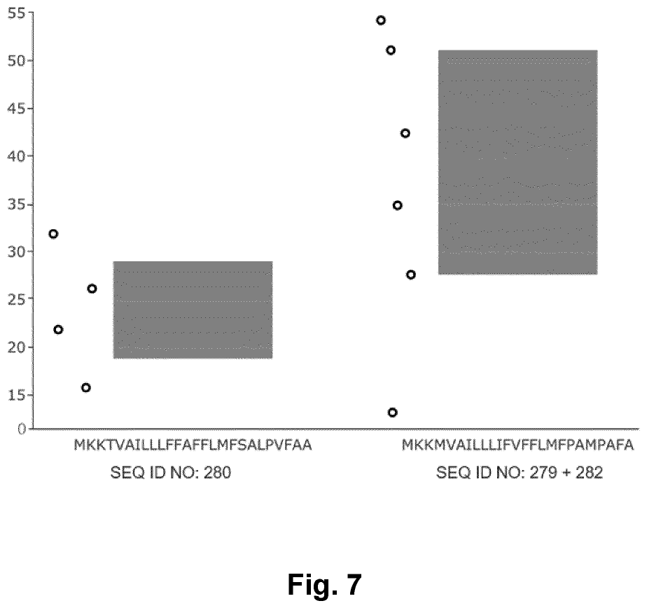

To evaluate the effect of the artificial signal peptides (candidate biological sequences) on the activity of the protease, strains were fermented for app. 120 hours and protease activity was measured at the end of fermentation. The performance of the resulting candidate biological sequences was evaluated. As can be seen from FIG. 7, the GAN of the invention successfully generated artificial signal peptides and codon variants which can be utilized for protease expression. Furthermore, the GAN of the invention allowed the design of several codon-variants (shown in circles) which allowed fine-tuned protease expression, the codon variants showing up to 50-fold differential protease activities.

Although some of the codon variants of the two artificial signal peptides show similar protease activities, FIG. 7 also reveals that with the model of the invention several superior codon variants are generated, which result in significantly increased protease activities. Depending on the desired protease activity, a suitable codon variant can be chosen without changing the actual amino acid sequence of the signal peptide.

### Example 2: Generation of Artificial Codon Variants for Wild-Type Signal Peptides

Using different wild-type signal peptide amino acid sequences as input biological sequences (SEQ ID NOs: 248, 258, 269, 265, 266, 278, 249, 264, 257, 255, 276, 252, 256, 253, and 274) a plurality of artificial DNA sequences with different codons was obtained as candidate biological sequences, without changing the signal peptide's amino acid sequence.

FIG. 8 shows box plots of protease activities for the different signal peptides and artificial codon variants (circles). FIG. 8 also includes the artificial signal peptides of SEQ ID NOs: 279, 280 and 282 generated in Example 1.

Signal Peptides and their Coding Sequences as Shown in FIG. 8:
SEQ ID NO: 248 (encoded by codon variants with SEQ ID NOs: 1-10),
SEQ ID NO: 249 (encoded by codon variants with SEQ ID NOs: 11-15),
SEQ ID NO: 252 (encoded by codon variants with SEQ ID NOs: 32-41),
SEQ ID NO: 253 (encoded by codon variants with SEQ ID NOs: 42-52),
SEQ ID NO: 255 (encoded by codon variants with SEQ ID NOs: 60-67),
SEQ ID NO: 256 (encoded by codon variants with SEQ ID NOs: 68-74),
SEQ ID NO: 257 (encoded by codon variants with SEQ ID NOs: 75-84),
SEQ ID NO: 258 (encoded by codon variants with SEQ ID NOs: 85-98),
SEQ ID NO: 264 (encoded by codon variants with SEQ ID NOs: 116-129),
SEQ ID NO: 265 (encoded by codon variants with SEQ ID NOs: 130-137),
SEQ ID NO: 266 (encoded by codon variants with SEQ ID NOs: 138-147),
SEQ ID NO: 269 (encoded by codon variants with SEQ ID NOs: 162-167),
SEQ ID NO: 274 (encoded by codon variants with SEQ ID NOs: 184-193),
SEQ ID NO: 276 (encoded by codon variants with SEQ ID NOs: 196-209),
SEQ ID NO: 277 (encoded by codon variants with SEQ ID NOs: 210-216),
SEQ ID NO: 278 (encoded by codon variants with SEQ ID NOs: 217-221),
SEQ ID NOs: 279 and 282 (encoded by codon variants with SEQ ID NOs: 222-228, and 237), and
SEQ ID NO: 280 (encoded by codon variants with SEQ ID NOs: 229-235).

The target performance was measured using a protease activity assay, which is a proxy for yield (Y-axis). For every signal peptide, any impact on the protease activity assay can be attributed to the candidate biological sequence, i.e., the artificial DNA sequence encoding the signal peptide. Thus, the activity assay becomes a proxy for the performance of the chosen codon variant. The bar next to the circles indicates the distribution of values, using a box plot.

FIG. 8 shows that artificial signal peptide codon variants can cause an up to 100-fold change of protease activity, either within one single signal peptide amino acid sequence (see e.g., high variety of protease activities for the artificial codon variants of SEQ ID NO: 248 on very top of FIG. 8) or across different signal peptide amino acid sequences encoded by different codon variants. The method of the invention can thus be utilized to generate artificial, and with regards to protein expression superior, codon-variants for already existing wild-type signal peptides. Furthermore, FIG. 8 confirms that the artificial signal peptide amino acid sequences (SEQ ID NO: 279, 282 and 280) and corresponding codon variants generated in Example 1 result in protease expression which is similar or superior to some of the amino acid sequences of wild-type signal peptides.

### Example 3: Validating the Artificial Codon Variants

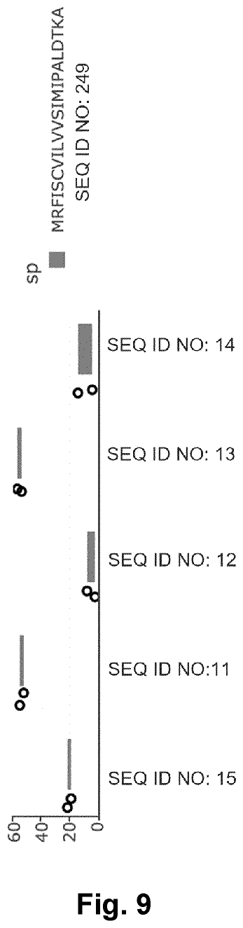

A single wild-type signal peptide amino acid sequence (SEQ ID NO: 249) encoded by five artificial codon variants generated in Example 2 was further assessed. The five codon variants (SEQ ID NOs: 11-15) were analyzed for protease activities (FIG. 9). Protease activities (Y-axis) were measured in replicates for each codon sequence (X-axis). As can be seen from FIG. 9, there is a significant difference in protease activity depending on the exact codon sequence. Since each artificial codon sequence encodes the same signal peptide amino acid sequence, this plot clearly shows the potential of being capable of varying the codon encoding of a signal peptide. Further, FIG. 9 shows that depending on the codon variant, protease activities can be increased between ca. 2- to 10-fold.

### Example 4: Comparison of Artificial SP Codon Variants Against the aprL Signal Peptide

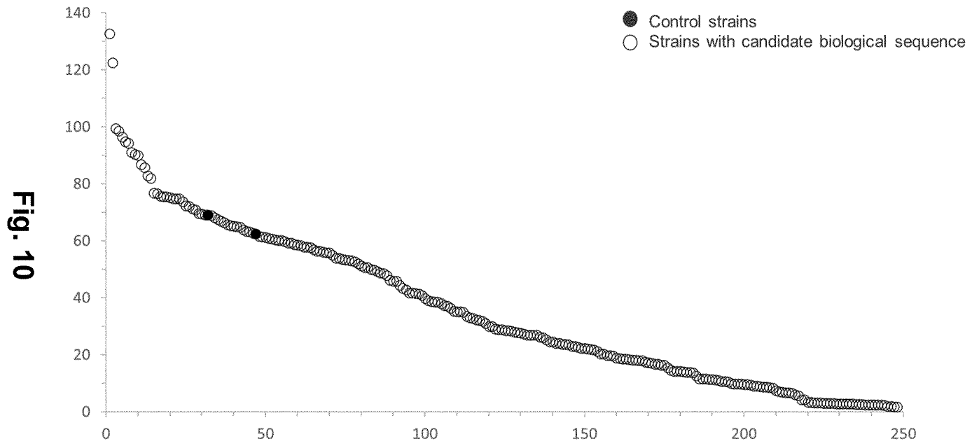

As shown in the previous examples, applying the GAN of the invention with either an Alkalihalobacillus clausii protease or a wild-type signal peptide as input biological sequences, artificial signal peptides and artificial signal peptide-encoding polynucleotides can be generated, respectively. FIG. 10 compares the protease activities of artificial codon variants against protease activities of the wild-type aprL signal peptide coding sequence (black dots=control strains with aprL SP). A total of 246 (X-axis) different signal peptide-encoding polynucleotides (SEQ ID NOs: 1-246) were generated with the GAN of the invention and according to the methods disclosed in Examples 1 and 2. These polynucleotides encode a total of 42 signal peptide amino acid sequences (SEQ ID NOs: 248-289). The aprL signal peptide (SEQ ID NO: 290) encoded by the polynucleotide with SEQ ID NO: 247 was used as control signal peptide.

To evaluate the effect of the signal peptide coding sequences (candidate biological sequences) on the activity of the protease, strains were fermented for app. 120 hours and protease activity was measured at the end of fermentation. The codon variants and aprL control were then ranked based on their protease activities. A subset of about 50 of the 246 generated codon variants showed a protease activity which is similar or significantly improvement compared to control strains with the aprL signal peptide (FIG. 10). Notably, the two artificial codon variants with the highest protease activities show approximately twice the protease activity of the aprL SP controls. These results show that the model of the invention successfully can generate signal peptides and/or codon variants that outperform natural signal peptides in terms of protein expression.

Although features have been shown and described, it will be understood that they are not intended to limit the claimed disclosure, and it will be made obvious to those skilled in the art that various changes and modifications may be made without departing from the scope of the claimed disclosure. The specification and drawings are, accordingly, to be regarded in an illustrative rather than restrictive sense. The claimed disclosure is intended to cover all alternatives, modifications, and equivalents. Any equivalent aspects are intended to be within the scope of this disclosure. Indeed, various modifications of the invention in addition to those shown and described herein will become apparent to those skilled in the art from the description. Such modifications are also intended to fall within the scope of the appended claims. In the case of conflict, the present disclosure including definitions will control.

1. A method, performed in an electronic device, for providing a candidate biological sequence, the method comprising:
obtaining input data indicative of an input biological sequence;
determining the candidate biological sequence by applying a generative model to the input data, wherein the generative model is non-unidirectional; and
providing biological sequence data indicative of the candidate biological sequence,
wherein the candidate biological sequence is increasing compatibility with a host cell.

2. The method according to claim 1, wherein the input biological sequence is one or more of: an amino acid sequence of a polypeptide of interest, a nucleic acid sequence encoding a polypeptide of interest, a control sequence, e.g., an expression control sequence, and a nucleic acid sequence encoding a control sequence, e.g., an expression control sequence.

3. The method according to any of the previous claims, wherein the candidate biological sequence is one or more of: a control sequence, e.g., an expression control sequence, a nucleic acid sequence encoding a control sequence, e.g., an expression control sequence, an amino acid sequence of a polypeptide of interest, and a nucleic acid sequence encoding a polypeptide of interest.

4. The method according to any of the previous claims, wherein the input biological sequence is an amino acid sequence of a polypeptide of interest and/or a nucleic acid sequence encoding a polypeptide of interest, and wherein the candidate biological sequence is a nucleic acid sequence.

5. The method according to any of the previous claims, wherein the input biological sequence is an amino acid sequence of a polypeptide of interest and/or a nucleic acid sequence encoding a polypeptide of interest, and wherein the candidate biological sequence is a control sequence, e.g., an expression control sequence, and/or a nucleic acid sequence encoding a control sequence, e.g., an expression control sequence.

6. The method according to any of the previous claims, wherein the input biological sequence is a control sequence, e.g., an expression control sequence, and/or a nucleic acid sequence encoding a control sequence, e.g., an expression control sequence, and wherein the candidate biological sequence is a nucleic acid sequence encoding a polypeptide of interest.

7. The method according to any of the previous claims, wherein the generative model is one or more of: a generative adversarial network model, a Wasserstein generative adversarial network model, a diffusion model, and a variational autoencoder.

8. The method according to any of the previous claims, wherein applying the generative non-unidirectional model to the input data comprises partitioning the generative non-unidirectional model into a plurality of generators, wherein each generator of the plurality of generators is configured to determine, based on the input data, one or more candidate biological sequences for a subset of nucleotides and/or a subset of amino acids and a predetermined criterion.

9. The method according to claim 8, wherein determining the candidate biological sequence by applying the generative model to the input data comprises:
predicting, using the generator, the compatibility of the candidate biological sequence with the host cell; and
determining the candidate biological sequence having a predicted compatibility meeting the predetermined criterion.

10. The method according to any of claims 8-9, wherein the predetermined criterion is based on one or more of:
a proportion of the set of nucleotides in the candidate biological sequence;
a class of host cell;
a host cell genus or species;
a GC content of a host cell genome;
a GC content of the candidate biological sequence; and
a parameter associated with a property of the candidate biological sequence.

11. The method according to any of the previous claims, the method comprising training the generative model based on a training set of biological sequences, wherein the training set of biological sequences includes training data indicative of one or more biological sequences related to the host cell.

12. The method according to claim 11, wherein at least a subset of the training set of biological sequences is heterologous to the genus of the host cell, preferably heterologous to one or more species of the host cell.

13. The method according to any of claims 11-12, wherein the training data comprises training input data indicative of one or more of: an amino acid sequence of a polypeptide of interest, a nucleic acid sequence encoding a polypeptide of interest, a control sequence, e.g., an expression control sequence, and a nucleic acid sequence encoding a control sequence, e.g., an expression control sequence.

14. The method according to any of claims 11-13, wherein the training data comprises training output data indicative of one or more of: a control sequence, e.g., an expression control sequence, a nucleic acid sequence encoding a control sequence, e.g., an expression control sequence, an amino acid sequence of a polypeptide of interest, and a nucleic acid sequence encoding a polypeptide of interest.

15. The method according to any of claims 11-14, wherein training the generative model comprises predicting, using a discriminator taking as input the training set of biological sequences, and a training candidate biological sequence, a score indicative of the training candidate biological sequence being a referenced biological sequence.

16. The method according to any of the previous claims, the method comprising obtaining, from a test environment data repository, experimental data associated with the candidate biological sequence and the host cell; wherein the experimental data indicates a compatibility, e.g., a yield performance, of the candidate biological sequence associated with the host cell.

17. The method according to claim 16, the method comprising validating the candidate biological sequence based on the experimental data.

18. The method according to any of claims 16-17, the method comprising selecting one or more generators based on the experimental data.

19. The method according to any of claims 16-18, the method comprising adapting the generative model based on the experimental data.

20. The method according to any of the previous claims, wherein obtaining input data indicative of an input biological sequence comprises obtaining the input data for the input biological sequence from a database and/or a memory of the electronic device.

21. An electronic device comprising a memory circuitry, a processor circuitry, and an interface, wherein the electronic device is configured to perform any of the methods according to any of claims 1-20.

22. A computer readable storage medium storing one or more programs, the one or more programs comprising instructions, which when executed by an electronic device cause the electronic device to perform any of the methods of claims 1-20.

23. A recombinant host cell comprising in its genome a first polynucleotide encoding a control sequence, e.g., an expression control sequence, and a second polynucleotide operably linked to the first polynucleotide encoding a polypeptide of interest, wherein the first or second polynucleotide is the candidate biological sequence obtained by the method from any one of claims 1-20.


SEQUENCE LISTING (292 sequences)

SEQ ID NO: 1 - DNA, 81 bp, synthetic construct
  source 1..81: mol_type = other DNA
atgaagaaggtaatgacctccgtgttggttggtgctattgtcttcacgggcgccggtgttacagctgtccctgttgaggcg

SEQ ID NO: 2 - DNA, 81 bp, synthetic construct
  source 1..81: mol_type = other DNA
atgaagaaggtaatgacctccgtgttggttggagctatagtcttcacaggcgcgggtgtcacagctgtccctgttgaagcg

SEQ ID NO: 3 - DNA, 81 bp, synthetic construct
  source 1..81: mol_type = other DNA
atgaaaaaggtaatgacaagtgtgttagttggtgctattgtcttcacaggcgcgggtgtcacagctgtccctgttgaggcg

SEQ ID NO: 4 - DNA, 81 bp, synthetic construct
  source 1..81: mol_type = other DNA
atgaagaaggtaatgacgtcagtgttagttggagccattgtcttcacgggcgccggtgtcacagctgtccctgttgaagcg

SEQ ID NO: 5 - DNA, 81 bp, synthetic construct
  source 1..81: mol_type = other DNA
atgaaaaaagtaatgacatccgtattagttggagctattgtctttacaggcgctggtgttacagctgtccctgttgaagcg

SEQ ID NO: 6 - DNA, 81 bp, synthetic construct
  source 1..81: mol_type = other DNA
atgaaaaaggtaatgacgtcagtgttagttggagctattgtctttacaggcgcgggtgtcacagctgtccctgttgaagcg

SEQ ID NO: 7 - DNA, 81 bp, synthetic construct
  source 1..81: mol_type = other DNA
atgaaaaaggtaatgacaagtgtattagttggagctattgtctttacaggagctggtgttacagctgtccctgttgaagcg

SEQ ID NO: 8 - DNA, 81 bp, synthetic construct
  source 1..81: mol_type = other DNA
atgaagaaggtaatgacgtcagtgttggttggtgctatagtttttacaggcgcgggtgtgacagctgtccctgttgaagcg

SEQ ID NO: 9 - DNA, 81 bp, synthetic construct
  source 1..81: mol_type = other DNA
atgaaaaaggtaatgacgtccgtgttggttggagctattgttttcacaggcgccggtgttacagctgtccctgttgaagcg

SEQ ID NO: 10 - DNA, 81 bp, synthetic construct
  source 1..81: mol_type = other DNA
atgaagaaggtaatgacgtcagtgttagttggtgcaattgtctttacaggcgcgggtgtgacagctgtccctgttgaagcg

SEQ ID NO: 11 - DNA, 66 bp, synthetic construct
  source 1..66: mol_type = other DNA
atgcgattcattagttgcgttatattggtggtaagtatcatgatacctgcacttgatacaaaagcg

SEQ ID NO: 12 - DNA, 66 bp, synthetic construct
  source 1..66: mol_type = other DNA
atgcgcttcattagttgcgttatattggttgtaagtatcatgatacctgcccttgatacgaaagcg

SEQ ID NO: 13 - DNA, 66 bp, synthetic construct
  source 1..66: mol_type = other DNA
atgagatttattagttgcgttatattagtagtaagtatcatgatccctgcacttgacacaaaagcg

SEQ ID NO: 14 - DNA, 66 bp, synthetic construct
  source 1..66: mol_type = other DNA
atgcgcttcattagttgcgttatattggtcgtaagtatcatgatacctgcccttgataccaaagcg

SEQ ID NO: 15 - DNA, 66 bp, synthetic construct
  source 1..66: mol_type = other DNA
atgcgcttcattagttgcgttattttggttgtaagtatcatgattccggcccttgataccaaagcg

SEQ ID NO: 16 - DNA, 87 bp, synthetic construct
  source 1..87: mol_type = other DNA
atgcgcaaagtggtcacctattctttggcagccgcaatcggcctgtctggactcataggattcgctggtgtcggatcggtatctgcg

SEQ ID NO: 17 - DNA, 87 bp, synthetic construct
  source 1..87: mol_type = other DNA
atgcgcaaagtagtcacctattccttggcagccgcaatcggcctgtctggactcatcggattcgctggtgtcggatcggtatctgcg

SEQ ID NO: 18 - DNA, 87 bp, synthetic construct
  source 1..87: mol_type = other DNA
atgcgaaaagtagttacgtattcattggcagctgcaattggactgtcaggacttatcggattcgctggtgttggatcggtatctgcg

SEQ ID NO: 19 - DNA, 87 bp, synthetic construct
  source 1..87: mol_type = other DNA
atgaggaaagtagtcacctattcattggcagctgcaatcggactgtcgggacttataggtttcgctggtgttggatcggtatctgcg

SEQ ID NO: 20 - DNA, 87 bp, synthetic construct
  source 1..87: mol_type = other DNA
atgcgaaaagtagtcacctattctttggcagccgcaattggactgtctggacttatcggattcgctggtgtcggatcggtctctgcg

SEQ ID NO: 21 - DNA, 87 bp, synthetic construct
  source 1..87: mol_type = other DNA
atgcgcaaagtggtcacctattccttggcagccgcaatcggcctgtcgggactcatcggcttcgctggtgtcgggtcggtctcagcg

SEQ ID NO: 22 - DNA, 87 bp, synthetic construct
  source 1..87: mol_type = other DNA
atgcgaaaagtagtcacgtattcattggcagctgcaatcggactgtcgggacttataggattcgctggtgttggatcggtatcagcg

SEQ ID NO: 23 - DNA, 87 bp, synthetic construct
  source 1..87: mol_type = other DNA
atgcgtaaagtagtcacctattccttggcagccgcaatcggactgtcaggacttatcggattcgctggtgttggatcggtatcagcg

SEQ ID NO: 24 - DNA, 87 bp, synthetic construct
  source 1..87: mol_type = other DNA
atgaggaaagtagtcacgtattcattggcagctgcaatcggactgtcgggacttatcggatttgctggtgttggatcggtatcagcg

SEQ ID NO: 25 - DNA, 87 bp, synthetic construct
  source 1..87: mol_type = other DNA
atgcgtaaagtagttacctattctttggcagctgcaatcggcctgtctggacttatcggatttgctggtgtcggatcggtatctgcg

SEQ ID NO: 26 - DNA, 87 bp, synthetic construct
  source 1..87: mol_type = other DNA
atgaggaaagtagttacctattctttggcagccgcaatcggcctgtctggacttatcggattcgctggtgttggatcggtatctgcg

SEQ ID NO: 27 - DNA, 99 bp, synthetic construct
  source 1..99: mol_type = other DNA
atgttaaaaaaagcaaaaggaaaagttattgcaggaacattagttgtaggccttgttgctggaggaggagcggttttgggagcaacagacgccggagcg

SEQ ID NO: 28 - DNA, 99 bp, synthetic construct
  source 1..99: mol_type = other DNA
atgttaaaaaaagcaaagggaaaagttattgcaggaacactggttgtaggccttgttgctggaggaggagcggttttgggagcaacagacgcgggagcg

SEQ ID NO: 29 - DNA, 99 bp, synthetic construct
  source 1..99: mol_type = other DNA
atgctgaaaaaagcaaagggaaaagttattgcaggaacattagttgtaggccttgttgcgggaggaggagcggttttgggcgcaacagacgccggtgcg

SEQ ID NO: 30 - DNA, 99 bp, synthetic construct
  source 1..99: mol_type = other DNA
atgcttaaaaaagcaaagggaaaagttatcgcagggacattagttgtaggccttgttgcgggaggaggagcagtcttgggcgcaacggatgccggagcg

SEQ ID NO: 31 - DNA, 99 bp, synthetic construct
  source 1..99: mol_type = other DNA
atgcttaaaaaagcaaagggaaaagttatcgcgggaacacttgttgtaggccttgtcgcgggaggaggagcggttttgggcgcaacagacgcgggagcg

SEQ ID NO: 32 - DNA, 75 bp, synthetic construct
  source 1..75: mol_type = other DNA
atgaaaacaaaacagacaattgctgcgagtacactagcgcttgcgatgattgctgtagcgaatagtgcgcacgcg

SEQ ID NO: 33 - DNA, 75 bp, synthetic construct
  source 1..75: mol_type = other DNA
atgaagacaaaacagacaattgctgcatcgacactagcgttagcaatgattgctgtagcgaatagtgcacatgcg

SEQ ID NO: 34 - DNA, 75 bp, synthetic construct
  source 1..75: mol_type = other DNA
atgaagacaaaacagacaattgctgcatcgacgctagcgcttgcaatgattgctgtggcgaatagtgcacacgcg

SEQ ID NO: 35 - DNA, 75 bp, synthetic construct
  source 1..75: mol_type = other DNA
atgaaaacaaaacagacaattgctgcatcgacgctagctcttgcaatgattgctgtggcgaatagtgcacacgcg

SEQ ID NO: 36 - DNA, 75 bp, synthetic construct
  source 1..75: mol_type = other DNA
atgaaaacaaaacagacaattgctgcatcgacgctagctcttgcaatgattgctgttgcgaattctgcgcacgcg

SEQ ID NO: 37 - DNA, 75 bp, synthetic construct
  source 1..75: mol_type = other DNA
atgaaaacaaaacagacaattgctgcatctacgctcgctctagcaatgattgctgtcgcgaattctgcacacgcg

SEQ ID NO: 38 - DNA, 75 bp, synthetic construct
  source 1..75: mol_type = other DNA
atgaaaacaaaacagaccattgcggcgtctacgctagctctagcgatgattgctgtcgcgaattctgcgcacgcg

SEQ ID NO: 39 - DNA, 75 bp, synthetic construct
  source 1..75: mol_type = other DNA
atgaaaacaaaacagacaattgctgcatcgacactagctctagcaatgattgcggtagcgaacagtgcacacgcg

SEQ ID NO: 40 - DNA, 75 bp, synthetic construct
  source 1..75: mol_type = other DNA
atgaaaacaaaacagacaattgctgcatctacgctagctctagcaatgattgctgtagcgaattctgcacatgcg

SEQ ID NO: 41 - DNA, 75 bp, synthetic construct
  source 1..75: mol_type = other DNA
atgaagacaaaacagacaatagctgcatcgacgctagctctagcaatgattgctgtagcgaacagtgcgcacgcg

SEQ ID NO: 42 - DNA, 60 bp, synthetic construct
  source 1..60: mol_type = other DNA
atgaaaaaacaaatcgcaattcttgcagtaagtgttgcattcttcatcggggtctgggcg

SEQ ID NO: 43 - DNA, 60 bp, synthetic construct
  source 1..60: mol_type = other DNA
atgaaaaaacaaattgccattcttgctgtatctgttgcattcttcattggcgtctgggcg

SEQ ID NO: 44 - DNA, 60 bp, synthetic construct
  source 1..60: mol_type = other DNA
atgaaaaaacaaatagcaatattggctgtaagtgtggcattcttcattggcgtatgggcg

SEQ ID NO: 45 - DNA, 60 bp, synthetic construct
  source 1..60: mol_type = other DNA
atgaaaaaacaaattgcaattctggcggtaagtgttgcattcttcatcggagtctgggcg

SEQ ID NO: 46 - DNA, 60 bp, synthetic construct
  source 1..60: mol_type = other DNA
atgaaaaaacaaattgcaatattggctgtaagtgttgcattcttcataggagtctgggcg

SEQ ID NO: 47 - DNA, 60 bp, synthetic construct
  source 1..60: mol_type = other DNA
atgaaaaaacaaatagcaattcttgctgtaagtgttgcattcttcatcggggtctgggcg

SEQ ID NO: 48 - DNA, 60 bp, synthetic construct
  source 1..60: mol_type = other DNA
atgaaaaaacaaatagccatcctggcggtaagtgttgcattcttcatcggcgtctgggcg

SEQ ID NO: 49 - DNA, 60 bp, synthetic construct
  source 1..60: mol_type = other DNA
atgaaaaaacaaatagcaattttggctgtaagtgttgcattcttcatcggggtctgggcg

SEQ ID NO: 50 - DNA, 60 bp, synthetic construct
  source 1..60: mol_type = other DNA
atgaaaaaacaaatagccattctggcggtaagtgttgcattcttcatcggcgtctgggcg

SEQ ID NO: 51 - DNA, 60 bp, synthetic construct
  source 1..60: mol_type = other DNA
atgaagaaacaaatagcaatattggcggtaagtgtagcattcttcataggcgtctgggcg

SEQ ID NO: 52 - DNA, 60 bp, synthetic construct
  source 1..60: mol_type = other DNA
atgaaaaaacaaatagcaattttagctgtaagtgttgcattcttcataggggtctgggcg

SEQ ID NO: 53 - DNA, 75 bp, synthetic construct
  source 1..75: mol_type = other DNA
atgaagaaagtacagtgctttatactcatggcggtcatattatgcagttcaatgtggtcagcaacggtcaaggcg

SEQ ID NO: 54 - DNA, 75 bp, synthetic construct
  source 1..75: mol_type = other DNA
atgaaaaaagtacagtgttttatacttatggcggttatattatgcagttcaatgtggtcagcaacggtaaaggcg

SEQ ID NO: 55 - DNA, 75 bp, synthetic construct
  source 1..75: mol_type = other DNA
atgaagaaagtacaatgtttcatattaatggctgtcatattatgtagttcaatgtggtcagcaacagtcaaggcg

SEQ ID NO: 56 - DNA, 75 bp, synthetic construct
  source 1..75: mol_type = other DNA
atgaagaaagtacagtgttttatattaatggcggtcatattatgcagttcaatgtggtcagcaacggtcaaggcg

SEQ ID NO: 57 - DNA, 75 bp, synthetic construct
  source 1..75: mol_type = other DNA
atgaagaaagtgcagtgttttatactaatggcggtcatattatgtagttcaatgtggtcagcaacagtcaaggcg

SEQ ID NO: 58 - DNA, 75 bp, synthetic construct
  source 1..75: mol_type = other DNA
atgaagaaagtacagtgttttatattaatggctgtcatattatgtagttcaatgtggtcagcaacagtcaaggcg

SEQ ID NO: 59 - DNA, 75 bp, synthetic construct
  source 1..75: mol_type = other DNA
atgaagaaagtacagtgttttatactaatggctgtcatattatgtagttcaatgtggtcagcaactgtcaaggcg

SEQ ID NO: 60 - DNA, 81 bp, synthetic construct
  source 1..81: mol_type = other DNA
atgaaaagaacactagtaatgactaccaccctaatcataacaatcttaatcgtctccctttttttacggtttggctctgcg

SEQ ID NO: 61 - DNA, 81 bp, synthetic construct
  source 1..81: mol_type = other DNA
atgaaaagaacgctagtaatgactaccaccttaatcatcacgatattaatcgtatccttatttttacggtttgggtctgcg

SEQ ID NO: 62 - DNA, 81 bp, synthetic construct
  source 1..81: mol_type = other DNA
atgaaaagaacactagtaatgactaccacattaatcattacaattttaatcgtatctttatttttaaggtttggatctgcg

SEQ ID NO: 63 - DNA, 81 bp, synthetic construct
  source 1..81: mol_type = other DNA
atgaaaagaacactagtaatgactaccacattaataataacaatattaatcgtatcattatttttaaggtttggatctgcg

SEQ ID NO: 64 - DNA, 81 bp, synthetic construct
  source 1..81: mol_type = other DNA
atgaaaagaacactagtaatgactaccacattaattattacaatattaatcgtctctttatttttaaggtttggatctgcg

SEQ ID NO: 65 - DNA, 81 bp, synthetic construct
  source 1..81: mol_type = other DNA
atgaaaagaacactagtaatgactaccacattaatcattacaatcttaatcgtatctttatttttaagatttggatctgcg

SEQ ID NO: 66 - DNA, 81 bp, synthetic construct
  source 1..81: mol_type = other DNA
atgaaaagaacactagtaatgactaccaccctaatcattacaattttaatcgtctccttatttctgaggtttggatctgcg

SEQ ID NO: 67 - DNA, 81 bp, synthetic construct
  source 1..81: mol_type = other DNA
atgaaaagaacactagtaatgactaccacactaattattacaattttaatcgtatcactttttttaaggtttggatcagcg

SEQ ID NO: 68 - DNA, 75 bp, synthetic construct
  source 1..75: mol_type = other DNA
atgaaacgtttcgtatgctttttgtttagtcttgttttaggactgggcttcagccttccaagctatgctagtgcg

SEQ ID NO: 69 - DNA, 75 bp, synthetic construct
  source 1..75: mol_type = other DNA
atgaagcgtttcgtctgctttttgttttctcttgtccttggcctgggcttctcacttccatcatatgctagtgcg

SEQ ID NO: 70 - DNA, 75 bp, synthetic construct
  source 1..75: mol_type = other DNA
atgaagaggttcgtatgctttttgttttcacttgtcctgggcctgggcttcagcctcccaagctatgctagtgcg

SEQ ID NO: 71 - DNA, 75 bp, synthetic construct
  source 1..75: mol_type = other DNA
atgaagaggttcgtatgctttttgttttcactcgtcttgggcctgggcttcagcctcccatcatatgctagtgcg

SEQ ID NO: 72 - DNA, 75 bp, synthetic construct
  source 1..75: mol_type = other DNA
atgaagcgtttcgtatgctttttgttttcacttgtccttggcctgggcttcagccttccatcttatgctagtgcg

SEQ ID NO: 73 - DNA, 75 bp, synthetic construct
  source 1..75: mol_type = other DNA
atgaagcgtttcgtctgctttttgttttcacttgtccttggactgggcttctcacttccatcctatgctagtgcg

SEQ ID NO: 74 - DNA, 75 bp, synthetic construct
  source 1..75: mol_type = other DNA
atgaagcgtttcgtatgctttttgttttcacttgtccttggcctgggcttctcacttccatcatatgctagtgcg

SEQ ID NO: 75 - DNA, 78 bp, synthetic construct
  source 1..78: mol_type = other DNA
atgaaagtgaaaagttcgttgatagtgcctatattagcagcaagtatagtagggccagcaatgggaagtgcaattgcg

SEQ ID NO: 76 - DNA, 78 bp, synthetic construct
  source 1..78: mol_type = other DNA
atgaaagtgaaatcaagcttgatcgtgcctatcctggcggctagtattgtaggcccggcgatgggatcagcaattgcg

SEQ ID NO: 77 - DNA, 78 bp, synthetic construct
  source 1..78: mol_type = other DNA
atgaaagtgaaaagtagcttgattgtgcctattcttgcagctagtattgtagggccagcaatgggatcagcaattgcg

SEQ ID NO: 78 - DNA, 78 bp, synthetic construct
  source 1..78: mol_type = other DNA
atgaaagtgaaaagtagcttgattgtgcctatacttgctgctagtattgtaggcccagcgatgggatcagcaattgcg

SEQ ID NO: 79 - DNA, 78 bp, synthetic construct
  source 1..78: mol_type = other DNA
atgaaggtgaaaagtagcttgatagtgcctatactggcggcaagtattgtggggcctgcaatgggatcggcaattgcg

SEQ ID NO: 80 - DNA, 78 bp, synthetic construct
  source 1..78: mol_type = other DNA
atgaaagtgaaaagttcgttgattgtgcctattcttgctgctagtattgtagggccagcgatgggatcagcaattgcg

SEQ ID NO: 81 - DNA, 78 bp, synthetic construct
  source 1..78: mol_type = other DNA
atgaaagtgaaaagtagcttaattgtgcctattcttgcggctagtattgtagggccagcaatgggatcagcaattgcg

SEQ ID NO: 82 - DNA, 78 bp, synthetic construct
  source 1..78: mol_type = other DNA
atgaaagtgaaaagttcgttaatagtgcctatacttgcagctagtattgtagggccagcaatgggaagtgcaattgcg

SEQ ID NO: 83 - DNA, 78 bp, synthetic construct
  source 1..78: mol_type = other DNA
atgaaagtgaaatcaagcttgatagtgcctatacttgcggctagtattgtagggccagcaatgggatcagcaattgcg

SEQ ID NO: 84 - DNA, 78 bp, synthetic construct
  source 1..78: mol_type = other DNA
atgaaagtgaaaagtagcttgatagtccctattctggcggctagtattgtggggccagcgatgggatcggcaattgcg

SEQ ID NO: 85 - DNA, 75 bp, synthetic construct
  source 1..75: mol_type = other DNA
atgaaacgtgtgttgtttagcgtaattgtatttacagccgttggattcacattttgtcaaagtaaagcgcatgcg

SEQ ID NO: 86 - DNA, 75 bp, synthetic construct
  source 1..75: mol_type = other DNA
atgaaacgtgtgttgttttcagttattgtatttacagccgttggattcacattctgtcaaagtaaagcgcatgcg

SEQ ID NO: 87 - DNA, 75 bp, synthetic construct
  source 1..75: mol_type = other DNA
atgaaacgtgtgttgtttagcgttatcgtcttcacagccgttggattcacattctgtcagagtaaagcgcatgcg

SEQ ID NO: 88 - DNA, 75 bp, synthetic construct
  source 1..75: mol_type = other DNA
atgaaacgtgtgttgttcagtgtcatcgtcttcacagccgttggattcacattctgtcagagtaaagcgcatgcg

SEQ ID NO: 89 - DNA, 75 bp, synthetic construct
  source 1..75: mol_type = other DNA
atgaaacgtgtactgttcagcgttatcgtcttcacagccgttggattcacattctgtcagagtaaagcgcatgcg

SEQ ID NO: 90 - DNA, 75 bp, synthetic construct
  source 1..75: mol_type = other DNA
atgaaacgtgtgttgttttcagttattgtcttcacagccgttggattcacattctgtcaaagtaaagcgcatgcg

SEQ ID NO: 91 - DNA, 75 bp, synthetic construct
  source 1..75: mol_type = other DNA
atgaaacgtgtgttgttctctgttattgtattcacggccgttggattcacattctgtcaaagtaaagctcatgcg

SEQ ID NO: 92 - DNA, 75 bp, synthetic construct
  source 1..75: mol_type = other DNA
atgaaacgtgtgttgtttagtgtaattgtatttacagctgtaggattcacattttgtcagagtaaagcgcatgcg

SEQ ID NO: 93 - DNA, 75 bp, synthetic construct
  source 1..75: mol_type = other DNA
atgaaacgtgtgttgttcagtgtaattgtattcacagctgttggattcacattctgtcaaagtaaagcgcatgcg

SEQ ID NO: 94 - DNA, 75 bp, synthetic construct
  source 1..75: mol_type = other DNA
atgaaaagggtgttgtttagtgtaatcgtcttcacagccgttggattcacattctgtcagagtaaagcgcatgcg

SEQ ID NO: 95 - DNA, 75 bp, synthetic construct
  source 1..75: mol_type = other DNA
atgaaacgtgtgttgtttagcgttatcgtattcacagccgttggattcacattctgtcaaagtaaagcgcatgcg

SEQ ID NO: 96 - DNA, 75 bp, synthetic construct
  source 1..75: mol_type = other DNA
atgaaacgtgtgttgtttagcgtaatcgtattcacagccgttggattcacattctgtcaaagtaaagcgcatgcg

SEQ ID NO: 97 - DNA, 75 bp, synthetic construct
  source 1..75: mol_type = other DNA
atgaaacgtgtgttgtttagcgtaatcgtctttacagccgttggattcacattctgtcaaagtaaagcgcatgcg

SEQ ID NO: 98 - DNA, 75 bp, synthetic construct
  source 1..75: mol_type = other DNA
atgaaacgtgtgttgttcagtgttattgttttcacagccgttggattcacattctgtcaaagtaaagcgcatgcg

SEQ ID NO: 99 - DNA, 75 bp, synthetic construct
  source 1..75: mol_type = other DNA
atgtataagaaaattgctattagcatgaccatggccgcgcttctctgcggaatttctatcttccctgcctcagcg

SEQ ID NO: 100 - DNA, 75 bp, synthetic construct
  source 1..75: mol_type = other DNA
atgtataagaaaattgctatcagcatgaccatggccgcgcttttatgcggaatttcaatattccctgcatcagcg

SEQ ID NO: 101 - DNA, 75 bp, synthetic construct
  source 1..75: mol_type = other DNA
atgtacaagaaaattgctatcagcatgacgatggccgctcttttatgcggaatttcaatattcccggcatcagcg

SEQ ID NO: 102 - DNA, 93 bp, synthetic construct
  source 1..93: mol_type = other DNA
atgatggtcattatattgctgattatatctataatcgtcttcctgtatatcatccaacctcagccatctaaaaaccgatctcgccaacaagcg

SEQ ID NO: 103 - DNA, 93 bp, synthetic construct
  source 1..93: mol_type = other DNA
atgatggtaattatcctgctgattatttctattatcgtctttctctatatcatccaacctcagccatctaagaaccgaagccgccaacaagcg

SEQ ID NO: 104 - DNA, 93 bp, synthetic construct
  source 1..93: mol_type = other DNA
atgatggttattatcttgttaattatttcaattattgtctttttatatattatccaacctcaaccatcaaaaaatcgttcaaggcaacaagcg

SEQ ID NO: 105 - DNA, 69 bp, synthetic construct
  source 1..69: mol_type = other DNA
atgaacaaaatttgtcttacgctatgcacggtaagtttatcatttataatttcagcgaacgtccatgcg

SEQ ID NO: 106 - DNA, 69 bp, synthetic construct
  source 1..69: mol_type = other DNA
atgaacaaaatttgtttaacactgtgtacagtaagtctgagtttcataataagtgctaacgtccatgcg

SEQ ID NO: 107 - DNA, 69 bp, synthetic construct
  source 1..69: mol_type = other DNA
atgaacaaaatttgtcttacactctgcactgtatctctgtcatttattattagtgcgaacgtccatgcg

SEQ ID NO: 108 - DNA, 69 bp, synthetic construct
  source 1..69: mol_type = other DNA
atgaacaaaatttgtcttacgctctgcacggtaagtctcagctttataattagcgcgaacgtccatgcg

SEQ ID NO: 109 - DNA, 102 bp, synthetic construct
  source 1..102: mol_type = other DNA
atgaaaaaatggatatttcggattgcggcgttggttataatagttggtgttacgtttttatatgtctctttcaacggcaattttataagcaagaaaatcgcg

SEQ ID NO: 110 - DNA, 102 bp, synthetic construct
  source 1..102: mol_type = other DNA
atgaaaaaatggatatttagaattgcagcattggttataatagttggagttacatttttatatgtctctttcaacgggaattttataagtaaaaaaatcgcg

SEQ ID NO: 111 - DNA, 75 bp, synthetic construct
  source 1..75: mol_type = other DNA
atgaagaaggtgctcattaagatattggtattgacgttattcttgtgtggagtcgtacctgaatttgcgtcagcg

SEQ ID NO: 112 - DNA, 75 bp, synthetic construct
  source 1..75: mol_type = other DNA
atgaaaaaggtgctcattaagatattggtattgacgttattcttgtgtggagtagtgcctgaatttgcttcagcg

SEQ ID NO: 113 - DNA, 75 bp, synthetic construct
  source 1..75: mol_type = other DNA
atgaaaaaagtactaattaagatattggttttgacattattcttatgtggagttgtacctgaatttgctagtgcg

SEQ ID NO: 114 - DNA, 75 bp, synthetic construct
  source 1..75: mol_type = other DNA
atgaaaaaggtactcattaagatattggttctgacattattcttatgtggagtcgtacctgaattcgcttcagcg

SEQ ID NO: 115 - DNA, 75 bp, synthetic construct
  source 1..75: mol_type = other DNA
atgaagaaggtgttaattaagatattggtattgacattattcttatgtggagtagtgcctgaatttgcttcagcg

SEQ ID NO: 116 - DNA, 75 bp, synthetic construct
  source 1..75: mol_type = other DNA
atgaagaaaataaagtatgtagtgttaacagcgattattttctgttgttcaatgtggagttcgagcgttcaagcg

SEQ ID NO: 117 - DNA, 75 bp, synthetic construct
  source 1..75: mol_type = other DNA
atgaagaaaataaagtatgttgtgctgacggccattattttctgctgttcaatgtggagttcgagcgtccaagcg

SEQ ID NO: 118 - DNA, 75 bp, synthetic construct
  source 1..75: mol_type = other DNA
atgaagaaaataaagtatgtagtgttaacagctattattttctgttgttcaatgtggagttcgagtgttcaagcg

SEQ ID NO: 119 - DNA, 75 bp, synthetic construct
  source 1..75: mol_type = other DNA
atgaagaaaataaagtatgtagtgttaactgctattattttctgttgttcaatgtggagttcgagcgttcaagcg

SEQ ID NO: 120 - DNA, 75 bp, synthetic construct
  source 1..75: mol_type = other DNA
atgaagaaaataaagtatgtagtgttaacagctattattttctgttgttcaatgtggagttcgagcgttcaagcg

SEQ ID NO: 121 - DNA, 75 bp, synthetic construct
  source 1..75: mol_type = other DNA
atgaagaaaataaagtatgtagtgttaacagctattattttttgttgttcaatgtggagttcgagcgttcaagcg

SEQ ID NO: 122 - DNA, 75 bp, synthetic construct
  source 1..75: mol_type = other DNA
atgaagaaaataaagtatgtcgtgctgacagccattattttctgttgctcaatgtggagttcgagcgtccaagcg

SEQ ID NO: 123 - DNA, 75 bp, synthetic construct
  source 1..75: mol_type = other DNA
atgaagaaaataaagtatgtagtgttaactgctattattttctgttgttcaatgtggagttcgagtgtgcaagcg

SEQ ID NO: 124 - DNA, 75 bp, synthetic construct
  source 1..75: mol_type = other DNA
atgaagaaaataaagtatgtagtgctgaccgcgattattttctgttgttcaatgtggagttcgagcgttcaagcg

SEQ ID NO: 125 - DNA, 75 bp, synthetic construct
  source 1..75: mol_type = other DNA
atgaagaaaataaagtatgttgtgctgaccgccattattttctgctgctccatgtggagctcgagcgtccaagcg

SEQ ID NO: 126 - DNA, 75 bp, synthetic construct
  source 1..75: mol_type = other DNA
atgaagaaaataaagtatgttgtgttaactgccattattttttgttgttcaatgtggagttcgagcgttcaagcg

SEQ ID NO: 127 - DNA, 75 bp, synthetic construct
  source 1..75: mol_type = other DNA
atgaagaaaataaagtatgttgtgctgaccgctattattttctgttgttctatgtggagttcgagcgttcaagcg

SEQ ID NO: 128 - DNA, 75 bp, synthetic construct
  source 1..75: mol_type = other DNA
atgaaaaaaataaaatatgttgtgttaactgctattattttttgttgttcaatgtggagtagtagtgttcaagcg

SEQ ID NO: 129 - DNA, 75 bp, synthetic construct
  source 1..75: mol_type = other DNA
atgaagaaaataaagtatgtagtgctgaccgccattattttctgctgttccatgtggtcgtcgagcgtccaagcg

SEQ ID NO: 130 - DNA, 90 bp, synthetic construct
  source 1..90: mol_type = other DNA
atgaagggctgtcaaaaaatgatcaaaggcatatttacacgtatcggaatgatgatccttttgttatcattgcctggctatgcttctgcg

SEQ ID NO: 131 - DNA, 90 bp, synthetic construct
  source 1..90: mol_type = other DNA
atgaagggctgtcaaaaaatgatcaaaggtatatttacacgtatcggaatgatgatccttttgttatcattgcctggctatgcttctgcg

SEQ ID NO: 132 - DNA, 90 bp, synthetic construct
  source 1..90: mol_type = other DNA
atgaagggctgtcagaaaatgatcaaaggcatatttacccggatcggcatgatgatcctgctgctgtcactgcccggctatgcgtctgcg

SEQ ID NO: 133 - DNA, 90 bp, synthetic construct
  source 1..90: mol_type = other DNA
atgaagggctgtcaaaaaatgatcaaaggtatatttacacgtattggaatgatgattcttttgttatcacttccgggctatgcttcagcg

SEQ ID NO: 134 - DNA, 90 bp, synthetic construct
  source 1..90: mol_type = other DNA
atgaagggctgtcagaaaatgatcaaaggcatatttacccgtatcggaatgatgatccttctgctgtcacttcctggctatgcgtcagcg

SEQ ID NO: 135 - DNA, 90 bp, synthetic construct
  source 1..90: mol_type = other DNA
atgaagggctgtcaaaaaatgatcaaaggtatatttacacgtatcggaatgatgatccttttgttatcacttccgggctatgcttcagcg

SEQ ID NO: 136 - DNA, 90 bp, synthetic construct
  source 1..90: mol_type = other DNA
atgaaaggctgtcaaaaaatgatcaaaggtatatttacacgtattggaatgatgatccttctgctgtcacttcctggctatgctagtgcg

SEQ ID NO: 137 - DNA, 90 bp, synthetic construct
  source 1..90: mol_type = other DNA
atgaagggctgtcaaaaaatgatcaaaggtatatttaccaggatcggaatgatgatcttactgttatcacttccgggctatgcttcagcg

SEQ ID NO: 138 - DNA, 69 bp, synthetic construct
  source 1..69: mol_type = other DNA
atgaaccgaattttacttacgttaacaacactaatgttcacattattattaagtatcaacgcatttgcg

SEQ ID NO: 139 - DNA, 69 bp, synthetic construct
  source 1..69: mol_type = other DNA
atgaaccgaattttacttacattaacaacactaatgttcacattattattaagtattaacgcatttgcg

SEQ ID NO: 140 - DNA, 69 bp, synthetic construct
  source 1..69: mol_type = other DNA
atgaaccgaattctgcttacactaacaacactaatgttcacattattattaagtattaacgcatttgcg

SEQ ID NO: 141 - DNA, 69 bp, synthetic construct
  source 1..69: mol_type = other DNA
atgaaccgaatactgcttaccttaacaacactaatgttcacattattattaagtattaacgcatttgcg

SEQ ID NO: 142 - DNA, 69 bp, synthetic construct
  source 1..69: mol_type = other DNA
atgaaccgaatactgttaaccctcacaactctaatgttcacattattattaagtatcaacgcatttgcg

SEQ ID NO: 143 - DNA, 69 bp, synthetic construct
  source 1..69: mol_type = other DNA
atgaaccgaatactgcttacactaacaacactaatgttcacattattattaagtattaacgcatttgcg

SEQ ID NO: 144 - DNA, 69 bp, synthetic construct
  source 1..69: mol_type = other DNA
atgaaccgaattttacttacactaacaactctaatgttcactttattattaagtattaacgcatttgcg

SEQ ID NO: 145 - DNA, 69 bp, synthetic construct
  source 1..69: mol_type = other DNA
atgaaccgaatattattaacgctcacaacactaatgttcacattattattaagtatcaacgcatttgcg

SEQ ID NO: 146 - DNA, 69 bp, synthetic construct
  source 1..69: mol_type = other DNA
atgaaccgaattttattaacattaacaacactaatgttcacattattattaagtattaacgcatttgcg

SEQ ID NO: 147 - DNA, 69 bp, synthetic construct
  source 1..69: mol_type = other DNA
atgaacaggattttattaacattaacaacattaatgttcacattattattaagtattaacgcatttgcg

SEQ ID NO: 148 - DNA, 75 bp, synthetic construct
  source 1..75: mol_type = other DNA
atgaaaataatcaaacttgcagcattggcagctcttgtaagtgctcttattttttcgggtattcttgtgttggcg

SEQ ID NO: 149 - DNA, 75 bp, synthetic construct
  source 1..75: mol_type = other DNA
atgaaaataatcaaacttgctgcgttggcagctcttgtgtcagctcttattttctcgggtatccttgtgttggcg

SEQ ID NO: 150 - DNA, 75 bp, synthetic construct
  source 1..75: mol_type = other DNA
atgaaaataatcaaacttgcggcgttggcagcgttggtgtcagcgttgattttttcgggtatccttgtgttggcg

SEQ ID NO: 151 - DNA, 75 bp, synthetic construct
  source 1..75: mol_type = other DNA
atgaaaataatcaaacttgcagcattggcagcacttgtatcagcgttgattttttcgggtattcttgtcttagcg

SEQ ID NO: 152 - DNA, 75 bp, synthetic construct
  source 1..75: mol_type = other DNA
atgaaaataatcaaacttgccgcgttggccgcccttgtatcagcccttattttttcgggcatccttgtgttggcg

SEQ ID NO: 153 - DNA, 75 bp, synthetic construct
  source 1..75: mol_type = other DNA
atgaaaataatcaaacttgccgcgttggcagctcttgtgtcagcccttattttttcgggtatccttgtgttggcg

SEQ ID NO: 154 - DNA, 75 bp, synthetic construct
  source 1..75: mol_type = other DNA
atgaaaataatcaaacttgcggcgttggccgcgttggtgtcagcgttgattttttcgggtatccttgtgttggcg

SEQ ID NO: 155 - DNA, 75 bp, synthetic construct
  source 1..75: mol_type = other DNA
atgaaaataatcaaacttgccgcgttggccgctctggtgtcagccttgattttctcgggtatccttgtgttggcg

SEQ ID NO: 156 - DNA, 75 bp, synthetic construct
  source 1..75: mol_type = other DNA
atgaaaataatcaaacttgccgcattggcagcattggtgtcagcccttatcttttcgggtatccttgtgttggcg

SEQ ID NO: 157 - DNA, 75 bp, synthetic construct
  source 1..75: mol_type = other DNA
atgaaaataatcaaacttgccgcgctggccgccctggtgtcagccttgatcttctcgggtatccttgtgttggcg

SEQ ID NO: 158 - DNA, 75 bp, synthetic construct
  source 1..75: mol_type = other DNA
atgaaaataatcaaacttgccgcgctggccgccttggtgtcagccttgatcttctcgggtatccttgtgttggcg

SEQ ID NO: 159 - DNA, 84 bp, synthetic construct
  source 1..84: mol_type = other DNA
atgaaaaaaaaatcatggtggataatccttttgagcatcgttatcgccgcgtcagccgcggtggctatctacgcaagtagcgcg

SEQ ID NO: 160 - DNA, 84 bp, synthetic construct
  source 1..84: mol_type = other DNA
atgaaaaaaaagtcatggtggataatccttttgagcatcgtcatcgccgcgtcagccgcggtggctatctacgcgagtagtgcg

SEQ ID NO: 161 - DNA, 84 bp, synthetic construct
  source 1..84: mol_type = other DNA
atgaagaaaaaatcatggtggataatcttattgagcatcgttatcgccgcttcagccgcggtggctatctacgcaagtagtgcg

SEQ ID NO: 162 - DNA, 69 bp, synthetic construct
  source 1..69: mol_type = other DNA
atgaaaaaattactgataacgttaacgatcatcatcgcggcggtcttatatgcgcctagtgctcaggcg

SEQ ID NO: 163 - DNA, 69 bp, synthetic construct
  source 1..69: mol_type = other DNA
atgaaaaaattattaataacgttaacgatcatcatcgcggcggtattatatgcgcctagtgctcaggcg

SEQ ID NO: 164 - DNA, 69 bp, synthetic construct
  source 1..69: mol_type = other DNA
atgaaaaaattactaataacgttaacaatcattattgcggcagtcctttatgcgccaagtgctcaggcg

SEQ ID NO: 165 - DNA, 69 bp, synthetic construct
  source 1..69: mol_type = other DNA
atgaaaaaattactcataacgttaacgatcattatcgcggcggtattatatgcgcctagtgctcaggcg

SEQ ID NO: 166 - DNA, 69 bp, synthetic construct
  source 1..69: mol_type = other DNA
atgaaaaaattactgataacgttaacaatcatcatcgcggcggtattatatgcgcctagtgctcaggcg

SEQ ID NO: 167 - DNA, 69 bp, synthetic construct
  source 1..69: mol_type = other DNA
atgaaaaaactgctgataaccctcacaatcattatcgccgcggtcctgtatgcgcctagtgctcaggcg

SEQ ID NO: 168 - DNA, 75 bp, synthetic construct
  source 1..75: mol_type = other DNA
atgaagaagaaagttctggcggcagcaggaattagcggacttttaatggcaggaggagtactgagtgcgtcggcg

SEQ ID NO: 169 - DNA, 96 bp, synthetic construct
  source 1..96: mol_type = other DNA
atgaggaatttaacaaaatacattgtacagttccgggggattgcattctttctatgcttattaatcggatttgcgagcccagccttaacatcagcg

SEQ ID NO: 170 - DNA, 96 bp, synthetic construct
  source 1..96: mol_type = other DNA
atgagaaatttaacgaaatacattgtacagttccgggggattgccttctttctatgcctacttatcggctttgccagcccggccttaacctccgcg

SEQ ID NO: 171 - DNA, 87 bp, synthetic construct
  source 1..87: mol_type = other DNA
atgagggctatttttgggaaaatcacagcggcgttgctgagcattttgttagtcacgaccgggttatttagcccaacgctgaaagcg

SEQ ID NO: 172 - DNA, 87 bp, synthetic construct
  source 1..87: mol_type = other DNA
atgagggctatttttgggaaaatcacagccgcgttgctgtcaattttattagtcacgaccggattgttctcgccgacgttgaaagcg

SEQ ID NO: 173 - DNA, 75 bp, synthetic construct
  source 1..75: mol_type = other DNA
atgattcgctgtagtatcatgattctcgtctcttttatcgtaatgttctctccgttttctaatgtggtcttcgcg

SEQ ID NO: 174 - DNA, 75 bp, synthetic construct
  source 1..75: mol_type = other DNA
atgattcgttgtagtatcatgattttagtaagttttatcgtaatgttcagtcctttttctaatgtggtctttgcg

SEQ ID NO: 175 - DNA, 75 bp, synthetic construct
  source 1..75: mol_type = other DNA
atgatacgatgtagtatcatgattctggtatctttcatcgtaatgttctctccgttttctaatgtggttttcgcg

SEQ ID NO: 176 - DNA, 75 bp, synthetic construct
  source 1..75: mol_type = other DNA
atgatacgatgtagtatcatgattttggtctcttttatcgtaatgttctctccgttttctaatgtggtcttcgcg

SEQ ID NO: 177 - DNA, 75 bp, synthetic construct
  source 1..75: mol_type = other DNA
atgattcgatgtagtatcatgattctcgtctcttttatcgtaatgttctctccgttttctaatgtggtcttcgcg

SEQ ID NO: 178 - DNA, 75 bp, synthetic construct
  source 1..75: mol_type = other DNA
atgatacgctgtagtatcatgattctcgtttcttttatcgtaatgttctctccgttttctaatgttgttttcgcg

SEQ ID NO: 179 - DNA, 75 bp, synthetic construct
  source 1..75: mol_type = other DNA
atgatacgatgtagtatcatgattttagtaagttttatcgtaatgttcagtccgttttctaatgtagtctttgcg

SEQ ID NO: 180 - DNA, 75 bp, synthetic construct
  source 1..75: mol_type = other DNA
atgatacgttgtagtatcatgattttggttagttttatcgttatgttctctccgttttctaatgttgtcttcgcg

SEQ ID NO: 181 - DNA, 75 bp, synthetic construct
  source 1..75: mol_type = other DNA
atgattcgttgtagtatcatgattctagtctcttttatcgtaatgttctctccgttttctaatgtggtctttgcg

SEQ ID NO: 182 - DNA, 75 bp, synthetic construct
  source 1..75: mol_type = other DNA
atgattcgttgtagtatcatgattcttgtcagttttatcgtaatgttcagtccgttttcaaatgtagtctttgcg

SEQ ID NO: 183 - DNA, 75 bp, synthetic construct
  source 1..75: mol_type = other DNA
atgattcgatgttcgatcatgattctggtctcttttatcgtaatgttctctccgttttctaatgtggtcttcgcg

SEQ ID NO: 184 - DNA, 84 bp, synthetic construct
  source 1..84: mol_type = other DNA
atgattgaagtaataacggatatgactatgttatggttcagtgtagtaatgacagtcttattggtagcggcatttgcacgagcg

SEQ ID NO: 185 - DNA, 84 bp, synthetic construct
  source 1..84: mol_type = other DNA
atgattgaggtcataacggatatgactatgctgtggttctctgtcgtaatgaccgtcctgttggtagccgcgttcgcgcgagcg

SEQ ID NO: 186 - DNA, 84 bp, synthetic construct
  source 1..84: mol_type = other DNA
atgattgaagtaataacggatatgactatgctttggttttcagtagtaatgaccgtcttattggtagccgcgtttgcgcgagcg

SEQ ID NO: 187 - DNA, 84 bp, synthetic construct
  source 1..84: mol_type = other DNA
atgattgaggtaattacggatatgactatgctttggttctctgtcgtcatgaccgtcttattggtcgccgcgtttgcgcgagcg

SEQ ID NO: 188 - DNA, 84 bp, synthetic construct
  source 1..84: mol_type = other DNA
atgattgaggtcattacggatatgactatgctttggtttagtgtcgtaatgacagtcttattggtcgccgcgtttgcgcgagcg

SEQ ID NO: 189 - DNA, 84 bp, synthetic construct
  source 1..84: mol_type = other DNA
atgattgaagtaataacggatatgactatgctttggttttcagtcgtaatgaccgtcttattggtagccgcgtttgcgcgagcg

SEQ ID NO: 190 - DNA, 84 bp, synthetic construct
  source 1..84: mol_type = other DNA
atgattgaagtcataacggatatgactatgctgtggttttctgtcgtcatgaccgtcctgttggtcgccgcgtttgcccgggcg

SEQ ID NO: 191 - DNA, 84 bp, synthetic construct
  source 1..84: mol_type = other DNA
atgattgaggtcattacggacatgactatgctttggttttctgtcgtcatgaccgtcttattggtcgccgcgtttgcgcgagcg

SEQ ID NO: 192 - DNA, 84 bp, synthetic construct
  source 1..84: mol_type = other DNA
atgattgaagtaattactgatatgactatgctttggtttagtgttgtaatgacagtcttattggtagcggcatttgcacgagcg

SEQ ID NO: 193 - DNA, 84 bp, synthetic construct
  source 1..84: mol_type = other DNA
atgattgaagtaataacggatatgactatgctttggttttctgtcgtaatgaccgtcttattggttgccgcctttgcgcgagcg

SEQ ID NO: 194 - DNA, 63 bp, synthetic construct
  source 1..63: mol_type = other DNA
atgagaggattcatcctgttgttgttattttttttcttactgtgccagaaaaatgtaggagcg

SEQ ID NO: 195 - DNA, 63 bp, synthetic construct
  source 1..63: mol_type = other DNA
atgagaggattcatcttgttgttgttatttttcttcttgttgtgccagaaaaatgtaggagcg

SEQ ID NO: 196 - DNA, 75 bp, synthetic construct
  source 1..75: mol_type = other DNA
atgaaaacaaaacagacaattgctgcgtcgacactagcgctagcgatgattgcgggagcgaacagtgcgcatgcg

SEQ ID NO: 197 - DNA, 75 bp, synthetic construct
  source 1..75: mol_type = other DNA
atgaaaacaaaacagacaattgctgcttcgacgctagctcttgctatgattgccggagcgaatagtgcacatgcg

SEQ ID NO: 198 - DNA, 75 bp, synthetic construct
  source 1..75: mol_type = other DNA
atgaaaacaaaacagacaattgctgcgtcgacactagctctagcaatgattgcgggagcgaatagtgcgcatgcg

SEQ ID NO: 199 - DNA, 75 bp, synthetic construct
  source 1..75: mol_type = other DNA
atgaaaacaaaacagacaattgctgcatcgacactagctcttgcaatgattgcgggagcaaacagtgcgcatgcg

SEQ ID NO: 200 - DNA, 75 bp, synthetic construct
  source 1..75: mol_type = other DNA
atgaaaacgaaacagacaattgctgcttctacgctagctctagcgatgattgcgggagcgaattctgcacacgcg

SEQ ID NO: 201 - DNA, 75 bp, synthetic construct
  source 1..75: mol_type = other DNA
atgaaaacaaaacagacaattgctgcatcgacgctagctcttgctatgattgcgggagcgaatagtgcacatgcg

SEQ ID NO: 202 - DNA, 75 bp, synthetic construct
  source 1..75: mol_type = other DNA
atgaagacaaaacagacaattgctgcatcgacactagctctagcaatgattgcgggagcaaacagtgcgcatgcg

SEQ ID NO: 203 - DNA, 75 bp, synthetic construct
  source 1..75: mol_type = other DNA
atgaaaacaaaacagacaattgctgcatctacgctagctcttgcgatgattgcgggagcgaattccgcgcacgcg

SEQ ID NO: 204 - DNA, 75 bp, synthetic construct
  source 1..75: mol_type = other DNA
atgaaaacaaaacagacaattgctgcatcgacgctagctcttgcaatgattgcgggagcgaattccgcgcacgcg

SEQ ID NO: 205 - DNA, 75 bp, synthetic construct
  source 1..75: mol_type = other DNA
atgaaaacaaaacagacaattgctgcatctacgctagctcttgctatgattgccggagcgaattctgcacacgcg

SEQ ID NO: 206 - DNA, 75 bp, synthetic construct
  source 1..75: mol_type = other DNA
atgaaaacaaaacagacaattgctgcatcgacgctagcgctagcgatgattgcgggagcgaacagtgcgcacgcg

SEQ ID NO: 207 - DNA, 75 bp, synthetic construct
  source 1..75: mol_type = other DNA
atgaagacaaaacagacaattgctgcatctacgctagcgctagcaatgattgcgggagcaaactctgcgcacgcg

SEQ ID NO: 208 - DNA, 75 bp, synthetic construct
  source 1..75: mol_type = other DNA
atgaaaacgaaacagacaattgcggcgtctacgctcgccctcgcgatgattgcgggagcgaattccgcccacgcg

SEQ ID NO: 209 - DNA, 75 bp, synthetic construct
  source 1..75: mol_type = other DNA
atgaaaacaaaacagacaattgctgcatcgacgctagctttagctatgattgccggagcgaattctgcacatgcg

SEQ ID NO: 210 - DNA, 75 bp, synthetic construct
  source 1..75: mol_type = other DNA
atgaaaaaaacaatcttgttacttttgttgttatttgcattctttttaatgttcttcgctttgccggttttcgcg

SEQ ID NO: 211 - DNA, 75 bp, synthetic construct
  source 1..75: mol_type = other DNA
atgaaaaaaacaatcttgcttcttttgttattatttgcattctttttaatgtttttcgctttgccggttttcgcg

SEQ ID NO: 212 - DNA, 75 bp, synthetic construct
  source 1..75: mol_type = other DNA
atgaaaaaaacaatcctgcttcttttgttattatttgctttctttttaatgttctttgctttgccagttttcgcg

SEQ ID NO: 213 - DNA, 75 bp, synthetic construct
  source 1..75: mol_type = other DNA
atgaaaaaaacaatcttactgcttctgttattatttgcattctttttaatgttcttcgctttgccggtttttgcg

SEQ ID NO: 214 - DNA, 75 bp, synthetic construct
  source 1..75: mol_type = other DNA
atgaaaaaaacaatcttgttacttttgttgttgtttgcattctttttaatgttcttcgctttgccagttttcgcg

SEQ ID NO: 215 - DNA, 75 bp, synthetic construct
  source 1..75: mol_type = other DNA
atgaaaaaaacaatcttgttacttctattattgtttgctttctttttaatgttctttgctttgccggtttttgcg

SEQ ID NO: 216 - DNA, 75 bp, synthetic construct
  source 1..75: mol_type = other DNA
atgaaaaaaacgatcctgctgcttctgctgttgtttgcattctttttgatgttctttgctttgccggtttttgcg

SEQ ID NO: 217 - DNA, 75 bp, synthetic construct
  source 1..75: mol_type = other DNA
atgaaaaagaaagtcgtgattttgttgatgattattgtcttcttcttaatgttctcgacaactcctgcagatgcg

SEQ ID NO: 218 - DNA, 75 bp, synthetic construct
  source 1..75: mol_type = other DNA
atgaaaaaaaaagtcgtgatattgttgatgataattgtcttcttcttaatgttctcgacgactcctgcagacgcg

SEQ ID NO: 219 - DNA, 75 bp, synthetic construct
  source 1..75: mol_type = other DNA
atgaaaaagaaagtagtgattttgttgatgattattgtcttcttcttaatgttctcgacaactcctgcagatgcg

SEQ ID NO: 220 - DNA, 75 bp, synthetic construct
  source 1..75: mol_type = other DNA
atgaaaaagaaagttgtgattttgttaatgattattgtcttcttcttaatgttctcaacaactcctgcagacgcg

SEQ ID NO: 221 - DNA, 75 bp, synthetic construct
  source 1..75: mol_type = other DNA
atgaaaaagaaagtcgtgattttgctgatgattattgtcttcttcttaatgttctctacgactcctgcagacgcg

SEQ ID NO: 222 - DNA, 75 bp, synthetic construct
  source 1..75: mol_type = other DNA
atgaaaaagatggttgcaatcttgttgctgatttttgtcttcttcttgatgttccccgcaatgcctgcatttgcg

SEQ ID NO: 223 - DNA, 75 bp, synthetic construct
  source 1..75: mol_type = other DNA
atgaaaaaaatggttgcaatccttttgttgatatttgttttctttttaatgttcccggcaatgcctgcatttgcg

SEQ ID NO: 224 - DNA, 75 bp, synthetic construct
  source 1..75: mol_type = other DNA
atgaaaaaaatggttgcaattcttttactgatttttgtcttctttcttatgttccctgcaatgcctgcatttgcg

SEQ ID NO: 225 - DNA, 75 bp, synthetic construct
  source 1..75: mol_type = other DNA
atgaaaaagatggtagcaattttgttgttgatttttgtcttctttttaatgttccctgcaatgcctgcatttgcg

SEQ ID NO: 226 - DNA, 75 bp, synthetic construct
  source 1..75: mol_type = other DNA
atgaaaaaaatggttgcaattcttttgttaatttttgtcttctttttaatgttccctgcaatgcctgcatttgcg

SEQ ID NO: 227 - DNA, 75 bp, synthetic construct
  source 1..75: mol_type = other DNA
atgaaaaagatggtcgcaatcctgttgctgatattcgtcttcttcttgatgttccccgcaatgcctgcatttgcg

SEQ ID NO: 228 - DNA, 75 bp, synthetic construct
  source 1..75: mol_type = other DNA
atgaaaaaaatggtcgcaattcttttgttgatttttgtcttctttttaatgttccctgcaatgcctgcatttgcg

SEQ ID NO: 229 - DNA, 78 bp, synthetic construct
  source 1..78: mol_type = other DNA
atgaaaaagacagtcgctatacttttattgttctttgctttcttcttaatgttttcagctttgcctgtgtttgctgcg

SEQ ID NO: 230 - DNA, 78 bp, synthetic construct
  source 1..78: mol_type = other DNA
atgaaaaagacagtagctatccttttattgttctttgcattcttcttaatgttttcagctttgcctgtgtttgcagcg

SEQ ID NO: 231 - DNA, 78 bp, synthetic construct
  source 1..78: mol_type = other DNA
atgaaaaagacagtcgctatccttttactgttctttgccttcttcttaatgttttcagcgttaccggtgtttgctgcg

SEQ ID NO: 232 - DNA, 78 bp, synthetic construct
  source 1..78: mol_type = other DNA
atgaaaaagacagtcgctattcttttattgttctttgcattcttcttaatgttttcagctttgcctgtgtttgctgcg

SEQ ID NO: 233 - DNA, 78 bp, synthetic construct
  source 1..78: mol_type = other DNA
atgaaaaagacagtcgctatccttttattattttttgccttcttcttaatgttttcagctctaccggtgtttgcagcg

SEQ ID NO: 234 - DNA, 78 bp, synthetic construct
  source 1..78: mol_type = other DNA
atgaaaaagacagtcgctattcttttgctgttctttgctttcttcttaatgttctcagctttgcctgtgtttgctgcg

SEQ ID NO: 235 - DNA, 78 bp, synthetic construct
  source 1..78: mol_type = other DNA
atgaaaaagacggtcgcaatccttttgctgttctttgcattcttcttaatgttctcagctttgccggtgttcgcagcg

SEQ ID NO: 236 - DNA, 75 bp, synthetic construct
  source 1..75: mol_type = other DNA
atgaaaaaaacgatcctattgctactgcttctgtttgcatttttcctgatgtttttcgctttaccagtatttgcg

SEQ ID NO: 237 - DNA, 75 bp, synthetic construct
  source 1..75: mol_type = other DNA
atgaaaaaaatggtagcaatcctactgcttatttttgtgtttttcctgatgtttccagctatgccagcttttgcg

SEQ ID NO: 238 - DNA, 75 bp, synthetic construct
  source 1..75: mol_type = other DNA
atgaaaaaaacggtagtattgctaacgcttgttattgcgtttttcggtatgtttttcgtattaccagcttttgcg

SEQ ID NO: 239 - DNA, 75 bp, synthetic construct
  source 1..75: mol_type = other DNA
atgaaaaagatggttgcactgcttagcatgattttcgcattcttcttaatgttctcggtgacgcccgcatttgcg

SEQ ID NO: 240 - DNA, 75 bp, synthetic construct
  source 1..75: mol_type = other DNA
atgaaaaagaaagtagcattgctttttttgagttttgctttcttcttgatgttctcagcgatgcctgcatttgcg

SEQ ID NO: 241 - DNA, 75 bp, synthetic construct
  source 1..75: mol_type = other DNA
atgaaaaagacagttgctagtttgacgatgatttttgcattctttttaatgttctcagtaatgcctgcggatgcg

SEQ ID NO: 242 - DNA, 75 bp, synthetic construct
  source 1..75: mol_type = other DNA
atgaaaaagaaagtggcaatactgcagatgttctttgccttcttcttaatgttctcagcgccgcctgcatttgcg

SEQ ID NO: 243 - DNA, 75 bp, synthetic construct
  source 1..75: mol_type = other DNA
atgaaaaagatggtagcattacttagcatgatttttgcattttttttaatgttttcagtgacacctgcatttgcg

SEQ ID NO: 244 - DNA, 75 bp, synthetic construct
  source 1..75: mol_type = other DNA
atgaaaaagacagtcgcttcattgacgatgatttttgcattctttttaatgttctcagtaatgcctgccgatgcg

SEQ ID NO: 245 - DNA, 75 bp, synthetic construct
  source 1..75: mol_type = other DNA
atgaaaaagacagttgcttcattgacgatgatttttgcattctttttaatgttctcagtaatgcctgcggatgcg

SEQ ID NO: 246 - DNA, 75 bp, synthetic construct
  source 1..75: mol_type = other DNA
atgaaaaagacagtagctagtttgacgatgatttttgcattctttttaatgttctcagtaatgcctgcagatgcg

SEQ ID NO: 247 - DNA, 87 bp, synthetic construct
  source 1..87: mol_type = other DNA
atgatgaggaaaaagagtttttggcttgggatgctgacggccttcatgctcgtgttcacgatggcattcagcgattccgcttctgct

SEQ ID NO: 248 - protein, 27 aa, synthetic construct
MKKVMTSVLVGAIVFTGAGVTAVPVEA

SEQ ID NO: 249 - protein, 22 aa, synthetic construct
MRFISCVILVVSIMIPALDTKA

SEQ ID NO: 250 - protein, 29 aa, synthetic construct
MRKVVTYSLAAAIGLSGLIGFAGVGSVSA

SEQ ID NO: 251 - protein, 33 aa, synthetic construct
MLKKAKGKVIAGTLVVGLVAGGGAVLGATDAGA

SEQ ID NO: 252 - protein, 25 aa, synthetic construct
MKTKQTIAASTLALAMIAVANSAHA

SEQ ID NO: 253 - protein, 20 aa, synthetic construct
MKKQIAILAVSVAFFIGVWA

SEQ ID NO: 254 - protein, 25 aa, synthetic construct
MKKVQCFILMAVILCSSMWSATVKA

SEQ ID NO: 255 - protein, 27 aa, synthetic construct
MKRTLVMTTTLIITILIVSLFLRFGSA

SEQ ID NO: 256 - protein, 25 aa, synthetic construct
MKRFVCFLFSLVLGLGFSLPSYASA

SEQ ID NO: 257 - protein, 26 aa, synthetic construct
MKVKSSLIVPILAASIVGPAMGSAIA

SEQ ID NO: 258 - protein, 25 aa, synthetic construct
MKRVLFSVIVFTAVGFTFCQSKAHA

SEQ ID NO: 259 - protein, 25 aa, synthetic construct
MYKKIAISMTMAALLCGISIFPASA

SEQ ID NO: 260 - protein, 31 aa, synthetic construct
MMVIILLIISIIVFLYIIQPQPSKNRSRQQA

SEQ ID NO: 261 - protein, 23 aa, synthetic construct
MNKICLTLCTVSLSFIISANVHA

SEQ ID NO: 262 - protein, 34 aa, synthetic construct
MKKWIFRIAALVIIVGVTFLYVSFNGNFISKKIA

SEQ ID NO: 263 - protein, 25 aa, synthetic construct
MKKVLIKILVLTLFLCGVVPEFASA

SEQ ID NO: 264 - protein, 25 aa, synthetic construct
MKKIKYVVLTAIIFCCSMWSSSVQA

SEQ ID NO: 265 - protein, 30 aa, synthetic construct
MKGCQKMIKGIFTRIGMMILLLSLPGYASA

SEQ ID NO: 266 - protein, 23 aa, synthetic construct
MNRILLTLTTLMFTLLLSINAFA

SEQ ID NO: 267 - protein, 25 aa, synthetic construct
MKIIKLAALAALVSALIFSGILVLA

SEQ ID NO: 268 - protein, 28 aa, synthetic construct
MKKKSWWIILLSIVIAASAAVAIYASSA

SEQ ID NO: 269 - protein, 23 aa, synthetic construct
MKKLLITLTIIIAAVLYAPSAQA

SEQ ID NO: 270 - protein, 25 aa, synthetic construct
MKKKVLAAAGISGLLMAGGVLSASA

SEQ ID NO: 271 - protein, 32 aa, synthetic construct
MRNLTKYIVQFRGIAFFLCLLIGFASPALTSA

SEQ ID NO: 272 - protein, 29 aa, synthetic construct
MRAIFGKITAALLSILLVTTGLFSPTLKA

SEQ ID NO: 273 - protein, 25 aa, synthetic construct
MIRCSIMILVSFIVMFSPFSNVVFA

SEQ ID NO: 274 - protein, 28 aa, synthetic construct
MIEVITDMTMLWFSVVMTVLLVAAFARA

SEQ ID NO: 275 - protein, 21 aa, synthetic construct
MRGFILLLLFFFLLCQKNVGA

SEQ ID NO: 276 - protein, 25 aa, synthetic construct
MKTKQTIAASTLALAMIAGANSAHA

SEQ ID NO: 277 - protein, 25 aa, synthetic construct
MKKTILLLLLLFAFFLMFFALPVFA

SEQ ID NO: 278 - protein, 25 aa, synthetic construct
MKKKVVILLMIIVFFLMFSTTPADA

SEQ ID NO: 279 - protein, 25 aa, synthetic construct
MKKMVAILLLIFVFFLMFPAMPAFA

SEQ ID NO: 280 - protein, 26 aa, synthetic construct
MKKTVAILLLFFAFFLMFSALPVFAA

SEQ ID NO: 281 - protein, 25 aa, synthetic construct
MKKTILLLLLLFAFFLMFFALPVFA

SEQ ID NO: 282 - protein, 25 aa, synthetic construct
MKKMVAILLLIFVFFLMFPAMPAFA

SEQ ID NO: 283 - protein, 25 aa, synthetic construct
MKKTVVLLTLVIAFFGMFFVLPAFA

SEQ ID NO: 284 - protein, 25 aa, synthetic construct
MKKMVALLSMIFAFFLMFSVTPAFA

SEQ ID NO: 285 - protein, 25 aa, synthetic construct
MKKKVALLFLSFAFFLMFSAMPAFA

SEQ ID NO: 286 - protein, 25 aa, synthetic construct
MKKTVASLTMIFAFFLMFSVMPADA

SEQ ID NO: 287 - protein, 25 aa, synthetic construct
MKKKVAILQMFFAFFLMFSAPPAFA

SEQ ID NO: 288 - protein, 25 aa, synthetic construct
MKKMVALLSMIFAFFLMFSVTPAFA

SEQ ID NO: 289 - protein, 25 aa, synthetic construct
MKKTVASLTMIFAFFLMFSVMPADA

SEQ ID NO: 290 - protein, 29 aa, synthetic construct
MMRKKSFWLGMLTAFMLVFTMAFSDSASA

SEQ ID NO: 291 - DNA, 1143 bp, synthetic construct
  source 1..1143: mol_type = other DNA
atgaaaaaaccgctggggaaaattgtcgcaagcaccgcactactcatttctgttgcttttagttcatcgatcgcatcggctgctgaagaagcaaaagaaaaatatttaattggctttaatgagcaggaagctgtcagtgagtttgtagaacaagtagaggcaaatgacgaggtcgccattctctctgaggaagaggaagtcgaaattgaattgcttcatgaatttgaaacgattcctgttttatccgttgagttaagcccagaagatgtggacgcgcttgaactcgatccagcgatttcttatattgaagaggatgcagaagtaacgacaatggcgcaatcggtaccatggggaattcgccgtgtgcaagccccaactgcccataaccgtggattgacaggttctggtgtaaaagttgctgtcctcgatacagggatatccactcatccagatctaaatattcgtggtggcgcaagctttgtaccaggggaaccgtcgactcaagatgaaaatgggcatggcacgcatgcggccgggacgatcgctgctttaaacaattcgattggcgttcttggcgtagctcctagcgctgagctatacgctgttaaagtcctaggagcaagcggttcaggttcggtcagctcgattgcccaaggattggaatgggcagggaacaatggcatgcacgttgctaatttgagtttaggaagcccttcgccaagtgccacactcgagcaagctgttaatagcgcgacttctagaggcgttcttgttgtagcggcatctgggaattcaggtgcaggctcaatcagctatccggcgcgctatgcgaacgcaatggcagtcggagctactgatcaaaacaacaaccgcgctagcttttcacagtatggcccaggccttgacattgtcgcacccggggtaaacattcagagcacatacccaggttcaacatatgccagcttgaacggtacatcgatggctactcctcatgttgcaggtgcggccgcccttgttaaacaaaagaacccatcttggtctaatgtacgtattcgaaatcatctaaagaatacggcaactagtttaggaagcacggacttgtatggaagcggacttgttaacgcagaagcggcaacgcgataa

SEQ ID NO: 292 - protein, 380 aa, synthetic construct
MKKPLGKIVASTALLISVAFSSSIASAAEEAKEKYLIGFNEQEAVSEFVEQVEANDEVAILSEEEEVEIELLHEFETIPVLSVELSPEDVDALELDPAISYIEEDAEVTTMAQSVPWGIRRVQAPTAHNRGLTGSGVKVAVLDTGISTHPDLNIRGGASFVPGEPSTQDENGHGTHAAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGPGLDIVAPGVNIQSTYPGSTYASLNGTSMATPHVAGAAALVKQKNPSWSNVRIRNHLKNTATSLGSTDLYGSGLVNAEAATR